(19) United States
(12) Patent Application Publication
(10) Pub. No.: US 2026/0176660 A1
(43) Pub. Date: Jun. 25, 2026

(54) MICROBIAL PRODUCTION OF CANNABINOIDS

(71) Applicant: Amyris, Inc., Emeryville, CA (US)

(72) Inventors: Victor F. HOLMES, Emeryville, CA (US); John E. HUNG, Emeryville, CA (US); Irina A. KORYAKINA, Emeryville, CA (US); Andrew P. KLEIN, Emeryville, CA (US); Yi XIONG, Emeryville, CA (US); William E. DRAPER, Emeryville, CA (US); Hailley S. WARBINGTON, Emeryville, CA (US)

(21) Appl. No.: 19/416,289

(22) Filed: Dec. 11, 2025

Related U.S. Application Data
(63) Continuation of application No. 18/022,063, now Pat. No. 12,522,849.
(60) Provisional application No. 63/067,633, filed on Aug. 19, 2020.

(51) Int. Cl. C12P 7/42; C12N 1/185; C12N 9/00; C12N 9/02; C12N 9/10; C12N 9/88

(52) U.S. Cl. CPC C12P 7/42; C12N 1/185; C12N 9/0004; C12N 9/1029; C12N 9/88; C12N 9/93; C12Y 121/03008; C12Y 404/01026

(57) ABSTRACT

The compositions and methods of the disclosure can be used to produce a cannabinoid in a host cell, such as a yeast cell. For example, the disclosure features host cells (e.g., yeast cells) modified to express one or more enzymes of a cannabinoid biosynthetic pathway, such as an acyl activating enzyme (AAE), a tetraketide synthase (TKS), a cannabigerolic acid synthase (CBGaS), and/or an olivetolic acid cyclase (OAC).

### SEQUENCE LISTING

The instant application contains a Sequence Listing which has been submitted electronically in XML format and is hereby incorporated by reference in its entirety. Said XML copy, created on Jan. 7, 2026, is named 51494-008003_Sequence_Listing_1_7_26 and is 100,624 bytes in size.

### BACKGROUND OF THE INVENTION

Cannabinoids are chemical compounds such as cannabigerols (CBG), cannabichromens (CBC), cannabidiol (CBD), tetrahydrocannabinol (THC), cannabinol (CBN), cannabinodiol (CBDL), cannabicyclol (CBL), cannabielsoin (CBE), cannabitriol (CBT), and tetrahydrocannabinolic acid (THCa), which are produced by the cannabis plant. Cannabinoids may be used to improve various aspects of human health. However, producing cannabinoids in preparative amounts and in high yield has been challenging. There remains a need for compositions and methods capable of preparing cannabinoids with high efficiency and chemical selectivity.

### SUMMARY OF THE INVENTION

The present disclosure provides compositions and methods for producing a cannabinoid in a host cell, such as a yeast cell. For example, using the compositions and methods described herein, a yeast cell may be modified to express one or more enzymes of a cannabinoid biosynthetic pathway, such as an acyl activating enzyme (AAE), a tetraketide synthase (TKS), a cannabigerolic acid synthase (CBGaS), and/or an olivetolic acid cyclase (OAC), among others described herein. The yeast cell may then be cultured, for example, in the presence of an agent that regulates expression of the one or more enzymes. The yeast cell may be incubated for a time sufficient to allow for biochemical synthesis of a cannabinoid, and the cannabinoid may then be separated from the yeast cell.

In one aspect, the disclosure features a host cell capable of producing a cannabinoid. The host cell may contain one or more heterologous nucleic acids that each, independently, encode an acyl activating enzyme (AAE) having an amino acid sequence that is at least 85% identical to the amino acid sequence of any one of SEQ ID NOS: 1-24. In some embodiments, the AAE has an amino acid sequence that is at least 85% identical to the amino acid sequence of any one of any one of SEQ ID NOS: 1-5 and 7-24 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 1-5 and 7-24). For example, the AAE may have an amino acid sequence that is at least 85% identical to the amino acid sequence of any one of SEQ ID NO: 1-4.

Additionally or alternatively, the host cell may contain one or more heterologous nucleic acids that each, independently, encode a tetraketide synthase (TKS) having an amino acid sequence that is at least 85% identical to the amino acid sequence of any one of SEQ ID NOS: 25-43. In some embodiments, the TKS has an amino acid sequence that is at least 85% identical to the amino acid sequence of any one of SEQ ID NOS: 25 and 27-43 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 25 and 27-43).

Additionally or alternatively, the host cell may contain one or more heterologous nucleic acids that each, independently, encode a cannabigerolic acid synthase (CBGaS) having an amino acid sequence that is at least 85% identical to the amino acid sequence of any one of SEQ ID NOS: 53-58, 63, and 64. In some embodiments, the CBGaS has an amino acid sequence that is at least 85% identical to the amino acid sequence of any one of SEQ ID NOS: 55-58, 63, and 64 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 55-58, 63, and 64).

Additionally or alternatively, the host cell may contain one or more heterologous nucleic acids that each, independently, encode an olivetolic acid cyclase (OAC) having an amino acid sequence that is at least 85% identical to the amino acid sequence of any one of SEQ ID NOS: 44-52. In some embodiments, the OAC has an amino acid sequence that is at least 85% identical to the amino acid sequence of any one of SEQ ID NOS: 45-52 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 45-52).

In some embodiments, the host cell contains a heterologous nucleic acid that encodes an AAE having an amino acid sequence that is at least 90% identical to the amino acid sequence of any one of SEQ ID NOS: 1-5 and 7-24 (e.g., an amino acid sequence that is 90%, 91%, 92%, 93%, 94%, 95%, 96%, 97%, 98%, 99%, or 100% identical to any one of SEQ ID NOS: 1-5 and 7-24). For example, the AAE may have an amino acid sequence that is at least 90% identical to the amino acid sequence of any one of SEQ ID NO: 1-4. In some embodiments, the AAE has an amino acid sequence that is at least 95% identical to the amino acid sequence of any one of SEQ ID NO: 1-4 (e.g., an amino acid sequence that is 95%, 96%, 97%, 98%, 99%, or 100% identical to any one of SEQ ID NO: 1-4). In some embodiments, the AAE has the amino acid sequence of any one of SEQ ID NO: 1-4.

In some embodiments, the host cell contains a heterologous nucleic acid that encodes a TKS having an amino acid sequence that is at least 90% identical to the amino acid sequence of any one of SEQ ID NOS: 25 and 34-39 (e.g., an amino acid sequence that is 90%, 91%, 92%, 93%, 94%, 95%, 96%, 97%, 98%, 99%, or 100% identical to any one of SEQ ID NOS: 25 and 34-39). In some embodiments, the TKS has an amino acid sequence that is at least 95% identical to the amino acid sequence of any one of SEQ ID NOS: 25 and 34-39 (e.g., an amino acid sequence that is 95%, 96%, 97%, 98%, 99%, or 100% identical to any one of SEQ ID NOS: 25 and 34-39). In some embodiments, the TKS has the amino acid sequence of any one of SEQ ID NOS: 25 and 34-39.

In some embodiments, the host cell contains a heterologous nucleic acid that encodes a TKS having an amino acid sequence that is at least 90% identical to the amino acid sequence of any one of SEQ ID NO: 25 or 39 (e.g., an amino acid sequence that is 90%, 91%, 92%, 93%, 94%, 95%, 96%, 97%, 98%, 99%, or 100% identical to any one of SEQ ID NO: 25 or 39). In some embodiments, the TKS has an amino acid sequence that is at least 95% identical to the amino acid sequence of any one of SEQ ID NO: 25 or 39 (e.g., an amino acid sequence that is 95%, 96%, 97%, 98%, 99%, or 100% identical to any one of SEQ ID NO: 25 or 39). In some embodiments, the TKS has the amino acid sequence of any one of SEQ ID NO: 25 or 39.

In some embodiments, the host cell contains a heterologous nucleic acid that encodes a CBGaS having an amino acid sequence that is at least 90% identical to the amino acid sequence of any one of SEQ ID NOS: 55-58, 63, and 64 (e.g., an amino acid sequence that is 90%, 91%, 92%, 93%, 94%, 95%, 96%, 97%, 98%, 99%, or 100% identical to any one of SEQ ID NOS: 55-58, 63, and 64). In some embodiments, the CBGaS has an amino acid sequence that is at least 95% identical to the amino acid sequence of any one of SEQ ID NOS: 55-58, 63, and 64 (e.g., an amino acid sequence that is 95%, 96%, 97%, 98%, 99%, or 100% identical to any one of SEQ ID NOS: 55-58, 63, and 64). In some embodiments, the CBGaS has the amino acid sequence of any one of SEQ ID NOS: 55-58, 63, and 64.

In some embodiments, the host cell contains a heterologous nucleic acid that encodes an OAC having an amino acid sequence that is at least 90% identical to the amino acid sequence of any one of SEQ ID NOS: 45-52 (e.g., an amino acid sequence that is 90%, 91%, 92%, 93%, 94%, 95%, 96%, 97%, 98%, 99%, or 100% identical to any one of SEQ ID NOS: 45-52). In some embodiments, the OAC has an amino acid sequence that is at least 95% identical to the amino acid sequence of any one of SEQ ID NOS: 45-52 (e.g., an amino acid sequence that is 95%, 96%, 97%, 98%, 99%, or 100% identical to any one of SEQ ID NOS: 45-52). In some embodiments, OAC has the amino acid sequence of any one of SEQ ID NOS: 45-52.

In some embodiments, the host cell further contains one or more heterologous nucleic acids that each, independently, encode an enzyme of the mevalonate biosynthetic pathway. The enzyme may be, for example, an acetyl-CoA thiolase, an HMG-COA synthase, an HMG-CoA reductase, a mevalonate kinase, a phosphomevalonate kinase, a mevalonate pyrophosphate decarboxylase, or an IPP:DMAPP isomerase. In some embodiments, the host cell contains heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

In some embodiments, the host cell further contains a heterologous nucleic acid that encodes geranyl pyrophosphate (GPP) synthase. In some embodiments, the GPP synthase has an amino acid sequence that is at least 90% identical to the amino acid sequence of SEQ ID NO: 75 (e.g., an amino acid sequence that is 90%, 91%, 92%, 93%, 94%, 95%, 96%, 97%, 98%, 99%, or 100% identical SEQ ID NO: 75). In some embodiments, the GPP synthase has an amino acid sequence that is at least 95% identical to the amino acid sequence of SEQ ID NO: 75 (e.g., an amino acid sequence that is 95%, 96%, 97%, 98%, 99%, or 100% identical to SEQ ID NO: 75). In some embodiments, the GPP synthase has an amino acid sequence of SEQ ID NO: 75.

In some embodiments, the host cell further contains one or more heterologous nucleic acids that each, independently, encode an acetyl-CoA synthase, and/or an aldehyde dehydrogenase, and/or a pyruvate decarboxylase. In some embodiments, the acetyl-CoA synthase has an amino acid sequence that is at least 90% identical to the amino acid sequence of SEQ ID NO: 66 (e.g., an amino acid sequence that is 90%, 91%, 92%, 93%, 94%, 95%, 96%, 97%, 98%, 99%, or 100% identical to SEQ ID NO: 66). In some embodiments, the acetyl-CoA synthase has an amino acid sequence that is at least 95% identical to the amino acid sequence of SEQ ID NO: 66 (e.g., an amino acid sequence that is 95%, 96%, 97%, 98%, 99%, or 100% identical to SEQ ID NO: 66). In some embodiments, the acetyl-CoA synthase has the amino acid sequence of SEQ ID NO: 66. In some embodiments, the aldehyde dehydrogenase has an amino acid sequence that is at least 90% identical to the amino acid sequence of SEQ ID NO: 67 (e.g., an amino acid sequence that is 90%, 91%, 92%, 93%, 94%, 95%, 96%, 97%, 98%, 99%, or 100% identical to SEQ ID NO: 67). In some embodiments, the aldehyde dehydrogenase has an amino acid sequence that is at least 95% identical to the amino acid sequence of SEQ ID NO: 67 (e.g., an amino acid sequence that is 95%, 96%, 97%, 98%, 99%, or 100% identical to SEQ ID NO: 67). In some embodiments, the aldehyde dehydrogenase synthase has the amino acid sequence of SEQ ID NO: 67. In some embodiments, the pyruvate decarboxylase has an amino acid sequence that is at least 90% identical to the amino acid sequence of SEQ ID NO: 65 (e.g., an amino acid sequence that is 90%, 91%, 92%, 93%, 94%, 95%, 96%, 97%, 98%, 99%, or 100% identical to SEQ ID NO: 65). In some embodiments, the pyruvate decarboxylase has an amino acid sequence that is at least 95% identical to the amino acid sequence of SEQ ID NO: 65 (e.g., an amino acid sequence that is 95%, 96%, 97%, 98%, 99%, or 100% identical to SEQ ID NO: 65). In some embodiments, the pyruvate decarboxylase has the amino acid sequence of SEQ ID NO: 65.

In some embodiments, expression of the one or more heterologous nucleic acids is regulated by an exogenous agent. In some embodiments, the exogenous agent decreases production of the cannabinoid. In some embodiments, the exogenous agent increases production of the cannabinoid. In some embodiments, the exogenous agent is galactose and expression of at least one of the one or more heterologous nucleic acids is under the control of a GAL promoter. In some embodiments, expression of at least one of the one or more heterologous nucleic acids is under the control of a galactose-responsive promoter. In some embodiments, expression of at least one of the one or more heterologous nucleic acids is under the control of a maltose-responsive promoter. In some embodiments, expression of at least one of the one or more heterologous nucleic acids is under the control of a combination of both a galactose-responsive promoter and a maltose-responsive promoter.

In some embodiments, the cannabinoid is cannabidiolic acid (CBDA), cannabidiol (CBD), cannabigerolic acid (CBGA), cannabigerol (CBG), tetrahydrocannabinol (THC), tetrahydrocannabinolic acid (THCa), cannabigerorcinic acid (CBGOA), cannabigerovarinic acid (CBGVA), or 3-geranyl-2,4-dihydroxy-6-phenylethylbenzoic acid (CBGXA).

In some embodiments, the cannabinoid is CBGA, CBG, sesquicannabigerolic acid (SCBGA), CBGOA, sesquicannabigerorcinic acid (SCBGOA), CBGVA, sesquicannabigerovarinic acid (SCBGVA), CBGXA, or 3-farnesyl-2,4-dihydroxy-6-phenylethylbenzoic acid (SCBGXA).

In some embodiments, the host cell is a yeast cell, such as a yeast cell belonging to a yeast strain described herein. In some embodiments, the yeast cell is S. cerevisiae.

In another aspect, the disclosure features a mixture containing the host cell of any one of the above aspects or embodiments of the disclosure and a culture medium. In some embodiments, the cell culture medium further contains an exogenous agent, such as maltose. In some embodiments, the exogenous agent is maltose. In some embodiments, the culture medium contains (i) an exogenous agent that increases production of the cannabinoid, and (ii) a precursor required to make the cannabinoid. In some embodiments, the precursor required to make the cannabinoid is hexanoate.

In another aspect, the disclosure features a method for decreasing expression of a cannabinoid in a host cell by culturing the host cell of any of the above aspects or embodiments of the disclosure in a medium comprising an exogenous agent. The exogenous agent may be one, for example, that decreases the expression of the cannabinoid. In some embodiments, the exogenous agent is maltose. In some embodiments, culturing the host cell in the medium comprising the exogenous agent results in production of less than 0.001 mg/L of cannabinoid.

In another aspect, the disclosure features a method for increasing expression of a cannabinoid in a host cell by culturing the host cell of any of the above aspects or embodiments of the disclosure in a medium comprising an exogenous agent. The exogenous agent may be one that, for example, increases the expression of the cannabinoid. In some embodiments, the exogenous agent is galactose. In some embodiments, the method further includes culturing the host cell with a precursor required to make the cannabinoid, such as hexanoate.

In another aspect, the disclosure features a method of genetically modifying a host cell to be capable of producing a cannabinoid. The method may include introducing into the host cell one or more heterologous nucleic acids that each, independently, encode an AAE having an amino acid sequence that is at least 85% identical to the amino acid sequence of any one of SEQ ID NOS: 1-24. In some embodiments, the AAE has an amino acid sequence that is at least 85% identical to the amino acid sequence of any one of SEQ ID NOS: 1-5 and 7-24 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 1-5 and 7-24). For example, the AAE may have an amino acid sequence that is at least 85% identical to the amino acid sequence of any one of SEQ ID NO: 1-4.

Additionally or alternatively, the method may include introducing into the host cell one or more heterologous nucleic acids that each, independently, encode a TKS having an amino acid sequence that is at least 85% identical to the amino acid sequence of any one of SEQ ID NOS: 25-43. In some embodiments, the TKS has an amino acid sequence that is at least 85% identical to the amino acid sequence of any one of SEQ ID NOS: 25 and 27-43 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 25 and 27-43).

Additionally or alternatively, the method may include introducing into the host cell one or more heterologous nucleic acids that each, independently, encode a CBGaS having an amino acid sequence that is at least 85% identical to the amino acid sequence of any one of SEQ ID NOS: 53-58, 63, or 64. In some embodiments, the CBGaS has an amino acid sequence that is at least 85% identical to the amino acid sequence of any one of SEQ ID NOS: SEQ ID NOS: 55-58, 63, and 64 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 55-58, 63, and 64).

Additionally or alternatively, the method may include introducing into the host cell one or more heterologous nucleic acids that each, independently, encode an OAC having an amino acid sequence that is at least 85% identical to the amino acid sequence of any one of SEQ ID NO: 44-52. In some embodiments, the OAC has an amino acid sequence that is at least 85% identical to the amino acid sequence of any one of SEQ ID NOS: 45-52 (e.g., an amino acid sequence that is 85%, 86%, 87%, 88%, 89%, 90%, 91%, 92%, 93%, 94%, 95%, 96%, 97%, 98%, 99%, or 100% identical to any one of SEQ ID NOS: 45-52).

In some embodiments, the method includes introducing into the host cell a heterologous nucleic acid that encodes an AAE having an amino acid sequence that is at least 90% identical to the amino acid sequence of any one of SEQ ID NOS: 1-5 and 7-24 (e.g., an amino acid sequence that is 90%, 91%, 92%, 93%, 94%, 95%, 96%, 97%, 98%, 99%, or 100% identical to any one of SEQ ID NOS: 1-5 and 7-24). For example, the AAE may have an amino acid sequence that is at least 90% identical to the amino acid sequence of any one of SEQ ID NO: 1-4. In some embodiments, the AAE has an amino acid sequence that is at least 95% identical to the amino acid sequence of any one of SEQ ID NO: 1-4 (e.g., an amino acid sequence that is 95%, 96%, 97%, 98%, 99%, or 100% identical to any one of SEQ ID NO: 1-4). In some embodiments, the AAE has the amino acid sequence of any one of SEQ ID NO: 1-4.

In some embodiments, the method includes introducing into the host cell a heterologous nucleic acid that encodes a TKS having an amino acid sequence that is at least 90% identical to the amino acid sequence of any one of SEQ ID NOS: 25 and 27-43 (e.g., an amino acid sequence that is 90%, 91%, 92%, 93%, 94%, 95%, 96%, 97%, 98%, 99%, or 100% identical to any one of SEQ ID NOS: 25 and 27-43). In some embodiments, the TKS has an amino acid sequence that is at least 95% identical to the amino acid sequence of any one of SEQ ID NOS: 25 and 27-43 (e.g., an amino acid sequence that is 95%, 96%, 97%, 98%, 99%, or 100% identical to any one of SEQ ID NOS: 25 and 27-43). In some embodiments, the TKS has the amino acid sequence of any one of SEQ ID NOS: 25 and 27-43.

In some embodiments, the method includes introducing into the host cell a heterologous nucleic acid that encodes a TKS having an amino acid sequence that is at least 90% identical to the amino acid sequence of any one of SEQ ID NOS: 25 or 39 (e.g., an amino acid sequence that is 90%, 91%, 92%, 93%, 94%, 95%, 96%, 97%, 98%, 99%, or 100% identical to any one of SEQ ID NOS: 25 or 39). In some embodiments, the TKS has an amino acid sequence that is at least 95% identical to the amino acid sequence of any one of SEQ ID NOS: 25 or 39 (e.g., an amino acid sequence that is 95%, 96%, 97%, 98%, 99%, or 100% identical to any one of SEQ ID NOS: 25 or 39). In some embodiments, the TKS has the amino acid sequence of any one of SEQ ID NOS: 25 or 39.

In some embodiments, the method includes introducing into the host cell a heterologous nucleic acid that encodes a CBGaS having an amino acid sequence that is at least 90% identical to the amino acid sequence of any one of SEQ ID NOS: 55-58, 63, and 64 (e.g., an amino acid sequence that is 90%, 91%, 92%, 93%, 94%, 95%, 96%, 97%, 98%, 99%, or 100% identical to any one of SEQ ID NOS: 55-58, 63, and 64). In some embodiments, the CBGaS has an amino acid sequence that is at least 95% identical to the amino acid sequence of any one of SEQ ID NOS: 55-58, 63, and 64 (e.g., an amino acid sequence that is 95%, 96%, 97%, 98%, 99%, or 100% identical to any one of SEQ ID NOS: 55-58, 63, and 64). In some embodiments, the CBGaS has the amino acid sequence of any one of SEQ ID NOS: 55-58, 63, and 64.

In some embodiments, the method includes introducing into the host cell a heterologous nucleic acid that encodes an OAC having an amino acid sequence that is at least 90% identical to the amino acid sequence of any one of SEQ ID NOS: 45-52 (e.g., an amino acid sequence that is 90%, 91%, 92%, 93%, 94%, 95%, 96%, 97%, 98%, 99%, or 100% identical to any one of SEQ ID NOS: 45-52). In some embodiments, the OAC has an amino acid sequence that is at least 95% identical to the amino acid sequence of any one of SEQ ID NOS: 45-52 (e.g., an amino acid sequence that is 95%, 96%, 97%, 98%, 99%, or 100% identical to any one of SEQ ID NOS: 45-52). In some embodiments, OAC has the amino acid sequence of any one of SEQ ID NOS: 45-52.

In some embodiments, the host cell further contains one or more heterologous nucleic acids that each, independently, encode an enzyme of the mevalonate biosynthetic pathway, such as an enzyme selected from an acetyl-CoA thiolase, an HMG-COA synthase, an HMG-COA reductase, a mevalonate kinase, a phosphomevalonate kinase, a mevalonate pyrophosphate decarboxylase, and an IPP:DMAPP isomerase. In some embodiments, the host cell contains heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

In some embodiments, the host cell further contains a heterologous nucleic acid that encodes a GPP synthase. In some embodiments, the GPP synthase has an amino acid sequence that is at least 90% identical to the amino acid sequence of SEQ ID NO: 75 (e.g., an amino acid sequence that is 90%, 91%, 92%, 93%, 94%, 95%, 96%, 97%, 98%, 99%, or 100% identical to SEQ ID NO: 75). In some embodiments, the GPP synthase has an amino acid sequence that is at least 95% identical to the amino acid sequence of SEQ ID NO: 75 (e.g., an amino acid sequence that is 95%, 96%, 97%, 98%, 99%, or 100% identical to SEQ ID NO: 75). In some embodiments, the GPP synthase has an amino acid sequence of SEQ ID NO: 75.

In some embodiments, the host cell further contains one or more heterologous nucleic acids that each, independently, encode an acetyl-CoA synthase, and/or an aldehyde dehydrogenase, and/or a pyruvate decarboxylase. In some embodiments, the acetyl-CoA synthase has an amino acid sequence that is at least 90% identical to the amino acid sequence of SEQ ID NO: 66 (e.g., an amino acid sequence that is 90%, 91%, 92%, 93%, 94%, 95%, 96%, 97%, 98%, 99%, or 100% identical to SEQ ID NO: 66). In some embodiments, the acetyl-CoA synthase has an amino acid sequence that is at least 95% identical to the amino acid sequence of SEQ ID NO: 66 (e.g., an amino acid sequence that is 95%, 96%, 97%, 98%, 99%, or 100% identical to SEQ ID NO: 66). In some embodiments, the acetyl-CoA synthase has the amino acid sequence of SEQ ID NO: 66. In some embodiments, the aldehyde dehydrogenase has an amino acid sequence that is at least 90% identical to the amino acid sequence of SEQ ID NO: 67 (e.g., an amino acid sequence that is 90%, 91%, 92%, 93%, 94%, 95%, 96%, 97%, 98%, 99%, or 100% identical to SEQ ID NO: 67). In some embodiments, the aldehyde dehydrogenase has an amino acid sequence that is at least 95% identical to the amino acid sequence of SEQ ID NO: 67 (e.g., an amino acid sequence that is 95%, 96%, 97%, 98%, 99%, or 100% identical to SEQ ID NO: 67). In some embodiments, the aldehyde dehydrogenase synthase has the amino acid sequence of SEQ ID NO: 67. In some embodiments, the pyruvate decarboxylase has an amino acid sequence that is at least 90% identical to the amino acid sequence of SEQ ID NO: 65 (e.g., an amino acid sequence that is 90%, 91%, 92%, 93%, 94%, 95%, 96%, 97%, 98%, 99%, or 100% identical to SEQ ID NO: 65). In some embodiments, the pyruvate decarboxylase has an amino acid sequence that is at least 95% identical to the amino acid sequence of SEQ ID NO: 65 (e.g., an amino acid sequence that is 95%, 96%, 97%, 98%, 99%, or 100% identical to SEQ ID NO: 65). In some embodiments, the pyruvate decarboxylase has the amino acid sequence of SEQ ID NO: 65.

In some embodiments, expression of the one or more heterologous nucleic acids is regulated by an exogenous agent. In some embodiments, the exogenous agent decreases production of the cannabinoid. In some embodiments, the exogenous agent increases production of the cannabinoid. In some embodiments, the exogenous agent is galactose and expression of at least one of the heterologous nucleic acids is under the control of a GAL promoter. In some embodiments, expression of at least one of the heterologous nucleic acids is under the control of a galactose-responsive promoter. In some embodiments, expression of at least one of the heterologous nucleic acids is under the control of a maltose-responsive promoter. In some embodiments, expression of at least one of the heterologous nucleic acids is under the control of a combination of both a galactose-responsive promoter and a maltose-responsive promoter.

In another aspect, the disclosure features a method of producing a cannabinoid by culturing a population of genetically modified host cells of any of the above aspects or embodiments of the disclosure in a culture medium under conditions suitable for the host cells to produce the cannabinoid. In some embodiments, the culture medium contains less than 3 mM hexanoic acid (e.g., from 1 nM to 2.9 mM hexanoic acid, from 10 nM to 2.9 mM hexanoic acid, from 100 nM to 2.9 mM hexanoic acid, or from 1 μM to 2.9 mM hexanoic acid) hexanoic acid.

In another aspect, the disclosure features a fermentation composition comprising: a population of genetically modified yeast cells comprising the host cell of any of the above aspects or embodiments of the disclosure and a culture medium comprising one or more cannabinoids produced from the yeast cells.

In another aspect, the disclosure features a method of recovering one or more cannabinoids from the fermentation composition, the method comprising separating at least a portion of the population of genetically modified yeast cells from the culture medium; contacting the separated host cells with a wash liquid; and removing the wash liquid from the separated host cells.

In another aspect, the disclosure features a method of producing a cannabinoid including culturing the mixture of any of the above aspects or embodiments of the disclosure under conditions suitable for the host cells to produce the cannabinoid.

In another aspect, the disclosure features a fermentation composition containing a mixture of any of the above aspects or embodiments of the disclosure.

In another aspect, the disclosure features a non-naturally occurring CBGaS enzyme capable of producing CBGA and at least one additional cannabinoid selected from SCBGA, CBGOA, SCBGOA, CBGVA, SCBGVA, CBGXA, and SCBGXA.

In another aspect, the disclosure features a non-naturally occurring CBGaS enzyme capable of accepting, as a substrate, olivetolic acid and at least one additional precursor selected from orsellinic acid, divarinolic acid, and 2,4-dihydroxy-6-phenylethylbenzoic acid.

In another aspect, the disclosure features a non-naturally occurring CBGaS enzyme capable of catalyzing:
(a) conversion of olivetolic acid to cannabigerolic acid (CBGA) in the presence of GPP and/or to sesquicannabigerolic acid (SCBGA) in the presence of FPP; and/or
(b) conversion of orsellinic acid to cannabigerorcinic acid (CBGOA) in the presence of GPP and/or to sesquicannabigerorcinic acid (SCBGOA) in the presence of FPP; and/or
(c) conversion of divarinolic acid to cannabigerovarinic acid (CBGVA) in the presence of GPP and/or to sesquicannabigerovarinic acid (SCBGVA) in the presence of FPP; and/or
(d) conversion of 2,4-dihydroxy-6-phenylethylbenzoic acid to 3-geranyl-2,4-dihydroxy-6-phenylethylbenzoic acid (CBGXA) in the presence of GPP and/or to 3-farnesyl-2,4-dihydroxy-6-phenylethylbenzoic acid (SCBGXA) in the presence of FPP.

In another aspect, the disclosure features a CBGaS enzyme (e.g., a non-naturally occurring CBGaS enzyme) having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 55, SEQ ID NO:56, SEQ ID NO:57, or SEQ ID NO: 58. In some embodiments, the CBGaS comprises one or more amino acid substitutions relative to the amino acid sequence of SEQ ID NO: 55 selected from M88I, V133I, S141Y, Y319L, and L324F.

In some embodiments of any of the foregoing aspects, the CBGaS has the amino acid substitution M88I relative to the amino acid sequence of SEQ ID NO: 55. In some embodiments, the CBGaS has the amino acid substitution V133I relative to the amino acid sequence of SEQ ID NO: 55. In some embodiments, the CBGaS has the amino acid substitution S141Y relative to the amino acid sequence of SEQ ID NO: 55. In some embodiments, the CBGaS has the amino acid substitution Y319L relative to the amino acid sequence of SEQ ID NO: 55. In some embodiments, the CBGaS has the amino acid substitution L324F relative to the amino acid sequence of SEQ ID NO: 55.

In some embodiments, the CBGaS enzyme has an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 56, wherein the CBGaS comprises one or more amino acid substitutions relative to the amino acid sequence of SEQ ID NO: 56 selected from P7K, P7T, T11T, H49C, M83V, A89A, N93V, A131G, V149F, A176V, R196F, T202A, V242L, T248A, C249F, A257Y, A257F, V262L, N264Y, N264F, L276T, L276P, A279C, A279S, A282P, N309F, M311L, S312L, Y319L, I324E, I324K, L325P, and L325A.

In some embodiments of any of the foregoing aspects, the CBGaS has the amino acid substitution P7K or P7T relative to the amino acid sequence of SEQ ID NO: 56. In some embodiments, the CBGaS has the amino acid substitution T11T relative to the amino acid sequence of SEQ ID NO: 56. In some embodiments, the CBGaS has the amino acid substitution H49C relative to the amino acid sequence of SEQ ID NO: 56. In some embodiments, the CBGaS has the amino acid substitution M83V relative to the amino acid sequence of SEQ ID NO: 56. In some embodiments, the CBGaS has the amino acid substitution A89A relative to the amino acid sequence of SEQ ID NO: 56. In some embodiments, the CBGaS has the amino acid substitution N93V relative to the amino acid sequence of SEQ ID NO: 56. In some embodiments, the CBGaS has the amino acid substitution A131G relative to the amino acid sequence of SEQ ID NO: 56. In some embodiments, the CBGaS has the amino acid substitution V149F relative to the amino acid sequence of SEQ ID NO: 56. In some embodiments, the CBGaS has the amino acid substitution A176V relative to the amino acid sequence of SEQ ID NO: 56. In some embodiments, the CBGaS has the amino acid substitution R196F relative to the amino acid sequence of SEQ ID NO: 56. In some embodiments, the CBGaS has the amino acid substitution T202A relative to the amino acid sequence of SEQ ID NO: 56. In some embodiments, the CBGaS has the amino acid substitution V242L relative to the amino acid sequence of SEQ ID NO: 56. In some embodiments, the CBGaS has the amino acid substitution T248A relative to the amino acid sequence of SEQ ID NO: 56. In some embodiments, the CBGaS has the amino acid substitution C249F relative to the amino acid sequence of SEQ ID NO: 56. In some embodiments, the CBGaS has the amino acid substitution A257Y or A257F relative to the amino acid sequence of SEQ ID NO: 56. In some embodiments, the CBGaS has the amino acid substitution V262L relative to the amino acid sequence of SEQ ID NO: 56. In some embodiments, the CBGaS has the amino acid substitution N264Y or N264F relative to the amino acid sequence of SEQ ID NO: 56. In some embodiments, the CBGaS has the amino acid substitution L276T or L276P relative to the amino acid sequence of SEQ ID NO: 56. In some embodiments, the CBGaS has the amino acid substitution A279C or A279S relative to the amino acid sequence of SEQ ID NO: 56. In some embodiments, the CBGaS has the amino acid substitution A282P relative to the amino acid sequence of SEQ ID NO: 56. In some embodiments, the CBGaS has the amino acid substitution N309F relative to the amino acid sequence of SEQ ID NO: 56. In some embodiments, the CBGaS has the amino acid substitution M311L relative to the amino acid sequence of SEQ ID NO: 56. In some embodiments, the CBGaS has the amino acid substitution S312L relative to the amino acid sequence of SEQ ID NO: 56. In some embodiments, the CBGaS has the amino acid substitution Y319L relative to the amino acid sequence of SEQ ID NO: 56. In some embodiments, the CBGaS has the amino acid substitution I324E or I324K relative to the amino acid sequence of SEQ ID NO: 56. In some embodiments, the CBGaS has the amino acid substitution L325P or L325A relative to the amino acid sequence of SEQ ID NO: 56.

In another aspect, the disclosure features a non-naturally occurring CBGaS enzyme having an amino acid sequence that is at least 90% identical (e.g., at least 91%, at least 92%, at least 93%, at least 94%, at least 95%, at least 96%, at least 97%, at least 98%, at least 99%, or 100% identical) to the amino acid sequence of SEQ ID NO: 63.

In another aspect, the disclosure features a CBGaS enzyme (e.g., a non-naturally occurring CBGaS enzyme) having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 63, wherein the CBGaS has one or more amino acid substitutions relative to the amino acid sequence of SEQ ID NO: 63 selected from I109T, F119L, S245L, S247Y, M270T, C280L, S295D, V314L, A324F, and S361I.

In some embodiments of any of the foregoing aspects, the CBGaS has the amino acid substitution I109T relative to the amino acid sequence of SEQ ID NO: 63. In some embodiments, the CBGaS has the amino acid substitution F119L relative to the amino acid sequence of SEQ ID NO: 63. In some embodiments, the CBGaS has the amino acid substitution S245L relative to the amino acid sequence of SEQ ID NO: 63. In some embodiments, the CBGaS has the amino acid substitution S247Y relative to the amino acid sequence of SEQ ID NO: 63. In some embodiments, the CBGaS has the amino acid substitution M270T relative to the amino acid sequence of SEQ ID NO: 63. In some embodiments, the CBGaS has the amino acid substitution C280L relative to the amino acid sequence of SEQ ID NO: 63. In some embodiments, the CBGaS has the amino acid substitution S295D relative to the amino acid sequence of SEQ ID NO: 63. In some embodiments, the CBGaS has the amino acid substitution V314L relative to the amino acid sequence of SEQ ID NO: 63. In some embodiments, the CBGaS has the amino acid substitution A324F relative to the amino acid sequence of SEQ ID NO: 63. In some embodiments, the CBGaS has the amino acid substitution S361I relative to the amino acid sequence of SEQ ID NO: 63.

In another aspect, the disclosure features a non-naturally occurring CBGaS enzyme having an amino acid sequence that is at least 90% identical (e.g., at least 91%, at least 92%, at least 93%, at least 94%, at least 95%, at least 96%, at least 97%, at least 98%, at least 99%, or 100% identical) to the amino acid sequence of SEQ ID NO: 64.

In another aspect, the disclosure features a CBGaS enzyme (e.g., a non-naturally occurring CBGaS enzyme) having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 64, wherein the CBGaS has one or more amino acid substitutions relative to the amino acid sequence of SEQ ID NO: 64 selected from M275S, M275T, T276C, T276F, K291H, V292Y, V292H, V292F, G310C, F314N, A331C, A331T, and A347I.

In some embodiments of any of the foregoing aspects, the CBGaS has the amino acid substitution M275S or M275T relative to the amino acid sequence of SEQ ID NO: 64. In some embodiments, the CBGaS has the amino acid substitution T276C or T276F relative to the amino acid sequence of SEQ ID NO: 64. In some embodiments, the CBGaS has the amino acid substitution K291H relative to the amino acid sequence of SEQ ID NO: 64. In some embodiments, the CBGaS has the amino acid substitution V292Y, V292H, or V292F relative to the amino acid sequence of SEQ ID NO: 64.

In some embodiments, the CBGaS has the amino acid substitution G310C relative to the amino acid sequence of SEQ ID NO: 64. In some embodiments, the CBGaS has the amino acid substitution F314N relative to the amino acid sequence of SEQ ID NO: 64. In some embodiments, the CBGaS has the amino acid substitution A331C or A331T relative to the amino acid sequence of SEQ ID NO: 64. In some embodiments, the CBGaS has the amino acid substitution A347 I relative to the amino acid sequence of SEQ ID NO: 64.

In another aspect, the disclosure features an OAC enzyme having an amino acid sequence that is at least 90% identical (e.g., at least 91%, at least 92%, at least 93%, at least 94%, at least 95%, at least 96%, at least 97%, at least 98%, at least 99%, or 100% identical) to the amino acid sequence of any one of SEQ ID NOs: 45-52.

In another aspect, the disclosure features an OAC enzyme (e.g., a non-naturally occurring OAC enzyme) having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 44, wherein the OAC has one or more amino acid substitutions relative to the amino acid sequence of SEQ ID NO: 44 selected from A2S, L9I, K12S, E14S, F23L, V28L, T47R, Q48R, K49R, S87H, F88Y, and L92Y.

In some embodiments of any of the foregoing aspects, the OAC has the amino acid substitution A2S relative to the amino acid sequence of SEQ ID NO: 44. In some embodiments, the OAC has the amino acid substitution L91 relative to the amino acid sequence of SEQ ID NO: 44. In some embodiments, the OAC has the amino acid substitution K12S relative to the amino acid sequence of SEQ ID NO: 44. In some embodiments, the OAC has the amino acid substitution E14S relative to the amino acid sequence of SEQ ID NO: 44. In some embodiments, the OAC has the amino acid substitution F23L relative to the amino acid sequence of SEQ ID NO: 44. In some embodiments, the OAC has the amino acid substitution V28L relative to the amino acid sequence of SEQ ID NO: 44. In some embodiments, the OAC has the amino acid substitution T47R relative to the amino acid sequence of SEQ ID NO: 44. In some embodiments, the OAC has the amino acid substitution Q48R relative to the amino acid sequence of SEQ ID NO: 44. In some embodiments, the OAC has the amino acid substitution K49R relative to the amino acid sequence of SEQ ID NO: 44. In some embodiments, the OAC has the amino acid substitution S87H relative to the amino acid sequence of SEQ ID NO: 44. In some embodiments, the OAC has the amino acid substitution F88Y relative to the amino acid sequence of SEQ ID NO: 44. In some embodiments, the OAC has the amino acid substitution L92Y relative to the amino acid sequence of SEQ ID NO: 44.

In another aspect, the disclosure features a nucleic acid encoding the enzyme of any one of the foregoing aspects or embodiments of the disclosure. In another aspect, the disclosure features a host cell comprising the nucleic acid, such as a yeast cell or yeast strain. In some embodiments, the yeast cell is S. cerevisiae, among other possible options described herein.

### BRIEF DESCRIPTION OF THE DRAWINGS

FIG. 1 shows a portion of the cannabinoid biosynthetic pathway referenced herein.

FIG. 2 shows the amount of olivetolic acid produced by 23 proteins from a diversity library, as is described in further detail in the working examples, below. The candidates produced olivetolic acid at 0.21- to 1.27-fold the amount of Cs.AAE.

FIG. 3 shows the amount of olivetolic acid produced by 10 proteins identified from the subsequent natural diversity, which possessed TKS activity that surpassed the activity of the TKS from Cannabis sativa.

FIG. 4 shows the amount of PDAL, HTAL, and olivetol produced from 17 enzymes that were identified during screening to produce olivetolic acid.

FIG. 5 shows the mechanism of the TKS enzyme. A TKS enzyme can directly catalyze formation of PDAL and HTAL, in addition to the tetraketide-CoA intermediate. Within the screening strain, the tetraketide-CoA can either be converted to olivetolic acid by the OAC enzyme or converted to olivetol spontaneously. All four intermediates directly result from TKS enzymatic activity.

FIG. 6 shows the amount of olivetolic acid produced by 8 unique proteins, each containing at least eight amino acid point mutations, that were found to possess more than double the OAC activity of Cs.OAC.

FIG. 7 shows the amount of cannabigerolic acid produced relative to the production of cannabigerolic acid produced by Cs.PT4 by 3 proteins identified in the subsequent natural diversity library.

FIG. 8 shows the structures of cannabigerolic acid (CBGA) and sesquicannabigerolic acid (SCBGA).

FIG. 9 shows a representation of the library of chimeras constructed from Cs.PT4-T and one homolog.

FIG. 10 shows the amount of CBGA and SCBGA produced by the chimeras relative to the production of CBGA and SCBGA produced by Cs.PT4.

FIG. 11. shows a schematic depiction of the DNA landing pad used to facilitate homologous recombination into the yeast screening strains containing an upstream locus, a promoter, a F-Cph1 cut site, a terminator, and a downstream locus.

### DEFINITIONS

As used herein the singular forms “a,” “an,” and, “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the terms “acyl activating enzyme,” “AAE enzyme,” “AAE,” and the like are used interchangeably and refer to an enzyme that catalyzes the activation of a carboxylic acid as a part of the cannabinoid biosynthetic pathway. Exemplary AAE enzymes of the disclosure generate hexanoate from hexanoyl-CoA. Exemplary AAE enzymes of the disclosure include those having the amino acid sequence of any one of SEQ ID NOs: 1-24 or an amino acid sequence that is at least 70% identical (e.g., at least 75%, 80%, 85%, 86%, 87%, 88%, 89%, 90%, 91%, 92%, 93%, 94%, 95%, 96%, 97%, 98%, or 99% identical) thereto.

As used herein, the term “cannabinoid” refers to a chemical substance that binds or interacts with a cannabinoid receptor (for example, a human cannabinoid receptor) and includes, without limitation, chemical compounds such endocannabinoids, phytocannabinoids, and synthetic cannabinoids. Synthetic compounds are chemicals made to mimic phytocannabinoids which are naturally found in the cannabis plant (e.g., Cannabis sativa), including but not limited to cannabigerols (CBG), cannabichromens (CBC), cannabidiol (CBD), tetrahydrocannabinol (THC), cannabinol (CBN), cannabinodiol (CBDL), cannabicyclol (CBL), cannabielsoin (CBE), and cannabitriol (CBT).

As used herein, the term “capable of producing” refers to a host cell which is genetically modified to include the enzymes necessary for the production of a given compound in accordance with a biochemical pathway that produces the compound. For example, a cell (e.g., a yeast cell) “capable of producing” a cannabinoid is one that contains the enzymes necessary for production of the cannabinoid according to the cannabinoid biosynthetic pathway.

As used herein, the term “conservatively modified variants” refers to a nucleic acid or amino acid sequences that are substantially identical to a reference. With respect to particular nucleic acid sequences, conservatively modified variants refer to those nucleic acids which encode identical or essentially identical amino acid sequences, or where the nucleic acid does not encode an amino acid sequence to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, at pH 7, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu and Ile; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. The following six groups each contain amino acids that further provide illustrative conservative substitutions for one another. 1) Ala, Ser, Thr; 2) Asp, Glu; 3) Asn, Gln; 4) Arg, Lys; 5) Ile, Leu, Met, Val; and 6) Phe, Try, and Trp (see, e.g., Creighton, Proteins (1984)).

As used herein, the terms “cannabigerolic acid synthase,” “CBGaS enzyme,” “CBGaS,” and the like are used interchangeably and refer to a prenyltransferase capable of utilizing, for example, GPP or FPP, to convert a precursor, such as olivetolic acid, orsellinic acid, divarinolic acid, or 2,4-dihydroxy-6-phenylethylbenzoic acid, to a cannabinoid, such as cannabigerolic acid (CBGA), cannabigerol (CBG), sesquicannabigerolic acid (SCBGA), cannabigerorcinic acid (CBGOA), sesquicannabigerorcinic acid (SCBGOA), cannabigerovarinic acid (CBGVA), sesquicannabigerovarinic acid (SCBGVA), 3-geranyl-2,4-dihydroxy-6-phenylethylbenzoic acid (CBGXA), or 3-farnesyl-2,4-dihydroxy-6-phenylethylbenzoic acid (SCBGXA). Exemplary CBGaS enzymes of the disclosure include those having the amino acid sequence of any one of SEQ ID NOs: 55-58, 63, and 64, or an amino acid sequence that is at least 70% identical (e.g., at least 75%, 80%, 85%, 86%, 87%, 88%, 89%, 90%, 91%, 92%, 93%, 94%, 95%, 96%, 97%, 98%, or 99% identical) thereto.

As used herein, the term “endogenous” refers to a substance or process that can occur naturally in a host cell. In contrast, the term “exogenous” refers a substance or compound that originated outside an organism or cell. The exogenous substance or compound can retain its normal function or activity when introduced into an organism or host cell described herein.

The term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As explained herein, these substantially identical variants are specifically covered by reference to a specific nucleic acid sequence. One example of an expression cassette is a polynucleotide construct that contains a polynucleotide sequence encoding a polypeptide for use in the invention operably linked to a promoter, e.g., its native promoter, where the expression cassette is introduced into a heterologous microorganism. In some embodiments, an expression cassette contains a polynucleotide sequence encoding a polypeptide of the invention where the polynucleotide that is targeted to a position in the genome of a microorganism such that expression of the polynucleotide sequence is driven by a promoter that is present in the microorganism.

As used herein, the term “fermentation composition” refers to a composition which contains genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

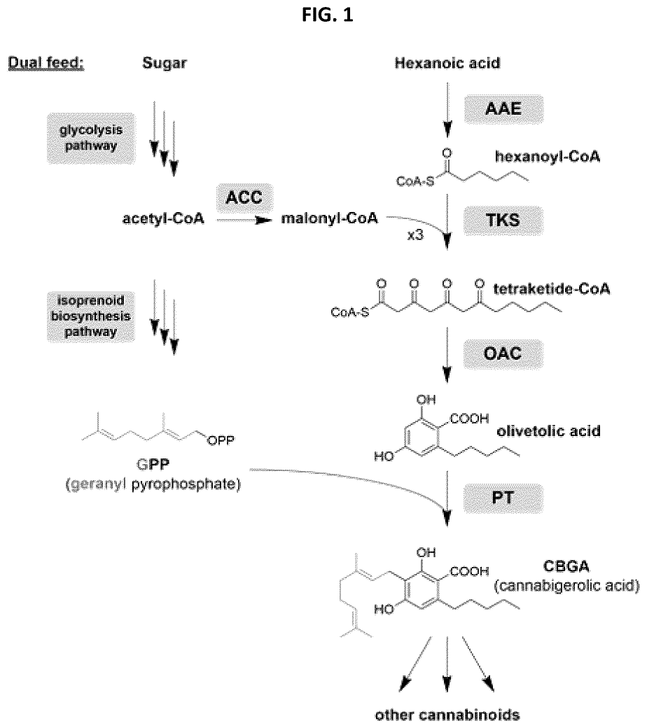

A “genetic pathway” or “biosynthetic pathway” as used herein refer to a set of at least two different coding sequences, where the coding sequences encode enzymes that catalyze different parts of a synthetic pathway to form a desired product (e.g., a cannabinoid). In a genetic pathway a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway. An example of a cannabinoid synthetic pathway is shown in FIG. 1.

As used herein, the term “genetic switch” refers to one or more genetic elements that allow controlled expression of enzymes, e.g., enzymes that catalyze the reactions of cannabinoid biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “genetically modified” denotes a host cell that contains a heterologous nucleotide sequence. The genetically modified host cells described herein typically do not exist in nature.

As used herein, the terms “geranyl pyrophosphate synthase,” “GPP synthase,” “GPPS enzyme,” “GPPS,” and the like are used interchangeably and refer to a prenyltransferase enzyme capable of producing an intermediate in the isoprenoid biosynthesis pathway, such as geranyl pyrophosphate (GPP) or farnesyl pyrophosphate (FPP).

As used herein, the term “heterologous” refers to what is not normally found in nature. The term “heterologous compound” refers to the production of a compound by a cell that does not normally produce the compound, or to the production of a compound at a level not normally produced by the cell. For example, a cannabinoid can be a heterologous compound.

The term “heterologous compound” refers to the production of a compound by a cell that does not normally produce the compound, or to the production of a compound at a level at which it is not normally produced by the cell.

As used herein, the phrase “heterologous enzyme” refers to an enzyme that is not normally found in a given cell in nature. The term encompasses an enzyme that is: (a) exogenous to a given cell (i.e., encoded by a nucleotide sequence that is not naturally present in the host cell or not naturally present in a given context in the host cell); and (b) naturally found in the host cell (e.g., the enzyme is encoded by a nucleotide sequence that is endogenous to the cell) but that is produced in an unnatural amount (e.g., greater or lesser than that naturally found) in the host cell.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “introducing” in the context of introducing a nucleic acid or protein into a host cell refers to any process that results in the presence of a heterologous nucleic acid or polypeptide inside the host cell. For example, the term encompasses introducing a nucleic acid molecule (e.g., a plasmid or a linear nucleic acid) that encodes the nucleic acid of interest (e.g., an RNA molecule) or polypeptide of interest and results in the transcription of the RNA molecules and translation of the polypeptides. The term also encompasses integrating the nucleic acid encoding the RNA molecules or polypeptides into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein, the term “medium” refers to culture medium and/or fermentation medium.

The terms “modified,” “recombinant” and “engineered,” when used to modify a host cell described herein, refer to host cells or organisms that do not exist in nature, or express compounds, nucleic acids or proteins at levels that are not expressed by naturally occurring cells or organisms.

As used herein, the term “non-naturally occurring” refers to a substance (e.g., a protein, such as an enzyme described herein), that is not produced by an organism (e.g., yeast, such as a yeast strain described herein) without human intervention. Exemplary non-naturally occurring enzymes of the disclosure include the modified CBGaS and OAC enzymes described herein, which contain one or more amino acid substitutions relative to a reference enzyme that is naturally occurring.

As used herein, the terms “olivetolic acid cyclase,” “OAC enzyme,” “OAC,” and the like are used interchangeably and refer to an enzyme that catalyzes the cyclization of tetraketide-CoA, thereby generating olivetolic acid, as part of the cannabinoid biosynthetic pathway. Exemplary OAC enzymes of the disclosure include those having the amino acid sequence of any one of SEQ ID NOs: 45-52 or an amino acid sequence that is at least 70% identical (e.g., at least 75%, 80%, 85%, 86%, 87%, 88%, 89%, 90%, 91%, 92%, 93%, 94%, 95%, 96%, 97%, 98%, or 99% identical) thereto.

As used herein, the phrase “operably linked” refers to a functional linkage between nucleic acid sequences such that the linked promoter and/or regulatory region functionally controls expression of the coding sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally-derived nucleic acid that is capable of activating, increasing or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

As used herein, the terms “tetraketide synthase,” “TKS enzyme,” “TKS,” and the like are used interchangeably and refer to an enzyme that is capable of producing tetraketide-CoA from a hexanoyl-CoA precursor in the presence of malonyl-CoA. Exemplary TKS enzymes of the disclosure include those having the amino acid sequence of any one of SEQ ID NOs: 25-43 or an amino acid sequence that is at least 70% identical (e.g., at least 75%, 80%, 85%, 86%, 87%, 88%, 89%, 90%, 91%, 92%, 93%, 94%, 95%, 96%, 97%, 98%, or 99% identical) thereto.

The term “yield” refers to production of a compound by a host cell, expressed as the amount of compound produced per amount of carbon source consumed by the host cell, by weight.

### DETAILED DESCRIPTION OF THE INVENTION

The present disclosure features host cells capable of producing a cannabinoid and methods for genetically modifying a host cell to be capable of producing a cannabinoid. The genetically modified host cells include heterologous nucleic acids that independently encode an acyl activating enzyme (AAE), and/or a tetraketide synthase (TKS), and/or a cannabigerolic acid synthase (CBGaS), and/or an olivetolic acid cyclase (OAC). Provided herein are enzymes that have been identified to have AAE, TKS, CBGaS, or OAC activity, wherein in some embodiments the enzyme identified was found to have greater activity in comparison to the Cannabis sativa wild-type AAE, TKS, CBGaS, or OAC. The AAE, TKS, CBGaS, and OAC are all enzymes part of a heterologous biosynthetic pathway for cannabinoid synthesis. The heterologous biosynthetic pathway can be differentially regulated by one or more exogenous agents.

### Cannabinoid Pathway

In an aspect, the host cell includes a heterologous genetic pathway that produces a cannabinoid or a precursor of a cannabinoid. The cannabinoid biosynthetic pathway may begin with hexanoic acid as the substrate for an acyl activating enzyme (AAE) to produce hexanoyl-CoA, which is used as the substrate of a tetraketide synthase to produce tetraketide-CoA, which is used by an olivetolic acid cyclase (OAC) to produce olivetolic acid, which is then used to produce a cannabigerolic acid by a geranyl pyrophosphate (GPP) synthase and a cannabigerolic acid synthase (CBGaS) as shown in FIG. 1. In some embodiments, the cannabinoid precursor that is produced is a substrate in the cannabinoid pathway (e.g., hexanoate or olivetolic acid). In some embodiments, the precursor is a substrate for an AAE, a TKS, an OAC, a CBGaS, or a GPP synthase. In some embodiments, the precursor, substrate, or intermediate in the cannabinoid pathway is hexanoate, olivetol, or olivetolic acid. In some embodiments, the precursor is hexanoate. In some embodiments, the host cell does not contain the precursor, substrate or intermediate in an amount sufficient to produce the cannabinoid or a precursor of the cannabinoid. In some embodiments, the host cell does not contain hexanoate at a level or in an amount sufficient to produce the cannabinoid in an amount over 10 mg/L. In some embodiments, the heterologous genetic pathway encodes at least one enzyme selected from the group consisting of an AAE, a TKS, an OAC, a CBGaS, or a GPP synthase. In some embodiments, the genetically modified host cell includes an AAE, TKS, OAC, CBGaS, and a GPP synthase. The cannabinoid pathway is described in Keasling et al. (WO 2018/200888).

### Acyl Activating Enzymes

Some embodiments concern a host cell that includes a heterologous AAE such that the host cell is capable of producing a cannabinoid. The AAE may be from Cannabis sativa or may be an enzyme from another plant or fungal source which has been shown to have AAE activity in the cannabinoid biosynthetic pathway, resulting in the production of the cannabinoid precursor olivetolic acid. In some embodiments, the heterologous AAE may have greater activity compared to the AAE from Cannabis sativa (SEQ ID NO: 6).

In some embodiments, the host cell contains a heterologous nucleic acid that encodes an AAE having an amino acid sequence that is at least 90% identical to the amino acid sequence of any one of SEQ ID NOS: 1-24 (e.g., an amino acid sequence that is 90%, 91%, 92%, 93%, 94%, 95%, 96%, 97%, 98%, 99%, or 100% identical to any one of SEQ ID NOS: 1-24). For example, the AAE may have an amino acid sequence that is at least 90% identical to the amino acid sequence of any one of SEQ ID NO: 1-5 and 7-24. In some embodiments, the AAE has an amino acid sequence that is at least 95% identical to the amino acid sequence of any one of SEQ ID NO: 1-5 and 7-24 (e.g., an amino acid sequence that is 95%, 96%, 97%, 98%, 99%, or 100% identical to any one of SEQ ID NO: 1-5 and 7-24). In some embodiments, the AAE has the amino acid sequence of any one of SEQ ID NO: 1-5 and 7-24. In some embodiments, the host cell contains a heterologous nucleic acid that encodes an AAE having an amino acid sequence that is at least 90% identical to the amino acid sequence of any one of SEQ ID NO: 1-4 (e.g., an amino acid sequence that is 90%, 91%, 92%, 93%, 94%, 95%, 96%, 97%, 98%, 99%, or 100% identical to any one of SEQ ID NO: 1-4). In some embodiments, the AAE has an amino acid sequence that is at least 95% identical to the amino acid sequence of any one of SEQ ID NO: 1-4 (e.g., an amino acid sequence that is 95%, 96%, 97%, 98%, 99%, or 100% identical to any one of SEQ ID NO: 1-4). In some embodiments, the AAE has the amino acid sequence of any one of SEQ ID NO: 1-4.

### Tetraketide Synthase Enzymes

Some embodiments concern a host cell that includes a heterologous TKS such that the host cell is capable of producing a cannabinoid. A TKS uses the hexanoyl-CoA precursor to generate tetraketide-CoA. The TKS may be from Cannabis sativa or may be an enzyme from another plant or fungal source which has been shown to have TKS activity in the cannabinoid biosynthetic pathway, resulting in the production of the cannabinoid precursor olivetolic acid. In some embodiments, the heterologous TKS may have greater activity compared to the TKS from Cannabis sativa (SEQ ID NO: 26).

In some embodiments, the host cell contains a heterologous nucleic acid that encodes a TKS having an amino acid sequence that is at least 90% identical to the amino acid sequence of any one of SEQ ID NOS: 25 and 27-43 (e.g., an amino acid sequence that is 90%, 91%, 92%, 93%, 94%, 95%, 96%, 97%, 98%, 99%, or 100% identical to any one of SEQ ID NOS: 25 and 27-43). In some embodiments, the TKS has an amino acid sequence that is at least 95% identical to the amino acid sequence of any one of SEQ ID NOS: 25 and 27-43 (e.g., an amino acid sequence that is 95%, 96%, 97%, 98%, 99%, or 100% identical to any one of SEQ ID NOS: 25 and 27-43). In some embodiments, the TKS has the amino acid sequence of any one of SEQ ID NOS: 25 and 27-43. In some embodiments, the host cell contains a heterologous nucleic acid that encodes a TKS having an amino acid sequence that is at least 90% identical to the amino acid sequence of any one of SEQ ID NOS: 25 and 27-39 (e.g., an amino acid sequence that is 90%, 91%, 92%, 93%, 94%, 95%, 96%, 97%, 98%, 99%, or 100% identical to any one of SEQ ID NOS: 25 and 27-39). In some embodiments, the TKS has an amino acid sequence that is at least 95% identical to the amino acid sequence of any one of SEQ ID NOS: 25 and 27-39 (e.g., an amino acid sequence that is 95%, 96%, 97%, 98%, 99%, or 100% identical to any one of SEQ ID NOS: 25 and 27-39). In some embodiments, the TKS has the amino acid sequence of any one of SEQ ID NOS: 25 and 27-39. In some embodiments, the host cell contains a heterologous nucleic acid that encodes a TKS having an amino acid sequence that is at least 90% identical to the amino acid sequence of SEQ ID NO: 25 or 39 (e.g., an amino acid sequence that is 90%, 91%, 92%, 93%, 94%, 95%, 96%, 97%, 98%, 99%, or 100% identical to SEQ ID NO: 25 or 39). In some embodiments, the TKS has an amino acid sequence that is at least 95% identical to the amino acid sequence of SEQ ID NO: 25 or 39 (e.g., an amino acid sequence that is 95%, 96%, 97%, 98%, 99%, or 100% identical to SEQ ID NO: 25 or 39). In some embodiments, the TKS has the amino acid sequence of SEQ ID NO: 25 or 39.

### Cannabigerolic Acid Synthases

Some embodiments concern novel CBGaS enzymes, and/or a host cell that includes a heterologous CBGaS such that the host cell is capable of producing a cannabinoid. In some embodiments, a CBGaS of the disclosure uses an olivetolic acid precursor and GPP or FPP to generate cannabigerolic acid (CBGA) or sesquicannabigerolic acid (SCBGA). In some embodiments, a CBGaS of the disclosure uses an orsellinic acid precursor and GPP or FPP to generate cannabigerorcinic acid (CBGOA) or sesquicannabigerorcinic acid (SCBGOA). In some embodiments, a CBGaS of the disclosure uses a divarinolic acid precursor and GPP or FPP to generate cannabigerovarinic acid (CBGVA) or sesquicannabigerovarinic acid (SCBGVA). In some embodiments, a CBGaS of the disclosure uses 2,4-dihydroxy-6-phenylethylbenzoic acid and GPP or FPP to generate 3-geranyl-2,4-dihydroxy-6-phenylethylbenzoic acid (CBGXA) or 3-farnesyl-2,4-dihydroxy-6-phenylethylbenzoic acid (SCBGXA). The CBGaS may be from Cannabis sativa or may be an enzyme from another plant or fungal source which has been shown to have CBGaS activity in the cannabinoid biosynthetic pathway, resulting in the production of the cannabinoid cannabigerolic acid. In some embodiments, the heterologous CBGaS may have greater activity compared to the TKS from Cannabis sativa (SEQ ID NO: 53).

In some embodiments, the host cell contains a heterologous nucleic acid that encodes a CBGaS having an amino acid sequence that is at least 90% identical to the amino acid sequence of any one of SEQ ID NOS: 55-58, 63, and 64 (e.g., an amino acid sequence that is 90%, 91%, 92%, 93%, 94%, 95%, 96%, 97%, 98%, 99%, or 100% identical to any one of SEQ ID NOS: 55-58, 63, and 64). In some embodiments, the CBGaS has an amino acid sequence that is at least 95% identical to the amino acid sequence of any one of SEQ ID NOS: 55-58, 63, and 64 (e.g., an amino acid sequence that is 95%, 96%, 97%, 98%, 99%, or 100% identical to any one of SEQ ID NOS: 55-58, 63, and 64). In some embodiments, the CBGaS has the amino acid sequence of any one of SEQ ID NOS: 55-58, 63, and 64.

### Olivetolic Acid Cyclase Enzymes

Some embodiments concern a host cell that includes a heterologous OAC such that the host cell is capable of producing a cannabinoid. OAC uses the olivetolic acid precursor and GPP precursor to generate cannabigerolic acid. The OAC may be from Cannabis sativa or may be an enzyme from another plant or fungal source which has been shown to have OAC activity in the cannabinoid biosynthetic pathway, resulting in the production of the cannabinoid cannabigerolic acid. In some embodiments, the heterologous OAC may have greater activity compared to the OAC from Cannabis sativa (SEQ ID NO: 44).

In some embodiments, the host cell contains a heterologous nucleic acid that encodes an OAC having an amino acid sequence that is at least 90% identical to the amino acid sequence of any one of SEQ ID NOS: 45-52 (e.g., an amino acid sequence that is 90%, 91%, 92%, 93%, 94%, 95%, 96%, 97%, 98%, 99%, or 100% identical to any one of SEQ ID NOS: 45-52). In some embodiments, the OAC has an amino acid sequence that is at least 95% identical to the amino acid sequence of any one of SEQ ID NOS: 45-52 (e.g., an amino acid sequence that is 95%, 96%, 97%, 98%, 99%, or 100% identical to any one of SEQ ID NOS: 45-52). In some embodiments, OAC has the amino acid sequence of any one of SEQ ID NOS: 45-52.

### Geranyl Pyrophosphate Synthase

Some embodiments concern a host cell that includes a heterologous GPP synthase such that the host cell is capable of producing a cannabinoid. A GPP synthase uses the product of the isoprenoid biosynthesis pathway precursor to generate cannabigerolic acid together with a prenyltransferase enzyme. The GPP synthase may be from Cannabis sativa or may be an enzyme from another plant or bacterial source which has been shown to have GPP synthase activity in the cannabinoid biosynthetic pathway, resulting in the production of the cannabinoid cannabigerolic acid.

In some embodiments, the host cell contains a heterologous nucleic acid that encodes a GPP synthase having an amino acid sequence that is at least 90% identical to the amino acid sequence of SEQ ID NO: 75 (e.g., an amino acid sequence that is 90%, 91%, 92%, 93%, 94%, 95%, 96%, 97%, 98%, 99%, or 100% identical to SEQ ID NO: 65-75). In some embodiments, the GPP synthase has an amino acid sequence that is at least 95% identical to the amino acid sequence of SEQ ID NO: 75 (e.g., an amino acid sequence that is 95%, 96%, 97%, 98%, 99%, or 100% identical to SEQ ID NO: 65-75). In some embodiments, GPP synthase has the amino acid sequence of SEQ ID NO: 75.

### Chimeric Enzymes

Some embodiments concern a host cell that includes a heterologous protein produced by chimeragenesis such that the host cell is capable of producing a cannabinoid as described in Examples 14 and 15. The techniques of protein chimeragenesis is part of a family of protein engineering techniques referred to as DNA shuffling, recombination, molecular breeding, simply “chimeragenesis,” or other names (Engqvist M K M & Rabe K S, Plant Physiol. 179:3, 2019, 907-917). In chimeragenesis, new protein sequences are constructed by concatenating different parts of two or more homologous proteins, and the resulting proteins may possess properties not found in any of the parents (Otey C R et al., PLOS Biol. 4:5, 2006, e112). While many proteins generated via chimeragenesis may be non-functional due to protein mis-folding, a careful choice of crossover sites between homologous proteins can result in chimeric proteins that are more likely to be folded and functional (Voigt C A et al., Nat. Struct. Biol., 9:7, 2002, 553-558).

In some embodiments, the host cell contains a heterologous nucleic acid that encodes a chimeric CBGaS enzyme. In some embodiments, the parent protein for chimeragenesis includes an amino acid sequence having at least 90% sequence identity to any one of SEQ ID NO: 59-62 (e.g., an amino acid sequence that is 90%, 91%, 92%, 93%, 94%, 95%, 96%, 97%, 98%, 99%, or 100% identical to SEQ ID NO: 59-62). In some embodiments, the parent protein for chimeragenesis includes an amino acid sequence having at least 95% sequence identity to any one of SEQ ID NO: 59-62 (e.g., an amino acid sequence that is 95%, 96%, 97%, 98%, 99%, or 100% identical to SEQ ID NO: 59-62). In some embodiments, the parent protein for chimeragenesis includes an amino acid sequence of SEQ ID NO: 59-62. In some embodiments, the host cell contains a heterologous nucleic acid that encodes a CBGaS enzyme having at least 90% sequence identity to SEQ ID NO: 63 or 64 (e.g., an amino acid sequence that is 90%, 91%, 92%, 93%, 94%, 95%, 96%, 97%, 98%, 99%, or 100% identical to SEQ ID NO: 63 or 64). In some embodiments, the host cell contains a heterologous nucleic acid that encodes a chimeric CBGaS enzyme having at least 95% sequence identity to SEQ ID NO: 63 or 64 (e.g., an amino acid sequence that is 95%, 96%, 97%, 98%, 99%, or 100% identical to SEQ ID NO: 63 or 64). In some embodiments, the host cell contains a heterologous nucleic acid that encodes a CBGaS enzyme having the amino acid sequence of SEQ ID NO: 63 or 64.

In some embodiments, the host cell contains a heterologous nucleic acid that encodes a CBGaS that includes a M88I, V133I, S141Y, Y319L, L324F, V149F, M83V, T202A, N264Y, N264F, A282P, S312L, T11T, L276T, L276P, I324E, H49C, S312L, L325P, I324K, L325A, P7K, R196F, A176V, N309F, P7T, A279C, A279S, A89A, V262L, N93V, A257Y, A131G, A257F, V242L, C249F, M311L, T248A, M83V, I109T, F119L, S245L, S247Y, M270T, S295D, C280L, V314L, A324F, or S361I substitution relative to SEQ ID NO: 54. In some embodiments, the CBGaS enzyme produced by chimeragenesis has increased substrate specificity relative to the wild-type enzyme. In some embodiments, the CBGaS enzyme produced by chimeragenesis has an increased production of cannabigerolic acid relative to the wild-type CBGaS. In some embodiments, the CBGaS enzyme produced by chimeragenesis has a decreased production of sesquicannabigerolic acid relative to the wild-type CBGaS.

### Additional Enzymes

The host cell may further express other heterologous enzymes in addition to the AAE, TKS, CBGaS, OAC, and/or GPP synthase. For example, the host cell may include enzymes that make up the mevalonate biosynthetic pathway. These enzymes may include but are not limited to an acetyl-CoA thiolase, a HMG-COA synthase, a HMG-CoA reductase, a mevalonate kinase, a phosphomevalonate kinase, a mevalonate pyrophosphate decarboxylase, and an IPP:DMAPP isomerase. In some embodiments, the host cell includes a heterologous nucleic acid that encodes the acetyl-CoA thiolase, the HMG-COA synthase, the HMG-COA reductase, the mevalonate kinase, the phosphomevalonate kinase, the mevalonate pyrophosphate decarboxylase, and the IPP:DMAPP isomerase of the mevalonate biosynthesis pathway. In some embodiments, host cell contains a heterologous nucleic acid encoding an acetoacetyl-CoA synthase (AACS) instead of a heterologous nucleic acid encoding an acetyl-CoA thiolase. In some embodiments, the host cell contains a heterologous nucleic acid encoding an acetyl-CoA carboxylase (ACC) instead of a heterologous nucleic acid encoding an acetyl-CoA thiolase.

In some embodiments, the host cell may express heterologous enzymes of the central carbon metabolism. Enzymes of the central carbon metabolism may include an acetyl-CoA synthase, an aldehyde dehydrogenase, and a pyruvate decarboxylase. In some embodiments, the host cell includes heterologous nucleic acids that independently encode an acetyl-CoA synthase, and/or an aldehyde dehydrogenase, and/or a pyruvate decarboxylase. In some embodiments, the acetyl-CoA synthase and the aldehyde dehydrogenase from Saccharomyces cerevisiae, and the pyruvate decarboxylase from Zymomonas mobilis. In some embodiments, the acetyl-CoA synthase has an amino acid sequence that is at least 90% (e.g., at least 91%, 92%, 93%, 94%, 95%, 96%, 97%, 98%, and 99%) identical to the amino acid sequence of SEQ ID NO: 66. In some embodiments, the acetyl-CoA synthase has an amino acid sequence of SEQ ID NO: 66. In some embodiments, the aldehyde dehydrogenase has an amino acid sequence that is at least 90% (e.g., at least 91%, 92%, 93%, 94%, 95%, 96%, 97%, 98%, and 99%) identical to the amino acid sequence of SEQ ID NO: 67. In some embodiments, the aldehyde dehydrogenase has an amino acid sequence of SEQ ID NO: 67. In some embodiments, the pyruvate dehydrogenase has an amino acid sequence that is at least 90% (e.g., at least 91%, 92%, 93%, 94%, 95%, 96%, 97%, 98%, and 99%) identical to the amino acid sequence of SEQ ID NO: 65. In some embodiments, the pyruvate decarboxylase has an amino acid sequence of SEQ ID NO: 65.

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).

Those of skill in the art will recognize that, due to the degenerate nature of the genetic code, a variety of DNA molecules differing in their nucleotide sequences can be used to encode a given enzyme of the disclosure. Any one of the polypeptide sequences disclosed herein may be encoded by DNA molecules of any sequence that encode the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (e.g., Pearson W. R., 1994, Methods in Mol Biol 25:365-89).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed in the host cell. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous ADA genes, proteins, or enzymes, techniques may include, but are not limited to, cloning a gene by PCR using primers based on a published sequence of an ADA gene/enzyme or by degenerate PCR using degenerate primers designed to amplify a conserved region among ADA genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, JGI Phyzome v12.1, BLAST, NCBI RefSeq, UniProt KB, or MetaCYC Protein annotations in the UniProt Knowledgebase may also be used to identify enzymes which have a similar function in addition to the National Center for Biotechnology Information RefSeq database. The candidate gene or enzyme may be identified within the above-mentioned databases in accordance with the teachings herein.

### Modified Host Cells

In one aspect, provided herein are host cells comprising at least one enzyme of the cannabinoid biosynthetic pathway (e.g., AAE, TKS, CBGaS, and OAC). In some embodiments, the cannabinoid biosynthetic pathway contains a genetic regulatory element, such as a nucleic acid sequence, that is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate production of a cannabinoid and provide a carbon source for growth of the host cell. In some embodiments, the exogenous agent is galactose. In some embodiments, the exogenous agent is maltose.

In some embodiments, the genetic regulatory element is a nucleic acid sequence, such as a promoter.

In some embodiments, the genetic regulatory element is a galactose-responsive promoter. In some embodiments, galactose positively regulates expression of the cannabinoid biosynthetic pathway, thereby increasing production of the cannabinoid. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, heterologous genetic pathway contains the galactose-responsive regulatory elements described in Westfall et al. (PNAS (2012) vol. 109: E111-118). In some embodiments, the host cell lacks the gal1 gene and is unable to metabolize galactose, but galactose can still induce galactose-regulated genes.

In some embodiments, the galactose regulation system used to control expression of AAE, and/or, TKS, and/or CBGaS, and/or OAC is re-configured such that it is no longer induced by the presence of galactose. Instead, the genes (e.g., AAE, TKS, CBGaS, or OAC) will be expressed unless repressors, which may be maltose in some strains, are present in the medium.

In some embodiments, the genetic regulatory element is a maltose-responsive promoter. In some embodiments, maltose negatively regulates expression of the cannabinoid biosynthetic pathway, thereby decreasing production of the cannabinoid. In some embodiments, the maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Patent Publication 2016/0177341, which is hereby incorporated by reference. Genetic regulation of maltose metabolism is described in Novak et al., “Maltose Transport and Metabolism in S. cerevisiae,” Food Technol. Biotechnol. 42 (3) 213-218 (2004).

In some embodiments, the heterologous genetic pathway is regulated by a combination of the maltose and galactose regulons.

In some embodiments, the recombinant host cell does not contain, or expresses a very low level of (for example, an undetectable amount), a precursor (e.g., hexanoic acid) required to make the cannabinoid. In some embodiments, the precursor (e.g., hexanoic acid) is a substrate of an enzyme in the cannabinoid biosynthetic pathway.

### Yeast Strains

In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### Mixtures

In another aspect, provided are mixtures of the host cells described herein and a culture medium described herein. In some embodiments, the culture medium contains an exogenous agent described herein. In some embodiments, the culture medium contains an exogenous agent that decreases production of a cannabinoid. In some embodiments, the exogenous agent that decreases production of the heterologous product is maltose. In a particular embodiment, the exogenous agent that decreases production of a cannabinoid is maltose.

In some embodiments, the culture medium contains an exogenous agent that increases production of the cannabinoid. In some embodiments, the exogenous agent that increases production of the cannabinoid is galactose. In some embodiments, the culture medium contains a precursor or substrate required to make the cannabinoid. In some embodiments, the precursor required to make the cannabinoid is hexanoate. In some embodiments, the precursor required to make the cannabinoid is hexanoic acid. In some embodiments, the precursor required to make the cannabinoid is olivetolic acid.

In some embodiments, the culture medium contains an exogenous agent that increases production of the cannabinoid and a precursor or substrate required to make the cannabinoid. In some embodiments, the exogenous agent that increases production of the cannabinoid is galactose, and the precursor or substrate required to make the cannabinoid is hexanoate.

### Methods of Making the Host Cells

In another aspect, provided are methods of making the modified host cells described herein. In some embodiments, the methods include transforming a host cell with the heterologous nucleic acid constructs described herein which encode the proteins expressed by a heterologous genetic pathway described herein. Methods for transforming host cells are described in “Laboratory Methods in Enzymology: DNA”, Edited by Jon Lorsch, Volume 529, (2013); and U.S. Pat. No. 9,200,270 to Hsieh, Chung-Ming, et al., and references cited therein.

### Methods for Producing a Cannabinoid

In another aspect, methods are provided for producing a cannabinoid are described herein. In some embodiments, the method decreases expression of the cannabinoid. In some embodiments, the method includes culturing a host cell comprising at least one enzyme of the cannabinoid biosynthetic pathway described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in less than 0.001 mg/L of cannabinoid or a precursor thereof.

In some embodiments, the method is for decreasing expression of a cannabinoid or precursor thereof. In some embodiments, the method includes culturing a host cell comprising an AAE, and/or a TKS, and/or a CBGaS, and/or an OAC described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in the production of less than 0.001 mg/L of a cannabinoid or a precursor thereof.

In some embodiments, the method increases the expression of a cannabinoid. In some embodiments, the method includes culturing a host cell comprising an AAE, and/or a TKS, and/or a CBGaS, and/or an OAC described herein in a medium comprising the exogenous agent, wherein the exogenous agent increases expression of the cannabinoid. In some embodiments, the exogenous agent is galactose. In some embodiments, the method further includes culturing the host cell with the precursor or substrate required to make the cannabinoid.

In some embodiments, the method increases the expression of a cannabinoid product or precursor thereof. In some embodiments, the method includes culturing a host cell comprising a heterologous cannabinoid pathway described herein in a medium comprising an exogenous agent, wherein the exogenous agent increases the expression of the cannabinoid or a precursor thereof. In some embodiments, the exogenous agent is galactose. In some embodiments, the method further includes culturing the host cell with a precursor or substrate required to make the cannabinoid or precursor thereof. In some embodiments, the precursor required to make the cannabinoid or precursor thereof is hexanoate. In some embodiments, the combination of the exogenous agent and the precursor or substrate required to make the cannabinoid or precursor thereof produces a higher yield of cannabinoid than the exogenous agent alone.

In some embodiments, the cannabinoid or a precursor thereof is cannabidiolic acid (CBDA), cannabidiol (CBD), cannabigerolic acid (CBGA), or cannabigerol (CBG).

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing cannabinoids provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag Gmbh & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of cannabinoids may also occur in these culture conditions, but at undetectable levels (with detection limits being about <0.1 g/l). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 mL/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The culture medium may be supplemented with hexanoic acid or hexanoate as a precursor for the cannabinoid biosynthetic pathway. The hexanoic acid may have a concentration of less than 3 mM hexanoic acid (e.g., from 1 nM to 2.9 mM hexanoic acid, from 10 nM to 2.9 mM hexanoic acid, from 100 nM to 2.9 mM hexanoic acid, or from 1 μM to 2.9 mM hexanoic acid) hexanoic acid.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Transformation of Heterologous Nucleic Acids into Yeast Cells

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) medium at 30° C. with shaking (200 rpm), diluted to an OD₆₀₀ of 0.1 in 100 mL YPD, and grown to an OD₆₀₀ of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000× g) for 30 s, the supernatant was removed, and the cells were resuspended in a transformation mix consisting of 240 μL 50% PEG, 36 μL 1 M lithium acetate, 10 μL boiled salmon sperm DNA, and 74 μL of donor DNA. For transformations that require expression of the endonuclease F-Cph1, the donor DNA included a plasmid carrying the F-Cphl gene expressed under the yeast TDH3 promoter. F-Cphl endonuclease expressed in such a manner cuts a specific recognition site engineered in a host strain to facilitate integration of the target gene of interest. Following a heat shock at 42° C. for 40 min, cells were recovered overnight in YPD medium before plating on selective medium. DNA integration was confirmed by colony PCR with primers specific to the integrations.

### Example 2: Culturing of Yeast

For routine strain characterization in a 96-well-plate format, yeast colonies were picked into a 1.1-mL-per-well capacity 96-well ‘Pre-Culture plate’ filled with 360 μL per well of pre-culture medium. Pre-culture medium consists of Bird Seed Media (BSM, originally described by van Hoek et al., Biotech. and Bioengin., 68, 2000, 517-23) at pH 5.05 with 14 g/L sucrose, 7 g/L maltose, 3.75 g/L ammonium sulfate, and 1 g/L lysine. Cells were cultured at 28° C. in a high capacity microtiter plate incubator shaking at 1000 rpm and 80% humidity for 3 days until the cultures reached carbon exhaustion.

The growth-saturated cultures were sub-cultured by taking 14.4 μL from the saturated cultures and diluting into a 2.2 mL per well capacity 96-well ‘production plate’ filled with 360 μL per well of production medium. Production medium consists of BSM at pH 5.05 with 40 g/L sucrose, 3.75 g/L ammonium sulfate, and 2 mM hexanoic acid. Cells in the production medium were cultured at 30° C. in a high capacity microtiter plate shaker at 1000 rpm and 80% humidity for an additional 3 days prior to extraction and analysis.

### Example 3: Analytical Methods for Product Extraction and Titer Determination

At the conclusion of the incubation of the production plate, methanol was added to each well such that the final concentration is 67% (v/v) methanol. An impermeable seal was added, and the plate was shaken at 1000 rpm for 5 minutes to lyse the cells and extract cannabinoids. The plate was centrifuged for 5 minutes at 2000× g to pellet cell debris. Subsequently, 300 μL of the clarified sample was transferred to an empty 1.1-mL-capacity 96-well plate and sealed with a foil seal. The sample plate was stored at −20° C. until analysis.

Samples for olivetolic acid and CBGA measurements were initially analyzed in high-throughput by mass spectrometer (Agilent 6470-QQQ) with a RapidFire 365 system autosampler with C4 cartridge.

TABLE 1
RapidFire 365 system configuration

| | | |
|---|---|---|
| Pump 1: 0.1% acetic acid in | 0.8 | mL/min |
| water | | |
| Pump 2: 0.1% formic acid in | 1.5 | mL/min |
| acetonitrile | | |
| Pump 3: 0.1% formic acid in | 0.8 | mL/min |
| 40% acetone in water | | |
| State 1: Aspirate | 600 | ms |
| State 2: Load/Wash | 2000 | ms |
| State 3: Extra wash | 500 | ms |
| State 4: Elute | 6000 | ms |
| State 5: Reequilibrate | 1000 | ms |

TABLE 2
Agilent 6470-QQQ MS method configurations

| Ion Source | AJS ESI | |
|---|---|---|
| Time Filtering peak width | 0.02 | min |
| Stop Time | No limit/as pump | |
| Scan Type | MRM | |
| Diverter Valve | To MS | |
| Delta EMV | (+)0/(−)0 | |
| Ion Mode (polarity) | Negative | |
| Gas Temp | 300° C. | |
| Gas Flow | 13 | L/min |
| Nebulizer | 30 | psi |
| Sheath Gas Temp | 30° C. | |
| Sheath Gas Flow | 12 | L/min |
| Negative Capillary V | 3500 | V |

The peak areas from a chromatogram from a mass spectrometer were used to generate the calibration curve using authentic standards. The amounts, in moles, of each compound were generated through external calibration using an authentic standard.

Hit samples from the initial screen were then analyzed for HTAL, PDAL, olivetol, olivetolic acid, CBGA, and SCBGA on a weight per volume basis, by the two methods below. All measurements were performed by reverse phase ultra-high pressure liquid chromatography and ultraviolet detection (UPLC-UV) using Thermo Vanquish Flex Binary UHPLC System with a Vanquish Diode Array Detector HL.

TABLE 3
Mobile Phases and Column Information

| | |
|---|---|
| Mobile Phase A: | 99.9% water + 0.1% Formic Acid, 5 mM |
| | ammonium formate |
| Mobile Phase B: | 99.9% acetonitrile + 0.1% Formic acid |
| Column for | Thermo Scientific Accucore Polar Premium C18 |
| method #1 | 100 mm × 2.1 mm × 2.6 um, Thermo P/N 28026- |
| | 103030 |
| Guard Column for | Thermo Scientific Guard Cartridge, 4 PK, P/N |
| method #1 | 28103014001 |
| Column for | Restek Raptor ARC-18 100 mm × 3.0 mm × 1.8 |
| method #2 | um, Restek P/N 931421E |
| Guard Column for | Restek UltraShield UHPLC PreColumn Filter |
| method #2 | 0.2 um frit, P/N 25809 |

TABLE 4
Mobile Phase Gradient for Method #1

| Time [min] | Flow [mL/min] | % A | % B | Pump Curve |
|---|---|---|---|---|
| 0.00 | 1.2 | 70 | 30 | 5 |
| 1.00 | 1.2 | 20 | 80 | 5 |
| 1.75 | 1.2 | 12.5 | 87.5 | 5 |
| 1.80 | 1.2 | 70 | 30 | 5 |
| 2.1 | 1.2 | 70 | 30 | 5 |

TABLE 5
Isocratic Mobile Phase for Method #2

| Time [min] | Flow [mL/min] | % A | % B | Pump Curve |
|---|---|---|---|---|
| 0.00 | 1.0 | 25 | 75 | 5 |
| 4.00 | 1.0 | 25 | 75 | 5 |

TABLE 6
Column compartment settings

| Parameter: | Method #1 | Method #2 |
|---|---|---|
| Temperature control | On | On |

| | | | | |
|---|---|---|---|---|
| Temperature | 50.0 | C. | 30.0 | C. |
| Ready temp delta | 0.50 | C. | 0.50 | C. |
| Equilibration time | 1.0 | min | 1.0 | min |

| | | |
|---|---|---|
| Thermostatting mode | Still air | Still air |
| Fan Speed | 5 | 5 |

TABLE 7
Detector Settings

| | | |
|---|---|---|
| Parameter: | Method #1 | Method #2 |

| | | | | |
|---|---|---|---|---|
| UV-Vis Channel 1 Wavelength | 270 | nm | 228 | nm |
| Data collection rate | 50.0 | Hz | 5.0 | Hz |
| Response time | 0.10 | s | 1.00 | s |
| Peak width | 0.010 | min | 0.100 | min |

Analytes were identified by retention time compared to an authentic standard. The peak areas were used to generate the linear calibration curve for each analyte.

### Example 4: Generation of the Base Strain for AAE Screening

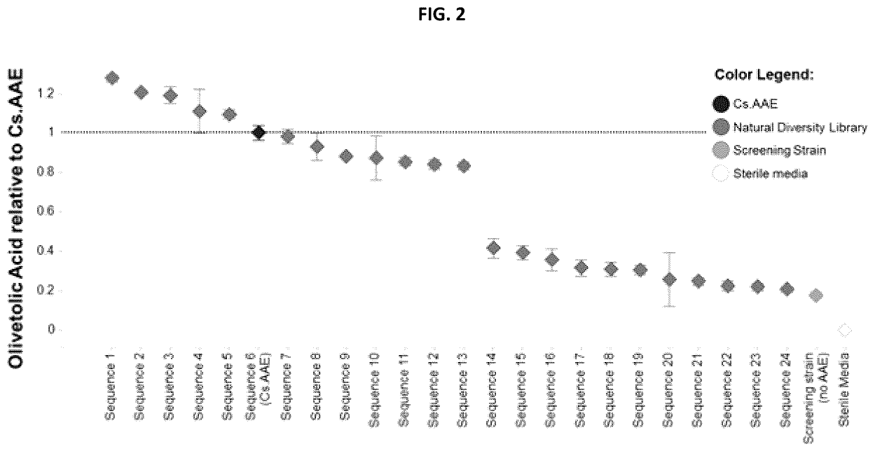

A set of genes for screening for AAE activity was engineered into Saccharomyces cerevisiae in two steps (Table 8). First, constructs were integrated into chromosomal loci to express three genes: a heterologous Zymomonas mobilis PDC gene and two endogenous S. cerevisiae ACS1 and ALD6 genes, all using GAL-regulon promoters. Second, constructs were integrated into chromosomal loci to express TKS and OAC genes from Cannabis sativa (2 and 3 copies, respectively). The resulting strain was capable of producing olivetolic acid in the presence of an AAE enzyme when fed a mixture of sucrose and hexanoic acid, as described in Example 2: Culturing of Yeast. Endogenous yeast metabolism produced a negligible amount of hexanoyl-CoA, which resulted in this strain producing a trace amount of olivetolic acid even in the absence of an exogenous AAE enzyme. This endogenous activity did not interfere with an accurate assessment of proteins with potential AAE activity, as the addition of an exogenous AAE gene to this strain could result in over five times higher amounts of olivetolic acid production (FIG. 2).

TABLE 8
Representation of the cannabinoid pathway in the engineered
S. cerevisiae strain designed for AAE screening.

| Enzyme | SEQ ID NOs | Copy number and Promoter |
|---|---|---|
| Zm.PDC | Sequence 65 | 1 × pGAL7 |
| Sc.ACS1 | Sequence 66 | 1 × pGAL10 |
| Sc.ALD6 | Sequence 67 | 1 × pGAL1 |
| Cs.TKS | Sequence 26 | 2 × pGAL10 |
| Cs.OAC | Sequence 44 | 3 × pGAL1 |

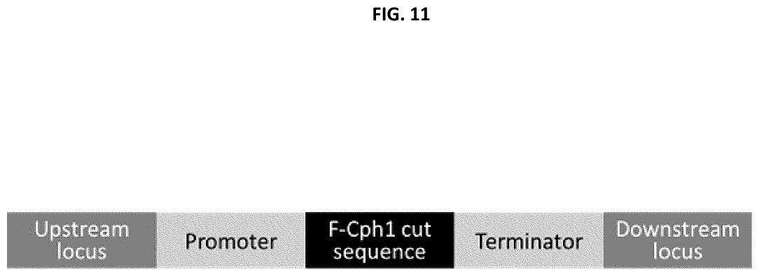

To measure the activity of proteins with potential AAE activity in vivo in S. cerevisiae, a landing pad was introduced into this screening strain, which allows for the rapid insertion of AAE variants (FIG. 11). The landing pad consists of 500 bp of locus-targeting DNA sequences on either end of the construct to the genomic region upstream and downstream of the yeast locus of choice (Upstream locus and Downstream locus), thereby deleting the locus when the landing pad is integrated into the yeast chromosome. Internally, the landing pad contains a promoter which can be GAL1, GAL3 or any other promoter of yeast GAL regulon, and a yeast terminator of choice flanking an endonuclease recognition site (F-Cphl). DNA variants of the AAE library were used to transform the strain along with a plasmid expressing endonuclease F-Cphl, which cuts the recognition sequence, creating a double strand break at the landing pad, and facilitating homologous recombination of the DNA variants at the site. At least six colonies from each transformation were used to screen for AAE activity, using methods described in Example 2: Culturing of Yeast and Example 3: Analytical Methods for Product Extraction and Titer DeterminationExample 3: Analytical Methods for Product Extraction and Titer Determination.

### Example 5: Generation of the Base Strain for TKS Screening

A set of genes for screening for TKS activity was engineered into Saccharomyces cerevisiae in two steps (Table 9). First, constructs were integrated into chromosomal loci to express three genes: a heterologous Zymomonas mobilis PDC gene and two endogenous S. cerevisiae ACS1 and ALD6 genes, all using GAL-regulon promoters. Second, constructs were integrated into chromosomal loci to express AAE and OAC genes from Cannabis sativa (2 and 4 copies, respectively). The resulting strain was capable of producing olivetolic acid in the presence of a TKS enzyme when fed a mixture of sucrose and hexanoic acid, as described in Example 2: Culturing of Yeast. Olivetolic acid was utilized as the reporter for TKS activity, as the tetraketide-CoA intermediate is difficult to measure analytically.

TABLE 9
Representation of the cannabinoid pathway in the engineered
S. cerevisiae strain designed for TKS screening.

| Enzyme | SEQ ID NOs | Copy number and Promoter |
|---|---|---|
| Zm.PDC | Sequence 65 | 1 × pGAL7 |
| Sc.ACS1 | Sequence 66 | 1 × pGAL10 |
| Sc.ALD6 | Sequence 67 | 1 × pGAL1 |
| Cs.AAE | Sequence 6 | 2 × pGAL10 |
| Cs.OAC | Sequence 44 | 2 × pGAL1, 2 × pGAL10 |

To measure the activity of proteins with potential TKS activity in vivo in S. cerevisiae, a landing pad was introduced into a screening strain, which allows for the rapid insertion of TKS variants. The landing pad consists of 500 bp of locus-targeting DNA sequences on either end of the construct to the genomic region upstream and downstream of the yeast locus of choice (Upstream locus and Downstream locus), thereby deleting the locus when the landing pad is integrated into the yeast chromosome as shown in FIG. 11. Internally, the landing pad contains a promoter which can be GAL1, GAL3 or any other promoter of yeast GAL regulon, and a yeast terminator of choice flanking an endonuclease recognition site (F-Cphl). The DNA sequences from the TKS library were used to transform the strain along with a plasmid expressing endonuclease F-Cphl, which cuts the recognition sequence, creating a double strand break at the landing pad, and facilitating homologous recombination of the DNA variants at the site. At least six colonies from each transformation were used to screen for TKS activity, using methods described in Example 2: Culturing of Yeast and Example 3: Analytical Methods for Product Extraction and Titer Determination.

### Example 6: Generation of the Base Strain for OAC Screening

A set of genes for screening for OAC activity was engineered into Saccharomyces cerevisiae in two steps (Table 10). First, constructs were integrated into chromosomal loci to express three genes: a heterologous Zymomonas mobilis PDC gene and two endogenous S. cerevisiae ACS1 and ALD6 genes, all using GAL-regulon promoters. Second, constructs were integrated into chromosomal loci to express AAE and TKS genes from Cannabis sativa (2 copies of each). The resulting strain was capable of producing olivetolic acid in the presence of an OAC enzyme when fed a mixture of sucrose and hexanoic acid, as described in Example 2: Culturing of Yeast.

TABLE 10
Representation of the cannabinoid pathway in the engineered
S. cerevisiae strain designed for OAC screening.

| Enzyme | SEQ ID NOs | Copy number and Promoter |
|---|---|---|
| Zm.PDC | Sequence 65 | 1 × pGAL7 |
| Sc.ACS1 | Sequence 66 | 1 × pGAL10 |
| Sc.ALD6 | Sequence 67 | 1 × pGAL1 |
| Cs.AAE | Sequence 6 | 2 × pGAL10 |
| Cs.TKS | Sequence 26 | 2 × pGAL1 |

To measure the activity of proteins with potential OAC activity in vivo in S. cerevisiae, a landing pad was introduced into a screening strain, which allows for the rapid insertion of OAC variants. The landing pad consists of 500 bp of locus-targeting DNA sequences on either end of the construct to the genomic region upstream and downstream of the yeast locus of choice (Upstream locus and Downstream locus), thereby deleting the locus when the landing pad is integrated into the yeast chromosome as shown in FIG. 11. Internally, the landing pad contains a promoter which can be GAL1, GAL3 or any other promoter of yeast GAL regulon, and a yeast terminator of choice flanking an endonuclease recognition site (F-Cphl). The DNA sequences from the OAC library were used to transform the strain along with a plasmid expressing endonuclease F-Cphl, which cuts the recognition sequence, creating a double strand break at the landing pad, and facilitating homologous recombination of the DNA variants at the site. At least six colonies from each transformation were used to screen for OAC activity, using methods described in Example 2: Culturing of Yeast and Example 3: Analytical Methods for Product Extraction and Titer Determination.

### Example 7: Identification of Novel Proteins with AAE Activity from a Natural Diversity Library

A library of enzymes was generated to identify enzymes capable of catalyzing the formation of hexanoyl-CoA with improved properties over the previously identified AAE enzyme Cs.AAE (SEQ ID NO: 6) from the plant Cannabis sativa. The ligation of a fatty acid to Coenzyme A (CoA) is a ubiquitous reaction in biological systems, where it is catalyzed by adenylate-forming enzymes (Schmelz et al., Curr. Opin. Struc. Biol., 19:6, 2009, 666-71). This bioorganic chemistry has convergently evolved in proteins with vastly different domain architectures, so a homology-based search of sequence databases is insufficient to retrieve the full suite of enzymes with this desired catalytic activity. Instead, we chose to leverage protein annotations in the UniProt Knowledgebase (UniProtKB) to generate a list of candidate sequences for functional characterization.

We began by searching UniProtKB for proteins annotated with the Enzyme Commission (EC) numbers 6.2.1.2, for medium-chain acyl-CoA ligase, or 6.2.1.3, for long-chain acyl-CoA ligase. The database was accessed on Nov. 6, 2019, and a total of 18,245 protein sequences were obtained. Next, these sequences were then algorithmically clustered at a 30% identity cutoff using CD-HIT (http://weizhong-lab.ucsd.edu/cdhit-web-server/cgi-bin/index.cgi?cmd=cd-hit). To exclude aberrant proteins, only clusters containing at least 5 sequences or containing a protein with “Reviewed” status in UniProtKB were considered for functional characterization. To select a subset with minimal redundancy, one representative sequence was selected from each cluster. Ultimately, 128 proteins were codon-optimized for S. cerevisiae and ordered from a DNA-synthesis vendor.

This library of genes was then screened in an engineered S. cerevisiae strain described in Example 4: Generation of the Base Strain for AAE Screening. The immediate product of the AAE is hexanoyl-CoA, but olivetolic acid was used a primary readout for AAE activity, as a functional AAE increases olivetolic acid production; downstream enzymes, TKS, and OAC, were not limiting in this screening strain.

Out of 128 proteins in the Natural Diversity Library screened, 23 of them produced olivetolic acid at 0.21- to 1.27-fold the amount of Cs.AAE (FIG. 2). These 23 proteins (SEQ ID NOS: 1, 2, 3, 4, 5, and 7 through 24) that produced olivetolic acid at least one standard deviation higher than the screening strain were classified as hits. Each of these proteins shares less than 30% sequence similarity with the AAE from Cannabis sativa (SEQ ID NO: 6).

The four proteins displaying the highest AAE activity each share less than 20% sequence similarity with Cs.AAE and achieve higher amounts of olivetolic acid in engineered S. cerevisiae strains. These four proteins come from the bacterial source organisms Pseudonocardia sp. N23 (SEQ ID NO: 1), Pseudomonas sp. (SEQ ID NO: 2), Streptomyces sp.ADI96-02 (SEQ ID NO: 3), and Erythrobacter citreus LAMA 915 (SEQ ID NO: 4). There are several potential reasons for why certain heterologous AAE proteins may achieve better microbial production of cannabinoids, compared to the AAE from Cannabis sativa, such as improved folding, stability, K_{M}, K_{cat}, pH preference, cofactor requirement (e.g. Mg²⁺), and substrate specificity. A strength of the in vivo screening platform utilized here is to identify optimal enzymes in a context that resembles microbial production of cannabinoids at manufacturing scale.

### Example 8: Identification of Novel Proteins with TKS Activity from a Natural Diversity Library

A library of candidate protein sequences was assembled using two different approaches to identify enzymes having tetraketide synthase (TKS) activity. The first approach relied on homology searching using a TKS (SEQ ID NO: 26) known to participate in olivetolic acid biosynthesis from Cannabis sativa as a query sequence. The query sequence was used to perform three iterations of position specific iterative basic local alignment search tool (PSI-BLAST, Altschul et al, Nuc. Acid Research, 25:17, 1997, 3389-3402) against a pre-clustered protein database (UniRef90, Baris et al, Bioinformatics, 31:6, 2015, 926-32). The resultant position specific scoring matrix (PSSM) was used to query all known protein sequences stored by the National Center for Biotechnology Information (NCBI-nr/RefSeq non-redundant) resulting in several thousand amino acid sequences. Sequences were clustered based on pairwise amino acid similarity using CD-HIT, and candidate sequences were chosen manually from the resultant clusters to add to the library.

The second approach used SciFinder (CAS, Limin et al., Bioinformatics, 28:23, 2012, 3150-52) to locate hundreds of literature references related to the biosynthesis of alkylated resorcylic acid derivatives. References related to short chain derivatives resembling olivetolic acid (a pentyl-derivative) were closely read for specific mention of biosynthetic genes. Candidate TKS genes were added to the library from the organisms located from the references. Combined, these two approaches yielded 90 candidate protein sequences. The protein sequences were codon-optimized for S. cerevisiae and ordered from a DNA-synthesis vendor.

This library of genes was then screened in an engineered S. cerevisiae strain described in Example 5: Generation of the Base Strain for TKS Screening. The immediate product of the TKS is tetraketide-CoA, but olivetolic acid was used a primary readout for TKS activity, as a functional TKS is strictly necessary for olivetolic acid production; upstream and downstream enzymes (AAE and OAC, respectively) were not limiting in this screening strain.

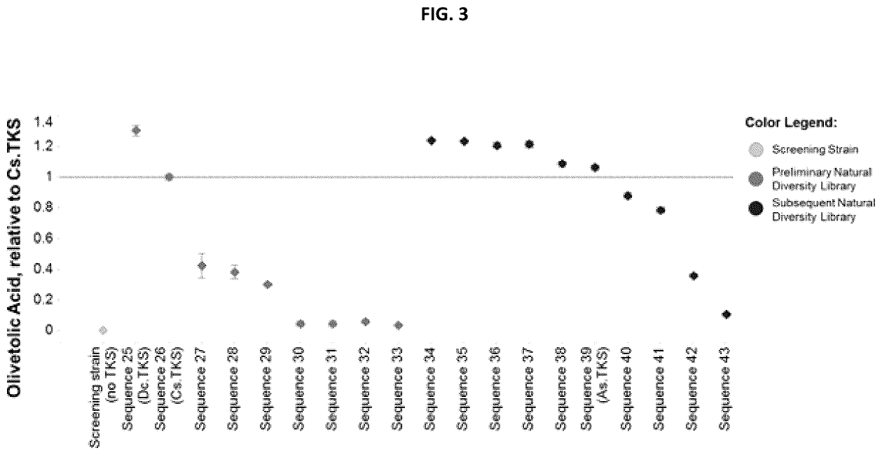

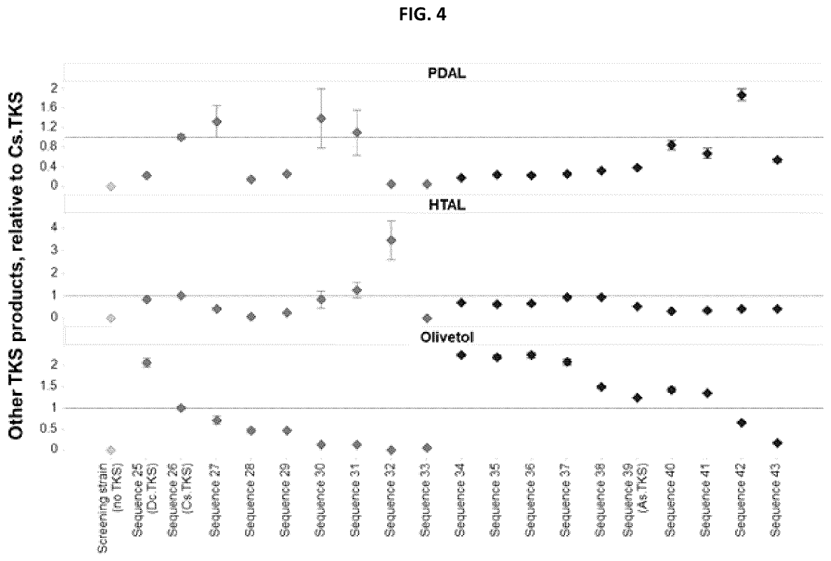

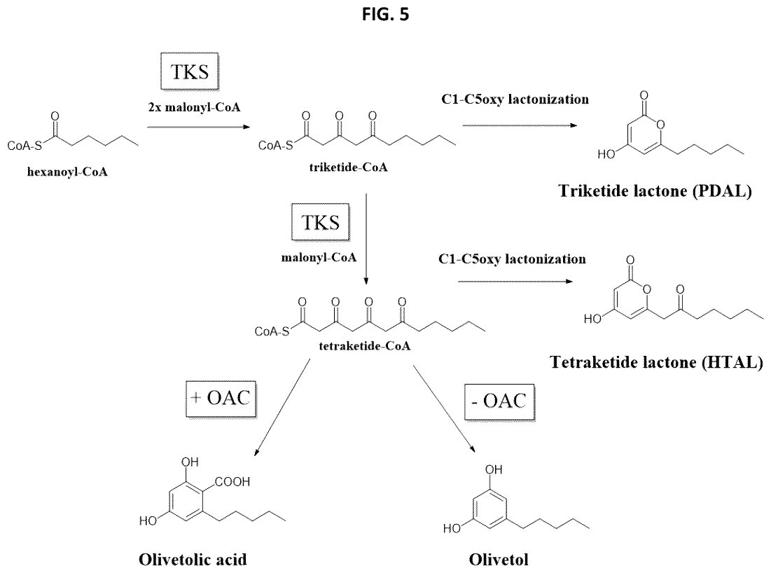

After screening, several hits were observed that produce olivetolic acid (FIG. 3). Notably, many of the hits also produced HTAL, PDAL, and olivetol in addition to olivetolic acid (FIG. 4). These molecules can be formed as part of TKS catalysis, and when present, are also indicative of an active TKS enzyme as shown in the TKS reaction mechanism in FIG. 5. Overall, of the 90 genes screened, the preliminary library resulted in 8 novel TKS proteins (SEQ ID NOS: 25, 27, 28, 29, 30, 31, 32, 33) that produced between 0.07-fold and 1.30-fold the amount of olivetolic acid compared to the TKS from Cannabis sativa (Cs.TKS, SEQ ID NO: 26). Each of these novel TKS proteins shares less than 70% similarity with Cs.TKS.

One protein (SEQ ID NO: 25) from this initial TKS Natural Diversity Library achieved particularly high olivetolic acid production compared to Cs. TKS in engineered S. cerevisiae strains. This most active TKS protein comes from source organism Dendrobium catenatum, a species of lithophytic orchid. Motivated by the surprisingly high TKS activity of this enzyme (Dc.TKS, SEQ ID NO: 25), we performed an additional homology search to identify more protein sequences from this clade of natural diversity. The BLASTp algorithm (https://blast.ncbi.nlm.nih.gov/Blast.cgi) was used to gather all proteins from NCBI-nr database with >70% amino acid identity to Dc.TKS. The resulting 38 sequences were clustered based on sequence identity using CD-HIT with a 93% identity threshold, giving a final list of 14 proteins. The protein sequences were codon-optimized for S. cerevisiae and ordered from a DNA-synthesis vendor.

Screening the subsequent natural diversity library yielded an additional 10 proteins (SEQ ID NOS: 34 through 43) possessing TKS activity, including an additional 6 proteins (SEQ ID NOS: 34, 35, 36, 37, 38, 39) that surpass the activity of the TKS from Cannabis sativa (FIG. 3). The source organisms of these novel TKS proteins are a variety of orchid species. Several of these novel TKS proteins also produce higher amounts of olivetol and lower amounts of PDAL, compared to Cs.TKS (FIG. 4). In particular, a TKS from Apostasia shenzhenica (As.TKS, SEQ ID NO: 39) produces higher olivetolic acid while not producing much higher olivetol. The favorable product profile of As. TKS and the high activity of Dc.TKS (SEQ ID NO: 25), as demonstrated in this in vivo characterization, are thus advantageous for the microbial production of cannabinoids.

### Example 9: Identification of Proteins with Improved OAC Activity from Site Saturation Mutagenesis followed by Combinatorial Mutagenesis

The OAC enzyme catalyzes the cyclization of tetraketide-CoA into olivetolic acid (Gagne S L et al. PNAS 109:31, 2012, 12811-12816). In this example, site-saturation mutagenesis was used to improve the activity of OAC from Cannabis sativa (SEQ ID NO: 44). Each amino acid residue was mutated using the degenerate codon NNT, where “N” indicates any of the four nucleotides. The degenerate codon NNT can encode 15 different amino acids (A, C, D, F, G, H, I, L, N, P, R, S, T, V, and Y). Each library for a given amino acid residue was generated by PCR and transformed into the screening strain described in Example 6: Generation of the Base Strain for OAC Screening.

In primary screening (termed Tier 1), 26 colonies per library were tested using the conditions described in Example 2: Culturing of Yeast and the high-throughput assay described in Example 3: Analytical Methods for Product Extraction and Titer Determination. In a secondary screen (termed Tier 2), transformed strains harboring OAC enzyme mutants of interest were re-tested in higher replication (n≥6) to determine if the improved activity was significant. A mutation was considered to improve OAC activity if the median amount of olivetolic acid produced by the mutant was at least one standard deviation above the median amount of olivetolic acid produced by the original Cs.OAC protein.

Nine unique point mutations that improved OAC activity were identified: K49R, T47R, V28L, E14S, K12S, L91, L92Y, A2S, and F23L. The OAC activity of each of these mutants is provided in Table 11. These individual mutants resulted in up to 1.34-fold the production olivetolic acid, compared to the original Cs.OAC (SEQ ID NO: 44). An additional three point mutations that have a neutral effect on OAC activity are also provided in Table 11: Q48R, S87H, and F88Y. These combined twelve point mutations were then used to generate a full factorial combinatorial library, with the intent to obtain mutant proteins with further improvements in OAC activity.

The full factorial combinatorial library was generated by PCR and transformed into the strain described in Example 6: Generation of the Base Strain for OAC Screening. A total of 3579 colonies from this pooled library transformation were tested in Tier-1 screening. Subsequently, 360 of the colonies were tested in higher replication in Tier-2 screening, as described above, and also sequenced to determine the DNA coding sequence of the protein combinatorial mutant.

In total, 167 unique protein sequences, each containing at least six amino acid point mutations, were found to possess improved OAC activity. These combinatorial mutants resulted in between 1.55-fold and 2.31-fold the production of olivetolic acid, compared to Cs.OAC. Each of these improved OAC proteins shares less than 95% similarity to Cs.OAC (SEQ ID NO: 44). The sequences and OAC activity of these proteins are summarized in Table 11.

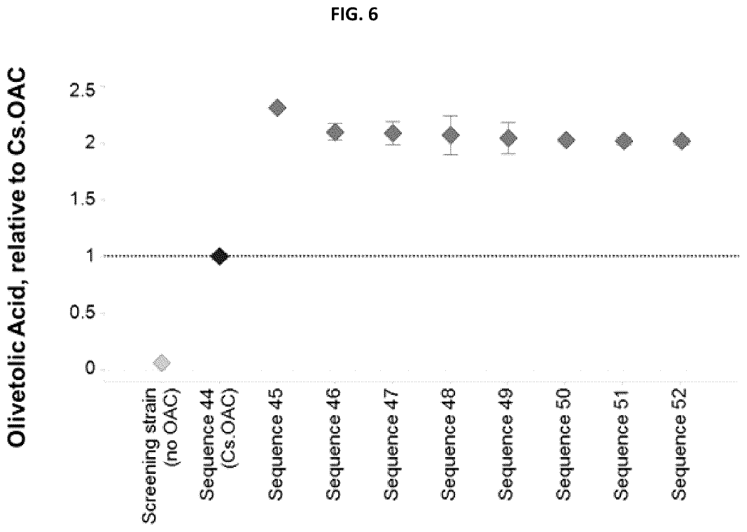

In particular, 8 unique protein sequences, each containing at least eight amino acid point mutations, were found to possess more than double the OAC activity of Cs.OAC (FIG. 6). These combinatorial mutants (SEQ ID NOs: 45, 46, 47, 48, 49, 50, 51, 52) achieve up to 2.31-fold the production of olivetolic acid, compared to Cs.OAC. The top 8 improved OAC proteins each share less than 93% similarity to Cs.OAC (SEQ ID NO: 44). The identification of these improved enzymes will aid in the production of cannabinoids at high purity and lower cost.

TABLE 11
Cs.OAC sequence and activity data

| | Relative | | |
|---|---|---|---|
| | Olivetolic | | #AA |
| Seq ID | Acid titer | Mutations | diff |
| SEQ ID NO 44 | 1.00 | Parent Enzyme Cs.OAC | 0 |
| single v1 | 0.92 | Q48R | 1 |
| single v2 | 0.94 | S87H | 1 |
| single v3 | 1.01 | F88Y | 1 |
| single v4 | 1.06 | K49R | 1 |
| single v5 | 1.06 | T47R | 1 |
| single v6 | 1.07 | V28L | 1 |
| single v7 | 1.16 | E14S | 1 |
| single v8 | 1.20 | K12S | 1 |
| single v9 | 1.21 | L9I | 1 |
| single v10 | 1.21 | L92Y | 1 |
| single v11 | 1.23 | A2S | 1 |
| single v12 | 1.34 | F23L | 1 |
| combi v1 = | 2.31 | L9I, K12S, E14S, V28L, Q48R, K49R, S87H, F88Y | 8 |
| SEQ ID NO 45 | | | |
| combi v2 = | 2.10 | A2S, L9I, K12S, F23L, T47R, K49R, S87H, F88Y | 8 |
| SEQ ID NO 46 | | | |
| combi v3 = | 2.09 | A2S, L9I, K12S, F23L, Q48R, K49R, S87H, F88Y | 8 |
| SEQ ID NO 47 | | | |
| combi v4 = | 2.07 | A2S, L9I, E14S, F23L, Q48R, K49R, S87H, F88Y | 8 |
| SEQ ID NO 48 | | | |
| combi v5 = | 2.06 | A2S, L9I, E14S, F23L, V28L, Q48R, K49R, F88Y | 8 |
| SEQ ID NO 49 | | | |
| combi v6 = | 2.02 | L9I, K12S, E14S, F23L, Q48R, K49R, S87H, F88Y | 8 |
| SEQ ID NO 50 | | | |
| combi v7 = | 2.02 | A2S, L9I, K12S, E14S, Q48R, K49R, S87H, F88Y | 8 |
| SEQ ID NO 51 | | | |
| combi v8 = | 2.02 | A2S, L9I, E14S, F23L, T47R, Q48R, K49R, F88Y | 8 |
| SEQ ID NO 52 | | | |
| combi v9 | 1.76 | A2S, L9I, K12S, E14S, F23L, V28L, Q48R, K49R, S87H, | 10 |
| | | F88Y | |
| combi v10 | 1.94 | A2S, L9I, K12S, E14S, V28L, Q48R, K49R, S87H, F88Y | 9 |
| combi v11 | 1.93 | A2S, L9I, E14S, F23L, V28L, Q48R, K49R, S87H, F88Y | 9 |
| combi v12 | 1.88 | A2S, L9I, K12S, V28L, T47R, Q48R, K49R, S87H, F88Y | 9 |
| combi v13 | 1.88 | A2S, L9I, E14S, F23L, T47R, Q48R, K49R, S87H, F88Y | 9 |
| combi v14 | 1.88 | A2S, L9I, K12S, E14S, F23L, V28L, Q48R, K49R, F88Y | 9 |
| combi v15 | 1.86 | A2S, L9I, K12S, F23L, V28L, Q48R, K49R, S87H, F88Y | 9 |
| combi v16 | 1.83 | A2S, L9I, E14S, V28L, T47R, Q48R, K49R, S87H, F88Y | 9 |
| combi v17 | 1.75 | A2S, L9I, K12S, F23L, T47R, Q48R, K49R, S87H, F88Y | 9 |
| combi v18 | 1.65 | A2S, L9I, E14S, F23L, V28L, T47R, K49R, S87H, F88Y | 9 |
| combi v19 | 1.95 | A2S, L9I, E14S, V28L, T47R, Q48R, K49R, F88Y | 8 |
| combi v20 | 1.94 | A2S, L9I, E14S, F23L, T47R, K49R, S87H, F88Y | 8 |
| combi v21 | 1.94 | A2S, L9I, F23L, V28L, Q48R, K49R, S87H, F88Y | 8 |
| combi v22 | 1.92 | A2S, L9I, K12S, E14S, T47R, K49R, S87H, F88Y | 8 |
| combi v23 | 1.92 | A2S, L9I, E14S, F23L, V28L, K49R, S87H, F88Y | 8 |
| combi v24 | 1.91 | A2S, L9I, E14S, F23L, V28L, Q48R, K49R, S87H | 8 |
| combi v25 | 1.91 | A2S, L9I, K12S, E14S, V28L, K49R, S87H, F88Y | 8 |
| combi v26 | 1.90 | L9I, E14S, F23L, T47R, Q48R, K49R, S87H, F88Y | 8 |
| combi v27 | 1.90 | A2S, L9I, F23L, T47R, Q48R, K49R, S87H, F88Y | 8 |
| combi v28 | 1.90 | A2S, L9I, K12S, E14S, F23L, V28L, K49R, F88Y | 8 |
| combi v29 | 1.90 | A2S, L9I, E14S, V28L, T47R, K49R, S87H, F88Y | 8 |
| combi v30 | 1.88 | A2S, L9I, K12S, F23L, V28L, Q48R, K49R, F88Y | 8 |
| combi v31 | 1.84 | A2S, L9I, E14S, F23L, V28L, T47R, K49R, F88Y | 8 |
| combi v32 | 1.84 | A2S, L9I, V28L, T47R, Q48R, K49R, S87H, F88Y | 8 |
| combi v33 | 1.83 | A2S, L9I, E14S, F23L, T47R, Q48R, K49R, S87H | 8 |
| combi v34 | 1.82 | L9I, K12S, F23L, V28L, Q48R, K49R, S87H, F88Y | 8 |
| combi v35 | 1.82 | A2S, L9I, K12S, F23L, V28L, K49R, S87H, F88Y | 8 |
| combi v36 | 1.81 | A2S, L9I, K12S, V28L, T47R, Q48R, S87H, F88Y | 8 |
| combi v37 | 1.81 | L9I, K12S, E14S, F23L, V28L, Q48R, K49R, F88Y | 8 |
| combi v38 | 1.79 | A2S, L9I, K12S, E14S, F23L, T47R, K49R, F88Y | 8 |
| combi v39 | 1.78 | A2S, L9I, K12S, F23L, V28L, Q48R, K49R, S87H | 8 |
| combi v40 | 1.75 | A2S, L9I, F23L, V28L, T47R, Q48R, K49R, F88Y | 8 |
| combi v41 | 1.75 | L9I, K12S, F23L, T47R, Q48R, K49R, S87H, F88Y | 8 |
| combi v42 | 1.73 | A2S, L9I, K12S, F23L, V28L, Q48R, S87H, F88Y | 8 |
| combi v43 | 1.69 | A2S, L9I, F23L, V28L, T47R, Q48R, S87H, F88Y | 8 |
| combi v44 | 1.67 | L9I, E14S, F23L, V28L, T47R, Q48R, K49R, F88Y | 8 |
| combi v45 | 2.16 | A2S, L9I, E14S, F23L, Q48R, K49R, F88Y | 7 |
| combi v46 | 2.11 | A2S, L9I, K12S, E14S, V28L, K49R, F88Y | 7 |
| combi v47 | 2.05 | A2S, L9I, K12S, T47R, K49R, S87H, F88Y | 7 |
| combi v48 | 2.05 | A2S, L9I, F23L, V28L, Q48R, K49R, F88Y | 7 |
| combi v49 | 2.05 | A2S, L9I, E14S, F23L, Q48R, K49R, S87H | 7 |
| combi v50 | 2.05 | A2S, L9I, E14S, V28L, Q48R, K49R, F88Y | 7 |
| combi v51 | 2.05 | A2S, L9I, K12S, F23L, K49R, S87H, F88Y | 7 |
| combi v52 | 2.04 | L9I, K12S, F23L, Q48R, K49R, S87H, F88Y | 7 |
| combi v53 | 2.02 | A2S, L9I, F23L, T47R, Q48R, K49R, F88Y | 7 |
| combi v54 | 2.02 | A2S, L9I, E14S, F23L, V28L, K49R, F88Y | 7 |
| combi v55 | 2.02 | A2S, L9I, E14S, F23L, T47R, Q48R, K49R | 7 |
| combi v56 | 2.02 | A2S, L9I, E14S, F23L, V28L, Q48R, K49R | 7 |
| combi v57 | 2.01 | L9I, K12S, E14S, F23L, K49R, S87H, F88Y | 7 |
| combi v58 | 2.00 | L9I, K12S, E14S, Q48R, K49R, S87H, F88Y | 7 |
| combi v59 | 2.00 | L9I, K12S, T47R, Q48R, K49R, S87H, F88Y | 7 |
| combi v60 | 1.98 | A2S, L9I, K12S, F23L, Q48R, K49R, S87H | 7 |
| combi v61 | 1.98 | L9I, E14S, F23L, V28L, Q48R, K49R, F88Y | 7 |
| combi v62 | 1.97 | L9I, K12S, V28L, Q48R, K49R, S87H, F88Y | 7 |
| combi v63 | 1.97 | A2S, L9I, E14S, T47R, Q48R, K49R, F88Y | 7 |
| combi v64 | 1.96 | A2S, L9I, F23L, T47R, Q48R, S87H, F88Y | 7 |
| combi v65 | 1.95 | A2S, L9I, K12S, E14S, T47R, K49R, F88Y | 7 |
| combi v66 | 1.94 | A2S, L9I, E14S, F23L, V28L, K49R, S87H | 7 |
| combi v67 | 1.94 | A2S, L9I, E14S, V28L, Q48R, K49R, S87H | 7 |
| combi v68 | 1.94 | A2S, L9I, K12S, F23L, Q48R, S87H, F88Y | 7 |
| combi v69 | 1.93 | A2S, L9I, E14S, F23L, V28L, T47R, F88Y | 7 |
| combi v70 | 1.93 | A2S, L9I, E14S, F23L, T47R, S87H, F88Y | 7 |
| combi v71 | 1.92 | A2S, L9I, F23L, V28L, Q48R, K49R, S87H | 7 |
| combi v72 | 1.92 | A2S, L9I, F23L, T47R, K49R, S87H, F88Y | 7 |
| combi v73 | 1.92 | A2S, L9I, V28L, T47R, K49R, S87H, F88Y | 7 |
| combi v74 | 1.92 | A2S, L9I, E14S, F23L, Q48R, S87H, F88Y | 7 |
| combi v75 | 1.91 | L9I, K12S, F23L, V28L, Q48R, K49R, F88Y | 7 |
| combi v76 | 1.91 | L9I, K12S, E14S, F23L, Q48R, K49R, S87H | 7 |
| combi v77 | 1.91 | A2S, L9I, K12S, E14S, T47R, Q48R, F88Y | 7 |
| combi v78 | 1.90 | A2S, K12S, V28L, Q48R, K49R, S87H, F88Y | 7 |
| combi v79 | 1.89 | A2S, L9I, K12S, V28L, T47R, S87H, F88Y | 7 |
| combi v80 | 1.89 | A2S, L9I, K12S, F23L, V28L, K49R, F88Y | 7 |
| combi v81 | 1.89 | A2S, L9I, F23L, T47R, Q48R, K49R, S87H | 7 |
| combi v82 | 1.87 | L9I, E14S, F23L, T47R, K49R, S87H, F88Y | 7 |
| combi v83 | 1.87 | A2S, L9I, E14S, T47R, Q48R, K49R, S87H | 7 |
| combi v84 | 1.87 | L9I, E14S, V28L, Q48R, K49R, S87H, F88Y | 7 |
| combi v85 | 1.86 | A2S, L9I, F23L, V28L, T47R, K49R, F88Y | 7 |
| combi v86 | 1.86 | L9I, E14S, F23L, V28L, Q48R, K49R, S87H | 7 |
| combi v87 | 1.84 | L9I, K12S, E14S, F23L, V28L, K49R, F88Y | 7 |
| combi v88 | 1.84 | A2S, L9I, E14S, F23L, T47R, Q48R, S87H | 7 |
| combi v89 | 1.83 | L9I, F23L, V28L, Q48R, K49R, S87H, F88Y | 7 |
| combi v90 | 1.83 | A2S, L9I, F23L, V28L, T47R, Q48R, F88Y | 7 |
| combi v91 | 1.82 | L9I, K12S, F23L, V28L, K49R, S87H, F88Y | 7 |
| combi v92 | 1.81 | L9I, K12S, E14S, V28L, Q48R, S87H, F88Y | 7 |
| combi v93 | 1.80 | L9I, E14S, V28L, T47R, Q48R, S87H, F88Y | 7 |
| combi v94 | 1.79 | A2S, L9I, V28L, T47R, Q48R, S87H, F88Y | 7 |
| combi v95 | 1.78 | A2S, L9I, E14S, V28L, T47R, Q48R, S87H | 7 |
| combi v96 | 1.76 | A2S, L9I, K12S, E14S, F23L, V28L, K49R | 7 |
| combi v97 | 1.74 | L9I, E14S, F23L, V28L, T47R, Q48R, F88Y | 7 |
| combi v98 | 1.60 | A2S, L9I, F23L, K49R, S87H, F88Y, L92Y | 7 |
| combi v99 | 2.11 | A2S, L9I, F23L, Q48R, K49R, S87H | 6 |
| combi v100 | 2.11 | A2S, L9I, K12S, F23L, K49R, S87H | 6 |
| combi v101 | 2.09 | A2S, L9I, F23L, K49R, S87H, F88Y | 6 |
| combi v102 | 2.08 | L9I, E14S, F23L, T47R, Q48R, F88Y | 6 |
| combi v103 | 2.05 | L9I, K12S, E14S, V28L, K49R, F88Y | 6 |
| combi v104 | 2.05 | A2S, L9I, K12S, F23L, Q48R, K49R | 6 |
| combi v105 | 2.05 | A2S, L9I, K12S, F23L, Q48R, F88Y | 6 |
| combi v106 | 2.04 | L9I, K12S, F23L, K49R, S87H, F88Y | 6 |
| combi v107 | 2.04 | A2S, L9I, E14S, F23L, K49R, S87H | 6 |
| combi v108 | 2.03 | A2S, L9I, K12S, V28L, K49R, F88Y | 6 |
| combi v109 | 2.03 | A2S, L9I, E14S, T47R, K49R, F88Y | 6 |
| combi v110 | 2.03 | A2S, L9I, F23L, V28L, K49R, F88Y | 6 |
| combi v111 | 2.03 | A2S, L9I, E14S, F23L, T47R, F88Y | 6 |
| combi v112 | 2.01 | L9I, K12S, V28L, K49R, S87H, F88Y | 6 |
| combi v113 | 2.01 | A2S, L9I, F23L, V28L, Q48R, K49R | 6 |
| combi v114 | 2.01 | A2S, L9I, E14S, V28L, K49R, F88Y | 6 |
| combi v115 | 2.01 | A2S, L9I, K12S, Q48R, K49R, F88Y | 6 |
| combi v116 | 2.00 | A2S, L9I, K12S, E14S, T47R, K49R | 6 |
| combi v117 | 2.00 | A2S, L9I, E14S, F23L, Q48R, F88Y | 6 |
| combi v118 | 2.00 | A2S, L9I, K12S, E14S, K49R, S87H | 6 |
| combi v119 | 1.99 | L9I, K12S, E14S, T47R, K49R, F88Y | 6 |
| combi v120 | 1.99 | A2S, L9I, V28L, Q48R, K49R, F88Y | 6 |
| combi v121 | 1.99 | A2S, L9I, E14S, Q48R, K49R, F88Y | 6 |
| combi v122 | 1.98 | A2S, L9I, K12S, Q48R, K49R, S87H | 6 |
| combi v123 | 1.98 | A2S, L9I, K12S, E14S, T47R, F88Y | 6 |
| combi v124 | 1.98 | A2S, L9I, F23L, Q48R, S87H, F88Y | 6 |
| combi v125 | 1.96 | A2S, L9I, K12S, T47R, K49R, S87H | 6 |
| combi v126 | 1.96 | L9I, K12S, E14S, F23L, Q48R, K49R | 6 |
| combi v127 | 1.95 | A2S, L9I, E14S, F23L, T47R, K49R | 6 |
| combi v128 | 1.95 | A2S, L9I, F23L, T47R, Q48R, K49R | 6 |
| combi v129 | 1.94 | L9I, K12S, F23L, V28L, K49R, F88Y | 6 |
| combi v130 | 1.93 | A2S, L9I, E14S, F23L, T47R, Q48R | 6 |
| combi v131 | 1.93 | A2S, L9I, K12S, Q48R, S87H, F88Y | 6 |
| combi v132 | 1.92 | L9I, F23L, V28L, Q48R, K49R, F88Y | 6 |
| combi v133 | 1.91 | L9I, F23L, T47R, Q48R, K49R, F88Y | 6 |
| combi v134 | 1.91 | L9I, F23L, V28L, K49R, S87H, F88Y | 6 |
| combi v135 | 1.91 | A2S, L9I, E14S, F23L, V28L, Q48R | 6 |
| combi v136 | 1.91 | A2S, L9I, V28L, T47R, Q48R, F88Y | 6 |
| combi v137 | 1.90 | A2S, L9I, F23L, V28L, K49R, S87H | 6 |
| combi v138 | 1.90 | A2S, L9I, V28L, T47R, K49R, S87H | 6 |
| combi v139 | 1.89 | L9I, E14S, F23L, V28L, Q48R, S87H | 6 |
| combi v140 | 1.88 | A2S, L9I, Q48R, K49R, S87H, F88Y | 6 |
| combi v141 | 1.88 | L9I, E14S, F23L, T47R, K49R, S87H | 6 |
| combi v142 | 1.88 | A2S, L9I, K12S, E14S, V28L, F88Y | 6 |
| combi v143 | 1.88 | A2S, L9I, E14S, F23L, Q48R, S87H | 6 |
| combi v144 | 1.87 | A2S, E14S, V28L, T47R, K49R, F88Y | 6 |
| combi v145 | 1.87 | L9I, K12S, F23L, Q48R, S87H, F88Y | 6 |
| combi v146 | 1.86 | L9I, E14S, V28L, T47R, Q48R, F88Y | 6 |
| combi v147 | 1.86 | A2S, L9I, K12S, F23L, Q48R, S87H | 6 |
| combi v148 | 1.83 | A2S, L9I, E14S, T47R, K49R, S87H | 6 |
| combi v149 | 1.83 | A2S, L9I, E14S, Q48R, K49R, S87H | 6 |
| combi v150 | 1.82 | A2S, L9I, F23L, V28L, T47R, F88Y | 6 |
| combi v151 | 1.82 | A2S, L9I, V28L, T47R, Q48R, S87H | 6 |
| combi v152 | 1.82 | A2S, L9I, K12S, F23L, T47R, F88Y | 6 |
| combi v153 | 1.81 | A2S, L9I, K12S, F23L, V28L, F88Y | 6 |
| combi v154 | 1.80 | L9I, E14S, F23L, V28L, K49R, S87H | 6 |
| combi v155 | 1.80 | A2S, K12S, E14S, V28L, K49R, F88Y | 6 |
| combi v156 | 1.79 | A2S, E14S, F23L, Q48R, K49R, S87H | 6 |
| combi v157 | 1.79 | A2S, E14S, F23L, V28L, K49R, F88Y | 6 |
| combi v158 | 1.78 | L9I, F23L, V28L, Q48R, K49R, S87H | 6 |
| combi v159 | 1.78 | A2S, L9I, F23L, V28L, Q48R, S87H | 6 |
| combi v160 | 1.77 | A2S, L9I, F23L, V28L, S87H, F88Y | 6 |
| combi v161 | 1.71 | A2S, E14S, F23L, V28L, K49R, S87H | 6 |
| combi v162 | 1.71 | A2S, L9I, E14S, V28L, S87H, F88Y | 6 |
| combi v163 | 1.68 | L9I, K12S, F23L, V28L, S87H, F88Y | 6 |
| combi v164 | 1.67 | L9I, E14S, F23L, K49R, F88Y, L92Y | 6 |
| combi v165 | 1.59 | L9I, F23L, V28L, T47R, K49R, F88Y | 6 |
| combi v166 | 1.56 | A2S, L9I, T47R, Q48R, F88Y, L92Y | 6 |
| combi v167 | 1.55 | A2S, F23L, Q48R, K49R, F88Y, L92Y | 6 |

### Example 10: Generation of the Base Strain for CBGaS Screening

A set of genes for screening for CBGaS activity was engineered into Saccharomyces cerevisiae (Table 12). This strain contains the following chromosomally integrated mevalonate pathway genes from S. cerevisiae: acetyl-CoA thiolase (ERG10), HMG-COA synthase (ERG13), HMG-COA reductase truncated to alleviate feedback inhibition (HMGR-t), mevalonate kinase (ERG12), phosphomevalonate kinase (ERG8), mevalonate pyrophosphate decarboxylase (MVD1), and IPP:DMAPP isomerase (IDI1). In addition, the strain contained copies of four heterologous enzymes involved in the cannabinoid biosynthetic pathway (FIG. 1): the acyl-activating enzyme (AAE), tetraketide synthase (TKS), and olivetolic acid cyclase (OAC) from Cannabis sativa, as well as geranyl pyrophosphate (GPP) synthase from Streptomyces aculeolatus, all under the control of GAL regulated promoters. To increase flux to cytosolic acetyl-CoA, PDC from Zymomonas mobilis, and overexpression of S. cerevisiae ALD6 and ACS1 were included in the engineering. FIG. 1 shows a depiction of the biosynthetic pathway to cannabigerolic acid (CBGA) utilized in the screening strain, with enzyme screening occurring at the PT node.

TABLE 12
Representation of the cannabinoid pathway in the engineered
S. cerevisiae strain designed for CBGaS screening.

| Enzyme | SEQ ID NOs | Copy number and Promoter |
|---|---|---|
| Zm.PDC | Sequence 65 | 1 × pGAL7 |
| Sc.ACS1 | Sequence 66 | 1 × pGAL10 |
| Sc.ALD6 | Sequence 67 | 1 × pGAL1 |
| Cs.AAE | Sequence 6 | 2 × pGAL10 |
| Cs.TKS | Sequence 26 | 2 × pGAL10 |
| Cs.OAC | Sequence 44 | 4 × pGAL1 |
| Sc.ERG10 | Sequence 68 | 1 × pGAL2 |
| Sc.ERG13 | Sequence 69 | 1 × pGAL1 |
| Sc.HMGR-t | Sequence 70 | 1 × pGAL10 |
| Sc.ERG12 | Sequence 71 | 1 × pGAL2 |
| Sc.ERG8 | Sequence 72 | 1 × pGAL1 |
| Sc.MVD1 | Sequence 73 | 1 × pGAL10 |
| Sc.IDI1 | Sequence 74 | 1 × pGAL7 |
| Sa.GPPS | Sequence 75 | 1 × pGAL10 |

In order to screen the library of candidate genes for CBGaS activity, a landing pad approach was utilized (FIG. 11). An intergenic region in the screening strain was altered to contain an F-Cphl endonuclease recognition site, which was flanked by a GAL-regulon promoter and a terminator, both from yeast as described in U.S. Pat. No. 7,919,605B1, which is incorporated herein by reference. This site allowed the candidate genes to be integrated into the genome by co-transformation of the endonuclease F-Cphl alongside donor DNA containing the desired DNA sequence to be screened, flanked by 40 base pair homology regions to the promoter and terminator. At least six colonies from each transformation were used to screen for CBGaS activity, using methods described in Example 2: Culturing of Yeast and Example 3: Analytical Methods for Product Extraction and Titer Determination.

### Example 11: Identification of Novel Proteins with CBGaS Activity from a Natural Diversity Library

A previously identified CBGA synthase (CBGaS) enzyme Cs.PT4 (SEQ ID NO: 53) from the plant Cannabis sativa belongs to the UbiA protein family. Members of the UbiA family occur in all three domains of life and are known to catalyze diverse prenylation reactions of aromatic substrates (Li, W., Trends Bioche. Sci., 41:4, 2016, 356-70). However, proteins of the UbiA family that catalyze the formation of CBGA are extremely rare. Here, we pursued two alternate hypotheses novel proteins with improved CBGaS activity: (1) Plant UbiA proteins related to Cs.PT4 also possess CBGaS catalytic activity, and (2) UbiA proteins possessing CBGaS activity exist in organisms that produce chemicals structurally similar to CBGA.

To pursue the first hypothesis, Cs.PT4 was used to search for homologous proteins in the three following sequence databases: NCBI RefSeq non-redundant proteins UniProt Knowledgebase (UniProtKB), and JGI Phytozome v12.1 Proteomes (https://phytozome.jgi.doe.gov/pz/portal.html). These databases were accessed on Feb. 20, 2019. An E-value threshold of 1e-20 for the BLASTp algorithm was used to gather sequences that range from about 23% to about 48% identity to Cs.PT4. The combined results from the three databases consisted of 1059 protein sequences. These sequences were then algorithmically clustered at a 70% identity cutoff using CD-HIT (http://weizhong-lab.ucsd.edu/cdhit-web-server/cgi-bin/index.cgi?cmd=cd-hit). To minimize redundancy in the sequences to be functionally characterized, one representative was selected from each cluster. Ultimately, 172 proteins were codon-optimized for S. cerevisiae and ordered from an appropriate gene-synthesis vendor. This set of sequences is termed the homology library.

The plant UbiA sequences typically contain an N-terminal chloroplast transit peptide (cTP), which is known to impair the expression of such proteins in microbial hosts such as S. cerevisiae. For example, removal of the cTP from the Cannabis sativa protein Cs.PT4 (SEQ ID NO: 53) to generate the truncated protein Cs.PT4-T (SEQ ID NO: 54) significantly improves the CBGaS activity in engineered S. cerevisiae strains. Accordingly, for each of the plant UbiA proteins in the natural diversity library, a set of primers was designed to truncate the computationally predicted cTP (http://www.cbs.dtu.dk/services/ChloroP/). This in effect added an additional 172 proteins to the homology library.

To pursue the second hypothesis, SciFinder (https://scifinder-n.cas.org/) was used to search the academic literature for reports of chemicals containing a prenylated resorcylic acid substructure, resembling that of CBGA. Papers related to natural products bearing this chemical substructure were closely read for mention of the producing organism. Suspected UbiA-type prenyltransferase proteins were then identified from these target species in UniProtKB. Ultimately, 15 proteins were codon-optimized for S. cerevisiae and ordered from an appropriate gene-synthesis vendor. This set of sequences is termed the target species library.

Each member of the homology and target-species libraries was transformed individually into the strain described in Example 10: Generation of the Base Strain for CBGaS Screening. The resulting yeast strains were screened for the ability to produce CBGA using methods described in Example 2: Culturing of Yeast and Example 3: Analytical Methods for Product Extraction and Titer Determination.

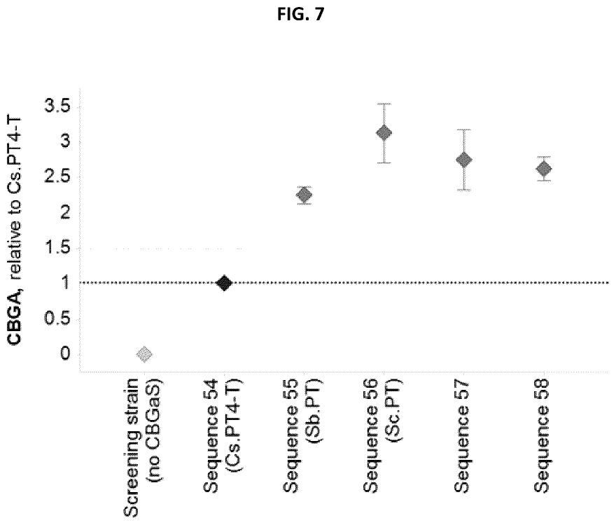

The majority of the proteins in these natural diversity libraries were devoid of any CBGaS activity. However, one protein from the target species library resulted in CBGA production that was 2.24-fold the level of Cs.PT4-T (SEQ ID NO: 54) (FIG. 7). This novel CBGaS enzyme is from the fungal source organism Stachybotrys bisbyi and is hereafter referred to as Sb.PT (SEQ ID NO: 55). The novel CBGaS protein Sb.PT shares less than 20% sequence similarity to Cs.PT4-T.

Motivated by the surprisingly high CBGaS activity of Sb.PT, we performed an additional homology search to identify more protein sequences from this clade of natural diversity. The BLASTp algorithm (https://blast.ncbi.nlm.nih.gov/Blast.cgi) was used to gather all proteins from NCBI-nr and UniProtKB databases with >50% amino acid identity to Sb.PT. The resulting 11 proteins were codon-optimized for S. cerevisiae, ordered from a DNA-synthesis vendor, and then screened for CBGaS activity.

Screening the subsequent natural diversity library yielded an additional 3 proteins possessing CBGaS activity (FIG. 7). Two of the novel CBGaS enzymes are from the fungal source organism Stachybotrys chartarum (SEQ ID NO: 56 and 57), and the other novel CBGaS enzyme is from the fungal source organism Stachybotrys chlorohalanata (SEQ ID NO: 58). These proteins result in CBGA production that is between 2.24-fold and 3.12-fold the level of Cs.PT4-T. The protein demonstrating the highest CBGaS activity is from Stachybotrys chartarum and is hereafter referred to as Sc.PT (SEQ ID NO: 56). Each of these fungal proteins shares less than 20% sequence similarity to Cs.PT4-T, and the fungal proteins Sb.PT and Sc.PT share 73% pairwise identity.

### Example 12: Identification of SesquiCannaBiGerolic Acid (SCBGA) as a Product Resulting from Substrate Promiscuity of the CBGaS

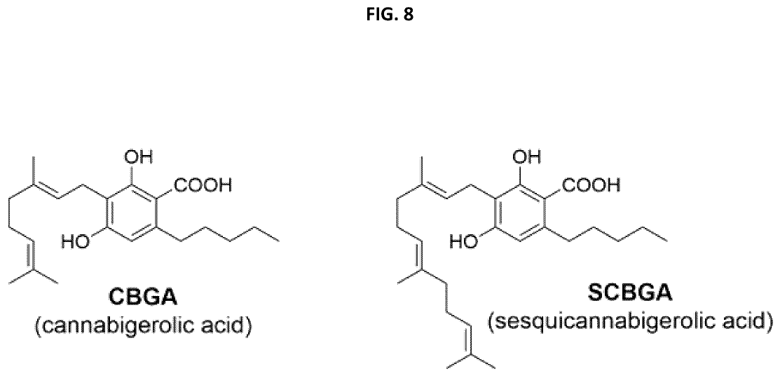

In the course of screening engineered S. cerevisiae strains in Example 11: Identification of Novel Proteins with CBGaS Activity from a Natural Diversity Library, the chromatography assay used to measure CBGA (see Example 3: Analytical Methods for Product Extraction and Titer Determination) indicated that an additional compound with a similar UV-absorbance spectrum was accumulating in the samples. This additional peak eluted from the C18 column at 3.10 min, which is later than the elution time of CBGA (1.28 min) in this assay. Analysis of samples containing this additional peak using high-resolution mass spectroscopy indicated that the compound forms a negative ion with a mass-to-charge ratio of 427.287 m/z, which matches the ion mass of sesquicannabigerolic acid (SCBGA, 427.285 m/z, see FIG. 8). Further fragmentation pattern analysis using tandem high-resolution mass spectrometry provided further support for the assignment of this peak as SCBGA. Finally, upon heating samples containing this additional peak prior to high-resolution mass spectrometry analysis, the peak suspected to be SCBGA disappears and a new peak resembling sesquicannabigerol (SCBG) appears, analogous to the heat-induced decarboxylation of CBGA into CBG. This additional peak occurring in S. cerevisiae strains engineered to produce CBGA is thus reasoned to be SCBGA (FIG. 8).

The chemical structure of SCBGA differs from CBGA by the addition of a C₅H₈ isoprenyl moiety, in the same way that farnesyl pyrophosphate (FPP) differs from geranyl pyrophosphate (GPP). And FPP is an endogenous metabolite of S. cerevisiae, as it is an intermediate in the biosynthesis of ergosterol. The origin of SCBGA in these samples is likely to be from substrate promiscuity of the CBGaS enzyme, whereby it accepts FPP in place of GPP as the prenyl group donor for prenylation of olivetolic acid. In support of this hypothesis, strains differing in the CBGaS enzyme were found to accumulate different ratios of SCBGA and CBGa. The following metric is used in these comparisons:

$$SCBGA fraction = \frac{Area of SCBGA peak in chromatogram}{Sum of the areas of SCBGA and CBGA peaks in chromatogram} \times 100 \%$$

The SCBGA fraction of a strain expressing Cs.PT4-T (SEQ ID NO: 54) was found to be 13.9%. The SCBGA fraction of strains expressing Sb.PT or Sc.PT was found to be 48.3% and 44.3%, respectively (SEQ ID NOS: 55 and 56). These strains are all derived from the same parental strain described in Example 10: Generation of the Base Strain for CBGaS Screening, and hence differ only in the prenyltransferase enzyme. This implies that the novel CBGaS enzymes from fungi are more promiscuous in accepting FPP in place of GPP in the prenylation of olivetolic acid and are thus advantageous for the microbial production of multiple cannabinoids.

### Example 13: Identification of Proteins with Improved CBGaS Substrate Specificity and Activity from Site Saturation Mutagenesis of Sb.PT and Sc.PT

In this example, site-saturation mutagenesis was used to improve the substrate specificity and activity of the novel CBGaS enzymes Sb.PT and Sc.PT (SEQ ID NOS: 55 and 56). Approximately 85% of the amino acid residues in each protein were targeted for mutagenesis, whereby highly conserved residues were deemed essential and avoided during mutagenesis. Each amino acid residue was mutated using the degenerate codon NNT, where “N” indicates any of the four nucleotides. The degenerate codon NNT can encode 15 different amino acids (A, C, D, F, G, H, I, L, N, P, R, S, T, V, and Y). Each library for a given amino acid residue was generated by PCR and transformed into the strain described in Example 10: Generation of the Base Strain for CBGaS Screening.

In primary screening (termed Tier 1), 13 colonies per library were tested using the high-throughput assay described in Example 2: Culturing of Yeast and Example 3: Analytical Methods for Product Extraction and Titer Determination. In a secondary screen (termed Tier 2), transformed strains harboring CBGaS enzyme mutants of interest were re-tested in higher replication (n≥6) to determine if the improved activity was significant. A mutation was considered to improve substrate specificity if the SCBGA fraction produced by the mutant was at least one standard deviation below the median SCBGA fraction of the original CBGaS protein. A mutation was considered to improve activity if the median amount of CBGA produced by the mutant was at least one standard deviation above the median amount of CBGA produced by the original CBGaS protein.

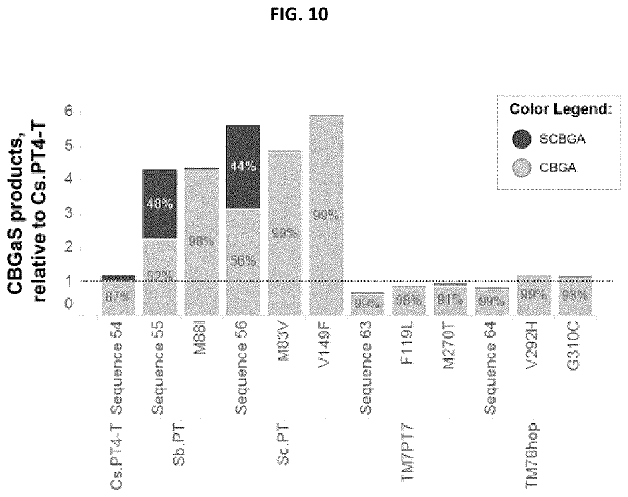

One point mutation in Sb.PT was found to improve both substrate specificity and activity: M88I (Table 13). Remarkably, this single amino acid change reduced the SCBGA fraction from 48.3% to 1.7% (FIG. 10). The CBGaS activity of this mutant was 1.92-fold the level of the original Sb.PT protein. Four other point mutations resulted in improved CBGaS activity (between 1.39-fold and 1.48-fold the level of Sb.PT): V133I, S141Y, Y319L, and L324F. The SCBGA fraction was not measured for the four additional mutants.

TABLE 13
CBGaS Sb.PT sequence and activity data

| | Relative | | SCBGA |
|---|---|---|---|
| Seq ID | CBGA titer | Mutation | Fraction |
| SEQ ID | 0.45 | Reference Enzyme Cs.PT4-T | 13.9% |
| NO 54 | | | |
| SEQ ID | 1.00 | Parent Enzyme Sb.PT | 48.3% |
| NO 55 | | | |
| v1 | 1.92 | M88I | 1.7% |
| v2 | 1.48 | V133I | no data |
| v3 | 1.51 | S141Y | no data |
| v4 | 1.44 | Y319L | no data |
| v5 | 1.39 | L324F | no data |

Two unique point mutations in Sc.PT were found to improve both substrate specificity and activity: M83V and V149F (Table 14). Remarkably, each of these single amino acid changes reduced the SCBGA fraction from 44.3% to 1.4% or 0.8%, respectively (FIG. 10). The CBGaS activity of these mutants compared to the original Sc.PT protein was 1.92-fold and 2.33-fold, respectively.

Seeking to further improve the CBGaS activity, a subsequent campaign of site-saturation mutagenesis was performed, using the top single-point mutant Sc.PT_V149F as the template protein. Approximately 85% of the amino acid residues in the protein were targeted for mutagenesis. Each amino acid residue was mutated using the degenerate codon NNK, where “N” indicates any of the four nucleotides, and “K” indicates either Guanine or Thymine. The degenerate codon NNK can encode all 20 different amino acids (A, C, D, E, F, G, H, I, K, L, M, N, P, Q, R, S, T, V, W, and Y). Each library for a given amino acid residue was generated, transformed, and screening as described above for the original campaign on Sc.PT.

Thirty unique proteins were found to improve CBGaS activity from 3.89-fold to 5.68-fold the level of the original Sc.PT protein. These performance of each of these protein mutants is summarized in Table 14. Notably, the multiple mutations in Sc.PT generally preserved the high substrate specificity afforded by the initial V149F mutation; only the mutant multi_v23 (Table 14) displayed a moderate increase in SCBGA fraction. An enzyme with high activity and specificity for CBGA formation from GPP and olivetolic acid is crucial for the efficient production of CBGA from microbial fermentation. The enzymes identified from fungal species are here demonstrated to possess high activity when expressed in S. cerevisiae, and the point mutations here engineered achieve highest specificity for CBGA formation.

TABLE 14
CBGaS Sc.PT sequence and activity data

| | Relative | | SCBGA |
|---|---|---|---|
| Seq ID | CBGA titer | Mutant | Fraction |
| SEQ ID NO | 0.32 | Reference Enzyme Cs.PT4-T | 13.9% |
| 54 | | | |
| SEQ ID NO | 1.00 | Parent Enzyme Sc.PT | 44.3% |
| 56 | | | |
| single v1 | 1.87 | V149F | 0.8% |
| single v2 | 1.53 | M83V | 1.4% |
| multi v1 | 4.55 | V149F, T202A | <1% |
| multi v2 | 4.27 | V149F, N264Y | <1% |
| multi v3 | 4.21 | V149F, N264F, A282P | <1% |
| multi v4 | 4.13 | V149F, S312L, T11T | <1% |
| multi v5 | 4.06 | V149F, L276T | <1% |
| multi v6 | 3.99 | V149F, L276P | <1% |
| multi v7 | 3.96 | V149F, I324E | <1% |
| multi v8 | 3.91 | V149F, H49C | <1% |
| multi v9 | 3.89 | V149F, H49C | <1% |
| multi v10 | 3.86 | V149F, S312L | <1% |
| multi v11 | 3.86 | V149F, L325P | <1% |
| multi v12 | 3.77 | V149F, I324K | <1% |
| multi v13 | 3.73 | V149F, L325A | <1% |
| multi v14 | 3.68 | V149F, P7K | <1% |
| multi v15 | 3.60 | V149F, R196F | <1% |
| multi v16 | 3.51 | V149F, A176V | <1% |
| multi v17 | 3.49 | V149F, A176V | <1% |
| multi v18 | 3.47 | V149F, N309F | <1% |
| multi v19 | 3.47 | V149F, P7T | <1% |
| multi v20 | 3.43 | V149F, A279C | <1% |
| multi v21 | 3.42 | V149F, A279S | <1% |
| multi v22 | 3.39 | V149F, A89A | <1% |
| multi v23 | 3.31 | V149F, V262L | 1.4% |
| multi v24 | 3.30 | V149F, N93V | <1% |
| multi v25 | 3.25 | V149F, A257Y | <1% |
| multi v26 | 3.21 | V149F, A131G | <1% |
| multi v27 | 3.20 | V149F, A257F, V242L | <1% |
| multi v28 | 3.19 | V149F, C249F | <1% |
| multi v29 | 3.18 | V149F, M311L | <1% |
| multi v30 | 3.11 | V149F, T248A | <1% |
| multi v31 | 1.60 | V149F, M83V | <1% |

### Example 14: Identification of Proteins with Improved CBGaS Substrate Specificity from Chimeragenesis of Cs.PT4-T its and Close Homolog

In this example, protein chimeragenesis is used to improve the substrate specificity of Cs.PT4-T (SEQ ID NO: 54). Protein chimeragenesis part of a family of protein engineering techniques referred to as DNA shuffling, recombination, molecular breeding, simply “chimeragenesis,” or other names (Engqvist M K M & Rabe K S, Plant Physiol. 179:3, 2019, 907-917). In chimeragenesis, new protein sequences are constructed by concatenating different parts of two or more homologous proteins, and the resulting proteins may possess properties not found in any of the parents (Otey C R et al., PLOS Biol. 4:5, 2006, e112). While many proteins generated via chimeragenesis may be non-functional due to protein mis-folding, a careful choice of crossover sites between homologous proteins can result in chimeric proteins that are more likely to be folded and functional (Voigt C A et al., Nat. Struct. Biol., 9:7, 2002, 553-558).

For chimeragenesis of Cs.PT4-T (SEQ ID NO: 54), the four proteins from the previously described homology library (see Example 11: Identification of Novel Proteins with CBGaS Activity from a Natural Diversity Library) that shared the highest pairwise identity with Cs.PT4-T were selected. Two of these homologous proteins are from the source organism Cannabis sativa (SEQ ID NO: 59 and 60), and the other two are from Humulus lupulus (SEQ ID NO: 61 and 62). In previous screening, these four homologs displayed no CBGaS activity, either as full-length proteins or after truncation of the chloroplast transit peptide (cTP). All subsequent work was performed using the cTP-truncated sequences. The four homologs share between 44% and 60% pairwise identity with Cs.PT4-T. Computational prediction of transmembrane (TM) regions of each protein using TMHMM (http://www.cbs.dtu.dk/services/TMHMM/) indicated that all five proteins share a similar domain architecture consisting of nine TM regions.

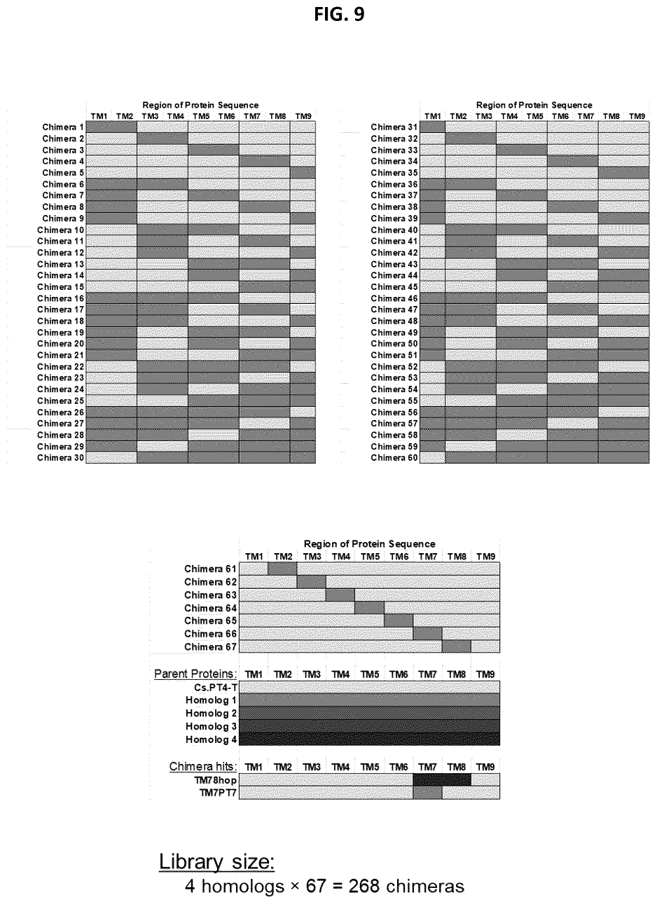

The library of chimeragenesis variants (or simply “chimeras”) was designed as follows. Each chimera consisted of amino acid sequences from exactly two parent proteins, one of which was Cs.PT4-T. The protein sequences were aligned, and crossover sites were selected at the end of each TM region. To facilitate high-efficiency DNA assembly into the landing pad (FIG. 11), the library was constrained to include only chimeras that could be built by five or fewer overlapping DNA pieces. The library of chimeras constructed from Cs.PT4-T and one homolog are illustrated in FIG. 9. The chimeras numbered 1 to 30 consisted of a full-factorial recombination using crossover sites after TM2, TM4, TM6, and TM8. Similarly, the chimeras numbered 31 to 60 consisted of a full-factorial recombination using crossover sites after TM1, TM3, TM5, and TM7. Finally, the chimeras numbered 61 to 67 consisted of more conservative changes to Cs.PT4-T, whereby a single interior TM region was replaced with the corresponding amino acid sequence from a homolog (FIG. 9). The resulting library thus consisted of 67 chimeras using sequence from Cs.PT4-T and a single homolog. Iterating across the four homologs brought the total library size to 4×67=268 chimeras.

Oligonucleotide primers were ordered to construct each chimera in the library. The oligonucleotides added 30-nucleotide overlaps between each piece, as well as 40-nucleotide overlaps to the Landing Pad (FIG. 11), to enable direct transformation into the strain described in Example 10: Generation of the Base Strain for CBGaS Screening. Each chimera was transformed individually, and colony-PCR and Sanger DNA sequencing were used to confirm that the intended chimeras were assembled using in vivo DNA recombination. The resulting strains were screened for the ability to produce CBGA using methods described in Example 2: Culturing of Yeast and Example 3: Analytical Methods for Product Extraction and Titer Determination.

The majority of the 268 chimeras screened in this library resulted in no CBGA production. However, two chimeras were found to produce either 0.59-fold or 0.81-fold the level of CBGA as the reference protein Cs.PT4-T (SEQ ID NO: 54). Notably, both chimeras demonstrated a SCBGA fraction of below 1%, compared to a 13% SCBGA fraction for Cs.PT4-T (Table 15 and FIG. 10). These two hits from chimeragenesis are thus greatly improved in their substrate specificity for GPP over FPP in the prenylation of olivetolic acid. The first chimera, hereafter referred to as TM7PT7 (SEQ ID NO: 63), arises from swapping the TM7 region of Cs.PT4-T with the homologous amino acid sequence from the protein PT7 from Cannabis sativa (SEQ ID NO: 60), and this chimera shares 93% pairwise identity with Cs.PT4-T. The second chimera, hereafter referred to as TM78hop (SEQ ID NO: 64), arises from swapping the TM7 and TM8 regions of Cs.PT4-T with the homologous amino acid sequence from a protein from Humulus lupulus (SEQ ID NO: 62), and this chimera shares 89% pairwise identity with Cs.PT4-T.

TABLE 15
Chimeragenesis sequence and activity data

| | Relative | SCBGa | |
|---|---|---|---|
| Seq ID | CBGa titer | Fraction | CBGaS Protein |
| SEQ ID NO | 1.00 | 12.9% | Reference Enzyme Cs.PT4-T |
| 54 | | | |
| SEQ ID NO | 0.59 | <1.2% | Chimera CBGaS TM7PT7 |
| 63 | | | |
| SEQ ID NO | 0.81 | <1.2% | Chimera CBGaS TM78hop |
| 64 | | | |

### Example 15: Identification of Proteins with Improved CBGaS Activity from Site Saturation Mutagenesis of Two Chimeric Proteins Named TM7PT7 and TM78Hop

The gain of substrate specificity in the two chimeras TM7PT7 (SEQ ID NO: 63) and TM78hop (SEQ ID NO: 64) were accompanied with a reduction in CBGA production, compared to Cs.PT4-T (SEQ ID NO: 54), as summarized in Table 15. In this example, site-saturation mutagenesis was used to improve the CBGaS activity of these two chimeras. Specifically, 91 residues within each protein were mutated. The selected amino acid residues reside within or are spatially adjacent to the transmembrane (TM) regions that differ between these chimeras and Cs.PT4-T. Each amino acid residue was mutated using the degenerate codon NNT, where “N” indicates any of the four nucleotides. The degenerate codon NNT can encode 15 different amino acids (A, C, D, F, G, H, I, L, N, P, R, S, T, V, and Y). Each library for a given amino acid residue was generated by PCR and transformed into the strain described in Example 10: Generation of the Base Strain for CBGaS Screening.

In primary screening (termed Tier 1), 26 colonies per library were tested using the conditions described in Example 2: Culturing of Yeast and the high-throughput assay described in Example 3: Analytical Methods for Product Extraction and Titer Determination. In a secondary screen (termed Tier 2), transformed strains harboring CBGaS enzyme mutants of interest were re-tested in higher replication (n≥6) to determine if the improved activity was significant. A mutation was considered to improve CBGaS activity if the median amount of CBGA produced by the mutant was at least one standard deviation above the median amount of CBGA produced by the starting protein (either TM7PT7 or TM78hop). The SCBGA Fraction of the enzymes was assessed using the approach described in Example 12: Identification of SesquiCannaBiGerolic Acid (SCBGA) as a Product Resulting from Substrate Promiscuity of the CBGaS.

For mutagenesis of TM7PT7, in total ten unique point mutations that improve CBGaS activity were identified (Table 16). These individual mutants resulted in up to 1.31-fold the production of CBGA, compared to the original protein TM7PT7. The SCBGA fraction of these mutants either remained very low (between 1.1% and 2.2%) or increased to levels higher than TM7PT7 but still lower than the reference enzyme Cs.PT4-T (Table 16 and FIG. 10).

TABLE 16
TM7PT7 sequence and activity data

| | Relative | SCBGA | |
|---|---|---|---|
| Seq ID | CBGA titer | Fraction | CBGaS Protein/Mutant |
| SEQ ID NO | 1.00 | 13.5% | Reference Enzyme Cs.PT4-T |
| 54 | | | |
| SEQ ID NO | 0.64 | 1.0% | Parent Enzyme TM7PT7 |
| 63 | | | |
| v1 | 0.80 | 2.1% | I109T |
| v2 | 0.83 | 1.8% | F119L |
| v3 | 0.81 | 4.8% | S245L |
| v4 | 0.73 | 1.1% | S247Y |
| v5 | 0.84 | 9.4% | M270T |
| v6 | 0.72 | 1.9% | S295D |
| v7 | 0.72 | 2.2% | C280L |
| v8 | 0.81 | 7.4% | V314L |
| v9 | 0.72 | 1.5% | A324F |
| v10 | 0.73 | 1.7% | S361I |

For mutagenesis of TM78hop, in total thirteen unique point mutations that improve CBGaS activity were identified (Table 17). These individual mutants resulted in up to 1.51-fold the production of CBGA, compared to the original protein TM78hop. The SCBGA fraction of all these mutants remained low (between 0.8% and 4.3%) (FIG. 10). Notably, seven of these mutations displayed CBGaS activity higher than the reference enzyme Cs.PT4-T and displayed lower SCBGA fraction. For example, the point mutation V292H produced a 1.2% SCBGA fraction, produced 1.15-fold the CBGA compared to Cs.PT4-T, and shares 89% pairwise identity with Cs.PT4-T.

TABLE 17
TM78hop sequence and activity data

| | Relative | SCBGA | |
|---|---|---|---|
| Seq ID | CBGA titer | Fraction | CBGaS Protein/Mutant |
| SEQ ID NO 54 | 1 | 13.5% | Reference Enzyme Cs.PT4-T |
| SEQ ID NO 64 | 0.78 | 1.1% | Parent Enzyme TM78hop |
| v1 | 1.18 | 3.2% | V292Y |
| v2 | 1.15 | 1.2% | V292H |
| v3 | 1.14 | 3.3% | V292F |
| v4 | 1.13 | 3.2% | M275S |
| v5 | 1.10 | 2.2% | G310C |
| v6 | 1.09 | 2.4% | F314N |
| v7 | 1.02 | 1.4% | A347I |
| v8 | 1.00 | 1.3% | M275T |
| v9 | 0.97 | 1.5% | T276C |
| v10 | 0.92 | 0.8% | A331C |
| v11 | 0.90 | 1.8% | T276F |
| v12 | 0.87 | 1.8% | A331T |
| v13 | 0.84 | 4.3% | K291H |

### Example 16: Production of CBGOA, CBGVA, CBGXA, SCBGOA, SCBGVA, and SCBGXA

Cannabigerorcinic acid (CBGOA), cannabigerovarinic acid (CBGVA), 3-geranyl-2,4-dihydroxy-6-phenylethylbenzoic acid (CBGXA), and CBGA differ structurally only in their alkyl side chain. CBGOA has a methyl side chain, CBGVA a propyl side chain, CBGA a pentyl side chain, and CBGXA a phenylethyl side chain. Due to this structural similarity, it was hypothesized that the CBGA-producing enzymes identified in Example 11: Identification of Novel Proteins with CBGaS Activity from a Natural Diversity Library, would also have activity toward production of CBGOA, CBGVA, and/or CBGXA, as well as sesquicannabigerorcinic acid (SCBGOA), sesquicannabigerovarinic acid (SCBGVA), and/or 3-farnesyl-2,4-dihydroxy-6-phenylethylbenzoic acid (SCBGXA).

Saccharomyces cerevisiae strains are transformed with the mevalonate pathway genes (ERG10, ERG13, HMGR-t, ERG12, ERG8, MVD1, and IDI1) and the PDH bypass genes (Zm.PDC, ALD6, and ACS1) and the GPP synthase described in Example 10: Generation of the Base Strain for CBGaS Screening. Each of the prenyltransferases identified in Example 11: Identification of Novel Proteins with CBGaS Activity from a Natural Diversity Library are then expressed in this strain and screened for their ability to produce CBGOA and/or SCBGOA, CBGVA and/or SCBGVA, or CBGXA and/or SCBGXA, as the strains are fed orsellinic acid, divarinolic acid, and 2,4-dihydroxy-6-phenylethylbenzoic acid, respectively. Cells are cultured according to the methods described in Example 2: Culturing Yeast, and using the analytical methods described in Example 3: Analytical Methods for Product Extraction and Titer Determination, CBGOA, SCBGOA, CBGVA, SCBGVA, CBGXA, and SCBGXA are detected and quantified, confirming the ability of the enzymes to act on multiple substrates.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

Exemplary embodiments of the invention are those enumerated below:

1. A host cell capable of producing a cannabinoid, wherein the host cell comprises one or more heterologous nucleic acids that each, independently, encode
(a) an acyl activating enzyme (AAE) having an amino acid sequence that is at least 90% identical to the amino acid sequence of any one of SEQ ID NOS: 1-5 and 7-24, and/or
(b) a tetraketide synthase (TKS) having an amino acid sequence that is at least 90% identical to the amino acid sequence of any one of SEQ ID NOS: 25 and 27-43, and/or
(c) a cannabigerolic acid synthase (CBGaS) having an amino acid sequence that is at least 90% identical to the amino acid sequence of any one of SEQ ID NOS: 55-58, 63, and 64, and/or
(d) an olivetolic acid cyclase (OAC) having an amino acid sequence that is at least 90% identical to the amino acid sequence of any one of SEQ ID NOS: 45-52.

2. The host cell of embodiment 1, wherein the host cell comprises a heterologous nucleic acid that encodes an AAE having an amino acid sequence that is at least 90% identical to the amino acid sequence of any one of SEQ ID NOS: 1-5 and 7-24.

3. The host cell of embodiment 2, wherein the AAE has an amino acid sequence that is at least 95% identical to the amino acid sequence of any one of SEQ ID NOS: 1-5 and 7-24.

4. The host cell of embodiment 3, wherein the AAE has the amino acid sequence of any one of SEQ ID NOS: 1-5 and 7-24.

5. The host cell of embodiment 1, wherein the host cell comprises a heterologous nucleic acid that encodes an AAE having an amino acid sequence that is at least 90% identical to the amino acid sequence of any one of SEQ ID NOS: 1-4.

6. The host cell of embodiment 5, wherein the AAE has an amino acid sequence that is at least 95% identical to the amino acid sequence of any one of SEQ ID NOS: 1-4.

7. The host cell of embodiment 6, wherein the AAE has the amino acid sequence of any one of SEQ ID NOS: 1-4.

8. The host cell of any one of embodiments 1-7, wherein the host cell comprises a heterologous nucleic acid that encodes a TKS having an amino acid sequence that is at least 90% identical to the amino acid sequence of any one of SEQ ID NOS: 25 and 27-43.

9. The host cell of embodiment 8, wherein the TKS has an amino acid sequence that is at least 95% identical to the amino acid sequence of any one of SEQ ID NOS: 25 and 27-43.

10. The host cell of embodiment 9, wherein the TKS has the amino acid sequence of any one of SEQ ID NOS: 25 and 27-43.

11. The host cell of any one of embodiments 1-7, wherein the host cell comprises a heterologous nucleic acid that encodes a TKS having an amino acid sequence that is at least 90% identical to the amino acid sequence of any one of SEQ ID NOS: 25 and 34-39.

12. The host cell of embodiment 11, wherein the TKS has an amino acid sequence that is at least 95% identical to the amino acid sequence of any one of SEQ ID NOS: 25 and 34-39.

13. The host cell of embodiment 12, wherein the TKS has the amino acid sequence of any one of SEQ ID NOS: 25 and 34-39.

14. The host cell of any one of embodiments 1-7, wherein the host cell comprises a heterologous nucleic acid that encodes a TKS having an amino acid sequence that is at least 90% identical to the amino acid sequence of SEQ ID NO: 25 or 39.

15. The host cell of embodiment 14, wherein the TKS has an amino acid sequence that is at least 95% identical to the amino acid sequence of SEQ ID NO: 25 or 39.

16. The host cell of embodiment 15, wherein the TKS has the amino acid sequence of SEQ ID NO: 25 or 39.

17. The host cell of any one of embodiments 1-16, wherein the host cell comprises a heterologous nucleic acid that encodes a CBGaS having an amino acid sequence that is at least 90% identical to the amino acid sequence of any one of SEQ ID NOS: 55-58, 63, and 64.

18. The host cell of embodiment 17, wherein the CBGaS has an amino acid sequence that is at least 95% identical to the amino acid sequence of any one of SEQ ID NOS: 55-58, 63, and 64.

19. The host cell of embodiment 18, wherein the CBGaS has the amino acid sequence of any one of SEQ ID NOS: 55-58, 63, and 64.

20. The host cell of any one of embodiments 17-19, wherein the CBGaS has one or more amino acid substitutions relative to the amino acid sequence of SEQ ID NO: 55, wherein the one or more amino acid substitutions are selected from M88I, V133I, S141Y, Y319L, and L324F.

21. The host cell of embodiment 20, wherein the CBGaS has the amino acid substitution M88I relative to the amino acid sequence of SEQ ID NO: 55.

22. The host cell of embodiment 20 or 21, wherein the CBGaS has the amino acid substitution V133I relative to the amino acid sequence of SEQ ID NO: 55.

23. The host cell of any one of embodiments 20-22, wherein the CBGaS has the amino acid substitution S141Y relative to the amino acid sequence of SEQ ID NO: 55.

24. The host cell of any one of embodiments 20-23, wherein the CBGaS has the amino acid substitution Y319L relative to the amino acid sequence of SEQ ID NO: 55.

25. The host cell of any one of embodiments 20-24, wherein the CBGaS has the amino acid substitution L324F relative to the amino acid sequence of SEQ ID NO: 55.

26. The host cell of any one of embodiments 17-25, wherein the CBGaS has one or more amino acid substitutions relative to the amino acid sequence of SEQ ID NO: 56, wherein the one or more amino acid substitutions are selected from P7K, P7T, T11T, H49C, M83V, A89A, N93V, A131G, V149F, A176V, R196F, T202A, V242L, T248A, C249F, A257Y, A257F, V262L, N264Y, N264F, L276T, L276P, A279C, A279S, A282P, N309F, M311L, S312L, Y319L, I324E, I324K, L325P, and L325A.

27. The host cell of embodiment 26, wherein the CBGaS has the amino acid substitution P7K or P7T relative to the amino acid sequence of SEQ ID NO: 56.

28. The host cell of embodiment 26 or 27, wherein the CBGaS has the amino acid substitution T11T relative to the amino acid sequence of SEQ ID NO: 56.

29. The host cell of any one of embodiments 26-28, wherein the CBGaS has the amino acid substitution H49C relative to the amino acid sequence of SEQ ID NO: 56.

30. The host cell of any one of embodiments 26-29, wherein the CBGaS has the amino acid substitution M83V relative to the amino acid sequence of SEQ ID NO: 56.

31. The host cell of any one of embodiments 26-30, wherein the CBGaS has the amino acid substitution A89A relative to the amino acid sequence of SEQ ID NO: 56.

32. The host cell of any one of embodiments 26-31, wherein the CBGaS has the amino acid substitution N93V relative to the amino acid sequence of SEQ ID NO: 56.

33. The host cell of any one of embodiments 26-32, wherein the CBGaS has the amino acid substitution A131G relative to the amino acid sequence of SEQ ID NO: 56.

34. The host cell of any one of embodiments 26-33, wherein the CBGaS has the amino acid substitution V149F relative to the amino acid sequence of SEQ ID NO: 56.

35. The host cell of any one of embodiments 26-34, wherein the CBGaS has the amino acid substitution A176V relative to the amino acid sequence of SEQ ID NO: 56.

36. The host cell of any one of embodiments 26-35, wherein the CBGaS has the amino acid substitution R196F relative to the amino acid sequence of SEQ ID NO: 56.

37. The host cell of any one of embodiments 26-36, wherein the CBGaS has the amino acid substitution T202A relative to the amino acid sequence of SEQ ID NO: 56.

38. The host cell of any one of embodiments 26-37, wherein the CBGaS has the amino acid substitution V242L relative to the amino acid sequence of SEQ ID NO: 56.

39. The host cell of any one of embodiments 26-38, wherein the CBGaS has the amino acid substitution T248A relative to the amino acid sequence of SEQ ID NO: 56.

40. The host cell of any one of embodiments 26-39, wherein the CBGaS has the amino acid substitution C249F relative to the amino acid sequence of SEQ ID NO: 56.

41. The host cell of any one of embodiments 26-40, wherein the CBGaS has the amino acid substitution A257Y or A257F relative to the amino acid sequence of SEQ ID NO: 56.

42. The host cell of any one of embodiments 26-41, wherein the CBGaS has the amino acid substitution V262L relative to the amino acid sequence of SEQ ID NO: 56.

43. The host cell of any one of embodiments 26-42, wherein the CBGaS has the amino acid substitution N264Y or N264F relative to the amino acid sequence of SEQ ID NO: 56.

44. The host cell of any one of embodiments 26-43, wherein the CBGaS has the amino acid substitution L276T or L276P relative to the amino acid sequence of SEQ ID NO: 56.

45. The host cell of any one of embodiments 26-44, wherein the CBGaS has the amino acid substitution A279C or A279S relative to the amino acid sequence of SEQ ID NO: 56.

46. The host cell of any one of embodiments 26-45, wherein the CBGaS has the amino acid substitution A282P relative to the amino acid sequence of SEQ ID NO: 56.

47. The host cell of any one of embodiments 26-46, wherein the CBGaS has the amino acid substitution N309F relative to the amino acid sequence of SEQ ID NO: 56.

48. The host cell of any one of embodiments 26-47, wherein the CBGaS has the amino acid substitution M311L relative to the amino acid sequence of SEQ ID NO: 56.

49. The host cell of any one of embodiments 26-48, wherein the CBGaS has the amino acid substitution S312L relative to the amino acid sequence of SEQ ID NO: 56.

50. The host cell of any one of embodiments 26-49, wherein the CBGaS has the amino acid substitution Y319L relative to the amino acid sequence of SEQ ID NO: 56.

51. The host cell of any one of embodiments 26-50, wherein the CBGaS has the amino acid substitution I324E or I324K relative to the amino acid sequence of SEQ ID NO: 56.

52. The host cell of any one of embodiments 26-51, wherein the CBGaS has the amino acid substitution L325P or L325A relative to the amino acid sequence of SEQ ID NO: 56.

53. The host cell of any one of embodiments 17-52, wherein the CBGaS has one or more amino acid substitutions relative to the amino acid sequence of SEQ ID NO: 63, wherein the one or more amino acid substitutions are selected from I109T, F119L, S245L, S247Y, M270T, C280L, S295D, V314L, A324F, and S361I.

54. The host cell of embodiment 53, wherein the CBGaS has the amino acid substitution I109T relative to the amino acid sequence of SEQ ID NO: 63.

55. The host cell of embodiment 53 or 54, wherein the CBGaS has the amino acid substitution F119L relative to the amino acid sequence of SEQ ID NO: 63.

56. The host cell of any one of embodiments 53-55, wherein the CBGaS has the amino acid substitution S245L relative to the amino acid sequence of SEQ ID NO: 63.

57. The host cell of any one of embodiments 53-56, wherein the CBGaS has the amino acid substitution S247Y relative to the amino acid sequence of SEQ ID NO: 63.

58. The host cell of any one of embodiments 53-57, wherein the CBGaS has the amino acid substitution M270T relative to the amino acid sequence of SEQ ID NO: 63.

59. The host cell of any one of embodiments 53-58, wherein the CBGaS has the amino acid substitution C280L relative to the amino acid sequence of SEQ ID NO: 63.

60. The host cell of any one of embodiments 53-59, wherein the CBGaS has the amino acid substitution S295D relative to the amino acid sequence of SEQ ID NO: 63.

61. The host cell of any one of embodiments 53-60, wherein the CBGaS has the amino acid substitution V314L relative to the amino acid sequence of SEQ ID NO: 63.

62. The host cell of any one of embodiments 53-61, wherein the CBGaS has the amino acid substitution A324F relative to the amino acid sequence of SEQ ID NO: 63.

63. The host cell of any one of embodiments 53-62, wherein the CBGaS has the amino acid substitution S361I relative to the amino acid sequence of SEQ ID NO: 63.

64. The host cell of any one of embodiments 17-63, wherein the CBGaS has one or more amino acid substitutions relative to the amino acid sequence of SEQ ID NO: 64, wherein the one or more amino acid substitutions are selected from M275S, M275T, T276C, T276F, K291H, V292Y, V292H, V292F, G310C, F314N, A331C, A331T, and A347I.

65. The host cell of embodiment 64, wherein the CBGaS has the amino acid substitution M275S or M275T relative to the amino acid sequence of SEQ ID NO: 64.

66. The host cell of embodiment 64 or 65, wherein the CBGaS has the amino acid substitution T276C or T276F relative to the amino acid sequence of SEQ ID NO: 64.

67. The host cell of any one of embodiments 64-66, wherein the CBGaS has the amino acid substitution K291H relative to the amino acid sequence of SEQ ID NO: 64.

68. The host cell of any one of embodiments 64-67, wherein the CBGaS has the amino acid substitution V292Y, V292H, or V292F relative to the amino acid sequence of SEQ ID NO: 64.

69. The host cell of any one of embodiments 64-68, wherein the CBGaS has the amino acid substitution G310C relative to the amino acid sequence of SEQ ID NO: 64.

70. The host cell of any one of embodiments 64-69, wherein the CBGaS has the amino acid substitution F314N relative to the amino acid sequence of SEQ ID NO: 64.

71. The host cell of any one of embodiments 64-70, wherein the CBGaS has the amino acid substitution A331C or A331T relative to the amino acid sequence of SEQ ID NO: 64.

72. The host cell of any one of embodiments 64-71, wherein the CBGaS has the amino acid substitution A347 I relative to the amino acid sequence of SEQ ID NO: 64.

73. The host cell of any one of embodiments 1-72, wherein the host cell comprises a heterologous nucleic acid that encodes an OAC having an amino acid sequence that is at least 90% identical to the amino acid sequence of any one of SEQ ID NOS: 45-52.

74. The host cell of embodiment 73, wherein the OAC has an amino acid sequence that is at least 95% identical to the amino acid sequence of any one of SEQ ID NOS: 45-52.

75. The host cell of embodiment 74, wherein OAC has the amino acid sequence of any one of SEQ ID NOS: 45-52.

76. The host cell of any one of embodiments 73-75, wherein the OAC has one or more amino acid substitutions relative to the amino acid sequence of SEQ ID NO: 44, wherein the one or more amino acid substitutions are selected from A2S, L91, K12S, E14S, F23L, V28L, T47R, Q48R, K49R, S87H, F88Y, and L92Y.

77. The host cell of embodiment 76, wherein the OAC has the amino acid substitution A2S relative to the amino acid sequence of SEQ ID NO: 44.

78. The host cell of embodiment 76 or 77, wherein the OAC has the amino acid substitution L91 relative to the amino acid sequence of SEQ ID NO: 44.

79. The host cell of any one of embodiments 76-78, wherein the OAC has the amino acid substitution K12S relative to the amino acid sequence of SEQ ID NO: 44.

80. The host cell of any one of embodiments 76-79, wherein the OAC has the amino acid substitution E14S relative to the amino acid sequence of SEQ ID NO: 44.

81. The host cell of any one of embodiments 76-80, wherein the OAC has the amino acid substitution F23L relative to the amino acid sequence of SEQ ID NO: 44.

82. The host cell of any one of embodiments 76-81, wherein the OAC has the amino acid substitution V28L relative to the amino acid sequence of SEQ ID NO: 44.

83. The host cell of any one of embodiments 76-82, wherein the OAC has the amino acid substitution T47R relative to the amino acid sequence of SEQ ID NO: 44.

84. The host cell of any one of embodiments 76-83, wherein the OAC has the amino acid substitution Q48R relative to the amino acid sequence of SEQ ID NO: 44.

85. The host cell of any one of embodiments 76-84, wherein the OAC has the amino acid substitution K49R relative to the amino acid sequence of SEQ ID NO: 44.

86. The host cell of any one of embodiments 76-85, wherein the OAC has the amino acid substitution S87H relative to the amino acid sequence of SEQ ID NO: 44.

87. The host cell of any one of embodiments 76-86, wherein the OAC has the amino acid substitution F88Y relative to the amino acid sequence of SEQ ID NO: 44.

88. The host cell of any one of embodiments 76-87, wherein the OAC has the amino acid substitution L92Y relative to the amino acid sequence of SEQ ID NO: 44.

89. The host cell of any one of embodiments 1-88, wherein the host cell further comprises one or more heterologous nucleic acids that each, independently, encode an enzyme of the mevalonate biosynthetic pathway.

90. The host cell of embodiment 89, wherein the enzyme of the mevalonate biosynthetic pathway is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

91. The host cell of embodiment 89 or 90, wherein the host cell comprises one or more heterologous nucleic acids that, together, encode an acetyl-CoA thiolase, an HMG-COA synthase, an HMG-COA reductase, a mevalonate kinase, a phosphomevalonate kinase, a mevalonate pyrophosphate decarboxylase, and an IPP:DMAPP isomerase.

92. The host cell of embodiment 90 or 91, wherein the acetyl-CoA thiolase has an amino acid sequence that is at least 90% identical to the amino acid sequence of SEQ ID NO: 68.

93. The host cell of embodiment 92, wherein the acetyl-CoA thiolase has an amino acid sequence that is at least 95% identical to the amino acid sequence of SEQ ID NO: 68.

94. The host cell of embodiment 93, wherein the acetyl-CoA thiolase has the amino acid sequence of SEQ ID NO: 68.

95. The host cell of any one of embodiments 90-94, wherein the HMG-COA synthase has an amino acid sequence that is at least 90% identical to the amino acid sequence of SEQ ID NO: 69.

96. The host cell of embodiment 95, wherein the HMG-COA synthase has an amino acid sequence that is at least 95% identical to the amino acid sequence of SEQ ID NO: 69.

97. The host cell of embodiment 96, wherein the HMG-COA synthase has the amino acid sequence of SEQ ID NO: 69.

98. The host cell of any one of embodiments 90-97, wherein the HMG-COA reductase has an amino acid sequence that is at least 90% identical to the amino acid sequence of SEQ ID NO: 70.

99. The host cell of embodiment 98, wherein the HMG-COA reductase has an amino acid sequence that is at least 95% identical to the amino acid sequence of SEQ ID NO: 70.

100. The host cell of embodiment 99, wherein the HMG-COA reductase has the amino acid sequence of SEQ ID NO: 70.

101. The host cell of any one of embodiments 90-100, wherein the mevalonate kinase has an amino acid sequence that is at least 90% identical to the amino acid sequence of SEQ ID NO: 71.

102. The host cell of embodiment 101, wherein the mevalonate kinase has an amino acid sequence that is at least 95% identical to the amino acid sequence of SEQ ID NO: 71.

103. The host cell of embodiment 102, wherein the mevalonate kinase has the amino acid sequence of SEQ ID NO: 71.

104. The host cell of any one of embodiments 90-103, wherein the phosphomevalonate kinase has an amino acid sequence that is at least 90% identical to the amino acid sequence of SEQ ID NO: 72.

105. The host cell of embodiment 104, wherein the phosphomevalonate kinase has an amino acid sequence that is at least 95% identical to the amino acid sequence of SEQ ID NO: 72.

106. The host cell of embodiment 105, wherein the phosphomevalonate kinase has the amino acid sequence of SEQ ID NO: 72.

107. The host cell of any one of embodiments 90-106, wherein the mevalonate pyrophosphate decarboxylase has an amino acid sequence that is at least 90% identical to the amino acid sequence of SEQ ID NO: 73.

108. The host cell of embodiment 107, wherein the mevalonate pyrophosphate decarboxylase has an amino acid sequence that is at least 95% identical to the amino acid sequence of SEQ ID NO: 73.

109. The host cell of embodiment 108, wherein the mevalonate pyrophosphate decarboxylase has the amino acid sequence of SEQ ID NO: 73.

110. The host cell of any one of embodiments 90-109, wherein the IPP:DMAPP isomerase has an amino acid sequence that is at least 90% identical to the amino acid sequence of SEQ ID NO: 74.

111. The host cell of embodiment 110, wherein the IPP:DMAPP isomerase has an amino acid sequence that is at least 95% identical to the amino acid sequence of SEQ ID NO: 74.

112. The host cell of embodiment 111, wherein the IPP:DMAPP isomerase has the amino acid sequence of SEQ ID NO: 74.

113. The host cell of any one of embodiments 1-112, wherein the host cell further comprises a heterologous nucleic acid that encodes a geranyl pyrophosphate (GPP) synthase.

114. The host ell of embodiment 113, wherein the GPP synthase has an amino acid sequence that is at least 90% identical to the amino acid sequence of SEQ ID NO: 75.

115. The host ell of embodiment 114, wherein the GPP synthase has an amino acid sequence that is at least 95% identical to the amino acid sequence of SEQ ID NO: 75.

116. The host ell of embodiment 115, wherein the GPP synthase has the amino acid sequence of SEQ ID NO: 75.

117. The host cell of any one of embodiments 1-116, wherein the host cell further comprises one or more heterologous nucleic acids that each, independently, encode an acetyl-CoA synthase, an aldehyde dehydrogenase, and/or a pyruvate decarboxylase.

118. The host cell of embodiment 117, wherein the host cell comprises one or more heterologous nucleic acids that, together, encode an acetyl-CoA synthase, an aldehyde dehydrogenase, and a pyruvate decarboxylase.

119. The host cell of embodiment 117 or 118, wherein the acetyl-CoA synthase has an amino acid sequence that is at least 90% identical to the amino acid sequence of SEQ ID NO: 66.

120. The host cell of embodiment 119, wherein the acetyl-CoA synthase has an amino acid sequence that is at least 95% identical to the amino acid sequence of SEQ ID NO: 66.

121. The host cell of embodiment 120, wherein the acetyl-CoA synthase has the amino acid sequence of SEQ ID NO: 66.

122. The host cell of any one of embodiments 117-121, wherein the aldehyde dehydrogenase has an amino acid sequence that is at least 90% identical to the amino acid sequence of SEQ ID NO: 67.

123. The host cell of embodiment 122, wherein the aldehyde dehydrogenase has an amino acid sequence that is at least 95% identical to the amino acid sequence of SEQ ID NO: 67.

124. The host cell of embodiment 123, wherein the aldehyde dehydrogenase has the amino acid sequence of SEQ ID NO: 67.

125. The host cell of any one of embodiments 117-124, wherein the pyruvate decarboxylase has an amino acid sequence that is at least 90% identical to the amino acid sequence of SEQ ID NO: 65.

126. The host cell of embodiment 125, wherein the pyruvate decarboxylase has an amino acid sequence that is at least 95% identical to the amino acid sequence of SEQ ID NO: 65.

127. The host cell of embodiment 126, wherein the pyruvate decarboxylase has the amino acid sequence of SEQ ID NO: 65.

128. The host cell of any one of embodiments 1-127, wherein the host cell comprises heterologous nucleic acids that independently encode
(a) an AAE having the amino acid sequence of any one of SEQ ID NOS: 1-5 and 7-24,
(b) a TKS having the amino acid sequence of any one of SEQ ID NOS: 25 and 27-43,
(c) a CBGaS having the amino acid sequences of any one of SEQ ID NOS: 55-58, 63, and 64, and
(d) an OAC having the amino acid sequence of any one of SEQ ID NO: 45-52.

129. The host cell of any one of embodiments 1-128, wherein expression of one or more of the heterologous nucleic acids is regulated by an exogenous agent.

130. The host cell of embodiment 129, wherein the exogenous agent decreases production of the cannabinoid.

131. The host cell of embodiment 129, wherein the exogenous agent increases production of the cannabinoid.

132. The host cell of embodiment 131, wherein the exogenous agent is galactose and expression of one or more of the heterologous nucleic acids is under the control of a GAL promoter.

133. The host cell of embodiment 129, wherein expression of one or more of the heterologous nucleic acids is under the control of a galactose-responsive promoter, a maltose-responsive promoter, or a combination of both.

134. The host cell of any one of embodiments 1-133, wherein the cannabinoid is cannabigerolic acid (CBGA), cannabigerol (CBG), sesquicannabigerolic acid (SCBGA), cannabigerorcinic acid (CBGOA), sesquicannabigerorcinic acid (SCBGOA), cannabigerovarinic acid (CBGVA), sesquicannabigerovarinic acid (SCBGVA), 3-geranyl-2,4-dihydroxy-6-phenylethylbenzoic acid (CBGXA), or 3-farnesyl-2,4-dihydroxy-6-phenylethylbenzoic acid (SCBGXA).

135. The host cell of any one of embodiments 1-134, wherein the host cell is a yeast cell or yeast strain.

136. The host cell of embodiment 135, wherein the yeast cell is S. cerevisiae.

137. A mixture comprising the host cell of any one of embodiments 1-136 and a culture medium.

138. The mixture of embodiment 137, wherein the culture medium comprises an exogenous agent that decreases production of the cannabinoid.

139. The mixture of embodiment 138, wherein the exogenous agent is maltose.

140. The mixture of embodiment 137, wherein the culture medium comprises (i) an exogenous agent that increases production of the cannabinoid, and (ii) a precursor required to make the cannabinoid.

141. The mixture of embodiment 140, wherein the exogenous agent is galactose.

142. The mixture of embodiment 140 or 141, wherein the precursor required to make the cannabinoid is hexanoate.

143. A method for decreasing the expression of a cannabinoid, the method comprising culturing the host cell of any one of embodiments 1-136 in a medium comprising an exogenous agent, wherein the exogenous agent decreases the expression of the cannabinoid.

144. The method of embodiment 143, wherein the exogenous agent is maltose.

145. The method of embodiment 143 or 144, wherein culturing the host cell in the medium comprising the exogenous agent results in less than 0.001 mg/L of cannabinoid.

146. A method for increasing the expression of cannabinoid, the method comprising culturing the host cell of any one of embodiments 1-136 in a medium comprising an exogenous agent, wherein the exogenous agent increases expression of the cannabinoid.

147. The method of embodiment 146, wherein the exogenous agent is galactose.

148. The method of embodiment 146 or 147, further comprising culturing the host cell with a precursor required to make the cannabinoid.

149. The method of embodiment 148, wherein the precursor required to make the cannabinoid is hexanoate.

150. A method of genetically modifying a host cell to be capable of producing a cannabinoid, the method comprising introducing into the host cell one or more heterologous nucleic acids that each, independently, encode
(a) an AAE having an amino acid sequence that is at least 90% identical to the amino acid sequence of any one of SEQ ID NOS: 1-5 and 7-24, and/or
(b) a TKS having an amino acid sequence that is at least 90% identical to the amino acid sequence of any one of SEQ ID NOS: 25 and 27-43, and/or
(c) a CBGaS having an amino acid sequence that is at least 90% identical to the amino acid sequence of any one of SEQ ID NOS: 55-58, 63, and 64, and/or
(d) an OAC having an amino acid sequence that is at least 90% identical to the amino acid sequence of any one of SEQ ID NO: 45-52.

151. The method of embodiment 150, wherein the method comprises introducing into the host cell a heterologous nucleic acid that encodes an AAE having an amino acid sequence that is at least 90% identical to the amino acid sequence of any one of SEQ ID NOS: 1-5 and 7-24.

152. The method of embodiment 151, wherein the AAE has an amino acid sequence that is at least 95% identical to the amino acid sequence of any one of SEQ ID NOS: 1-5 and 7-24.

153. The method of embodiment 152, wherein the AAE has the amino acid sequence of any one of SEQ ID NOS: 1-5 and 7-24.

154. The method of any one of embodiments 150-153, wherein the method comprises introducing into the host cell a heterologous nucleic acid that encodes a TKS having an amino acid sequence that is at least 90% identical to the amino acid sequence of any one of SEQ ID NOS: 25 and 27-43.

155. The method of embodiment 154, wherein the TKS has an amino acid sequence that is at least 95% identical to the amino acid sequence of any one of SEQ ID NOS: 25 and 27-43.

156. The method of embodiment 155, wherein the TKS has the amino acid sequence of any one of SEQ ID NOS: 25 and 27-43.

157. The method of any one of embodiments 150-156, wherein the method comprises introducing into the host cell a heterologous nucleic acid that encodes a CBGaS having an amino acid sequence that is at least 90% identical to the amino acid sequence of any one of SEQ ID NOS: 55-58, 63, and 64.

158. The method of embodiment 157, wherein the CBGaS has an amino acid sequence that is at least 95% identical to the amino acid sequence of any one of SEQ ID NOS: 55-58, 63, and 64.

159. The method of embodiment 158, wherein the CBGaS has the amino acid sequence of any one of SEQ ID NOS: 55-58, 63, and 64.

160. The method of any one of embodiments 150-159, wherein the method comprises introducing into the host cell a heterologous nucleic acid that encodes a OAC having an amino acid sequence that is at least 90% identical to the amino acid sequence of any one of SEQ ID NOS: 45-52.

161. The method of embodiment 160, wherein the OAC has an amino acid sequence that is at least 95% identical to the amino acid sequence of any one of SEQ ID NOS: 45-52.

162. The method of embodiment 161, wherein OAC has the amino acid sequence of any one of SEQ ID NOS: 45-52.

163. The method of any one of embodiments 150-162, wherein the host cell comprises one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

164. The method of embodiment 163, wherein the host cell comprises one or more heterologous nucleic acids that, together, encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

165. The method of embodiment 163 or 164, wherein the acetyl-CoA thiolase has an amino acid sequence that is at least 90% identical to the amino acid sequence of SEQ ID NO: 68, optionally wherein the acetyl-CoA thiolase has an amino acid sequence that is at least 95% identical to the amino acid sequence of SEQ ID NO: 68, optionally wherein the acetyl-CoA thiolase has the amino acid sequence of SEQ ID NO: 68.

166. The method of any one of embodiments 163-165, wherein the HMG-COA synthase has an amino acid sequence that is at least 90% identical to the amino acid sequence of SEQ ID NO: 69 optionally wherein the HMG-COA synthase has an amino acid sequence that is at least 95% identical to the amino acid sequence of SEQ ID NO: 69, optionally wherein the HMG-COA synthase has the amino acid sequence of SEQ ID NO: 69.

167. The method of any one of embodiments 163-166, wherein the HMG-COA reductase has an amino acid sequence that is at least 90% identical to the amino acid sequence of SEQ ID NO: 70, optionally wherein the HMG-COA reductase has an amino acid sequence that is at least 95% identical to the amino acid sequence of SEQ ID NO: 70, optionally wherein the HMG-COA reductase has the amino acid sequence of SEQ ID NO: 70.

168. The method of any one of embodiments 163-167, wherein the mevalonate kinase has an amino acid sequence that is at least 90% identical to the amino acid sequence of SEQ ID NO: 71, optionally wherein the mevalonate kinase has an amino acid sequence that is at least 95% identical to the amino acid sequence of SEQ ID NO: 71, optionally wherein the mevalonate kinase has the amino acid sequence of SEQ ID NO: 71.

169. The method of any one of embodiments 163-168, wherein the phosphomevalonate kinase has an amino acid sequence that is at least 90% identical to the amino acid sequence of SEQ ID NO: 72, optionally wherein the phosphomevalonate kinase has an amino acid sequence that is at least 95% identical to the amino acid sequence of SEQ ID NO: 72, optionally wherein the phosphomevalonate kinase has the amino acid sequence of SEQ ID NO: 72.

170. The method of any one of embodiments 163-169, wherein the mevalonate pyrophosphate decarboxylase has an amino acid sequence that is at least 90% identical to the amino acid sequence of SEQ ID NO: 73, optionally wherein the mevalonate pyrophosphate decarboxylase has an amino acid sequence that is at least 95% identical to the amino acid sequence of SEQ ID NO: 73, optionally wherein the mevalonate pyrophosphate decarboxylase has the amino acid sequence of SEQ ID NO: 73.

171. The method of any one of embodiments 163-170, wherein the IPP:DMAPP isomerase has an amino acid sequence that is at least 90% identical to the amino acid sequence of SEQ ID NO: 74, optionally wherein the IPP:DMAPP isomerase has an amino acid sequence that is at least 95% identical to the amino acid sequence of SEQ ID NO: 74, optionally wherein the IPP:DMAPP isomerase has the amino acid sequence of SEQ ID NO: 74.

172. The method of any one of embodiments 150-171, wherein the host cell comprises a heterologous nucleic acid that encodes a GPP synthase.

173. The method of embodiment 172, wherein the GPP synthase has an amino acid sequence that is at least 90% identical to the amino acid sequence of SEQ ID NO: 75, optionally wherein the GPP synthase has an amino acid sequence that is at least 95% identical to the amino acid sequence of SEQ ID NO: 75, optionally wherein the GPP synthase has the amino acid sequence of SEQ ID NO: 75.

174. The method of any one of embodiments 150-173, wherein the host cell comprises one or more heterologous nucleic acids that each, independently, encode an acetyl-CoA synthase, an aldehyde dehydrogenase, and/or a pyruvate decarboxylase.

175. The method of embodiment 174, wherein the host cell comprises one or more heterologous nucleic acids that, together, encode an acetyl-CoA synthase, an aldehyde dehydrogenase, and a pyruvate decarboxylase.

176. The method of embodiment 174 or 175, wherein the acetyl-CoA synthase has an amino acid sequence that is at least 90% identical to the amino acid sequence of SEQ ID NO: 66, optionally wherein the acetyl-CoA synthase has an amino acid sequence that is at least 95% identical to the amino acid sequence of SEQ ID NO: 66, optionally wherein the acetyl-CoA synthase has the amino acid sequence of SEQ ID NO: 66.

177. The method of any one of embodiments 174-176, wherein the aldehyde dehydrogenase has an amino acid sequence that is at least 90% identical to the amino acid sequence of SEQ ID NO: 67, optionally wherein the aldehyde dehydrogenase has an amino acid sequence that is at least 95% identical to the amino acid sequence of SEQ ID NO: 67, optionally wherein the aldehyde dehydrogenase has the amino acid sequence of SEQ ID NO: 67.

178. The method of any one of embodiments 174-177, wherein the pyruvate decarboxylase has an amino acid sequence that is at least 90% identical to the amino acid sequence of SEQ ID NO: 65, optionally wherein the pyruvate decarboxylase has an amino acid sequence that is at least 95% identical to the amino acid sequence of SEQ ID NO: 65, optionally wherein the pyruvate decarboxylase has the amino acid sequence of SEQ ID NO: 65.

179. The method of any one of embodiments 150-178, wherein one or more of the heterologous nucleic acids are regulated by an exogenous agent.

180. The method of any one of embodiments 150-179, wherein expression of one or more of the heterologous nucleic acids is regulated by an exogenous agent.

181. The method of embodiment 180, wherein the exogenous agent decreases production of the cannabinoid.

182. The method of embodiment 180, wherein the exogenous agent increases production of the cannabinoid.

183. The method of embodiment 182, wherein the exogenous agent is galactose and expression of one or more of the heterologous nucleic acids is under the control of a GAL promoter.

184. The method of embodiment 180, wherein expression of one or more of the heterologous nucleic acids is under the control of a galactose-responsive promoter, a maltose-responsive promoter, or a combination of both.

185. The method of any one of embodiments 150-184, wherein the cannabinoid is CBGA, CBG, SCBGA, CBGOA, SCBGOA, CBGVA, SCBGVA, CBGXA, or SCBGXA.

186. The method of any one of embodiments 150-185, wherein the host cell is a yeast cell or yeast strain.

187. The method of embodiment 186, wherein the yeast cell is S. cerevisiae.

188. A method of producing a cannabinoid, the method comprising culturing a population of genetically modified host cells of any one of embodiments 1-136 in a culture medium under conditions suitable for the host cells to produce the cannabinoid.

189. The method of embodiment 188, wherein the culture medium comprises less than 3 mM hexanoic acid.

190. A fermentation composition comprising (i) a population of genetically modified yeast cells comprising the host cell of any one of embodiments 1-136 and (ii) a culture medium comprising one or more cannabinoids produced from the yeast cells.

191. A method of recovering one or more cannabinoids from the fermentation composition of embodiment 190, the method comprising:
(i) separating at least a portion of the population of genetically modified yeast cells from the culture medium; and
(ii) contacting the separated host cells with a wash liquid; and
(iii) removing the wash liquid from the separated host cells.

192. A method of producing a cannabinoid, the method comprising culturing the mixture of any one of embodiments 137-142 under conditions suitable for the host cells to produce the cannabinoid.

193. A fermentation composition comprising a mixture of any one of embodiments 137-142.

194. A non-naturally occurring CBGaS enzyme capable of producing CBGA and at least one additional cannabinoid selected from SCBGA, CBGOA, SCBGOA, CBGVA, SCBGVA, CBGXA, and SCBGXA.

195. A non-naturally occurring CBGaS enzyme capable of accepting, as a substrate, olivetolic acid and at least one additional precursor selected from orsellinic acid, divarinolic acid, and 2,4-dihydroxy-6-phenylethylbenzoic acid.

196. A non-naturally occurring CBGaS enzyme capable of catalyzing:
(a) conversion of olivetolic acid to cannabigerolic acid (CBGA) in the presence of GPP and/or to sesquicannabigerolic acid (SCBGA) in the presence of FPP; and/or
(b) conversion of orsellinic acid to cannabigerorcinic acid (CBGOA) in the presence of GPP and/or to sesquicannabigerorcinic acid (SCBGOA) in the presence of FPP; and/or
(c) conversion of divarinolic acid to cannabigerovarinic acid (CBGVA) in the presence of GPP and/or to sesquicannabigerovarinic acid (SCBGVA) in the presence of FPP; and/or
(d) conversion of 2,4-dihydroxy-6-phenylethylbenzoic acid to 3-geranyl-2,4-dihydroxy-6-phenylethylbenzoic acid (CBGXA) in the presence of GPP and/or to 3-farnesyl-2,4-dihydroxy-6-phenylethylbenzoic acid (SCBGXA) in the presence of FPP.

197. A non-naturally occurring CBGaS enzyme having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 55, SEQ ID NO: 56, SEQ ID NO:57, or SEQ ID NO:58.

198. The CBGaS enzyme of any one of embodiments 194-197, wherein the CBGaS comprises one or more amino acid substitutions relative to the amino acid sequence of SEQ ID NO: 55 selected from M88I, V133I, S141Y, Y319L, and L324F.

199. The CBGaS enzyme of any one of embodiments 194-198, wherein the CBGaS has the amino acid substitution M88I relative to the amino acid sequence of SEQ ID NO: 55.

200. The CBGaS enzyme of any one of embodiments 194-199, wherein the CBGaS has the amino acid substitution V133I relative to the amino acid sequence of SEQ ID NO: 55.

201. The CBGaS enzyme of any one of embodiments 194-200, wherein the CBGaS has the amino acid substitution S141Y relative to the amino acid sequence of SEQ ID NO: 55.

202. The CBGaS enzyme of any one of embodiments 194-201, wherein the CBGaS has the amino acid substitution Y319L relative to the amino acid sequence of SEQ ID NO: 55.

203. The CBGaS enzyme of any one of embodiments 194-202, wherein the CBGaS has the amino acid substitution L324F relative to the amino acid sequence of SEQ ID NO: 55.

204. The CBGaS enzyme of any one of embodiments 194-197, wherein the CBGaS comprises one or more amino acid substitutions relative to the amino acid sequence of SEQ ID NO: 56 selected from P7K, P7T, T11T, H49C, M83V, A89A, N93V, A131G, V149F, A176V, R196F, T202A, V242L, T248A, C249F, A257Y, A257F, V262L, N264Y, N264F, L276T, L276P, A279C, A279S, A282P, N309F, M311L, S312L, Y319L, I324E, I324K, L325P, and L325A.

205. The CBGaS enzyme of any one of embodiments 194-197 and 204, wherein the CBGaS has the amino acid substitution P7K or P7T relative to the amino acid sequence of SEQ ID NO: 56.

206. The CBGaS enzyme of any one of embodiments 194-197, 204, and 205, wherein the CBGaS has the amino acid substitution T11T relative to the amino acid sequence of SEQ ID NO: 56.

207. The CBGaS enzyme of any one of embodiments 194-197 and 204-206, wherein the CBGaS has the amino acid substitution H49C relative to the amino acid sequence of SEQ ID NO: 56.

208. The CBGaS enzyme of any one of embodiments 194-197 and 204-207, wherein the CBGaS has the amino acid substitution M83V relative to the amino acid sequence of SEQ ID NO: 56.

209. The CBGaS enzyme of any one of embodiments 194-197 and 204-208, wherein the CBGaS has the amino acid substitution A89A relative to the amino acid sequence of SEQ ID NO: 56.

210. The CBGaS enzyme of any one of embodiments 194-197 and 204-209, wherein the CBGaS has the amino acid substitution N93V relative to the amino acid sequence of SEQ ID NO: 56.

211. The CBGaS enzyme of any one of embodiments 194-197 and 204-210, wherein the CBGaS has the amino acid substitution A131G relative to the amino acid sequence of SEQ ID NO: 56.

212. The CBGaS enzyme of any one of embodiments 194-197 and 204-211, wherein the CBGaS has the amino acid substitution V149F relative to the amino acid sequence of SEQ ID NO: 56.

213. The CBGaS enzyme of any one of embodiments 194-197 and 204-212, wherein the CBGaS has the amino acid substitution A176V relative to the amino acid sequence of SEQ ID NO: 56.

214. The CBGaS enzyme of any one of embodiments 194-197 and 204-213, wherein the CBGaS has the amino acid substitution R196F relative to the amino acid sequence of SEQ ID NO: 56.

215. The CBGaS enzyme of any one of embodiments 194-197 and 204-214, wherein the CBGaS has the amino acid substitution T202A relative to the amino acid sequence of SEQ ID NO: 56.

216. The CBGaS enzyme of any one of embodiments 194-197 and 204-215, wherein the CBGaS has the amino acid substitution V242L relative to the amino acid sequence of SEQ ID NO: 56.

217. The CBGaS enzyme of any one of embodiments 194-197 and 204-216, wherein the CBGaS has the amino acid substitution T248A relative to the amino acid sequence of SEQ ID NO: 56.

218. The CBGaS enzyme of any one of embodiments 194-197 and 204-217, wherein the CBGaS has the amino acid substitution C249F relative to the amino acid sequence of SEQ ID NO: 56.

219. The CBGaS enzyme of any one of embodiments 194-197 and 204-218, wherein the CBGaS has the amino acid substitution A257Y or A257F relative to the amino acid sequence of SEQ ID NO: 56.

220. The CBGaS enzyme of any one of embodiments 194-197 and 204-219, wherein the CBGaS has the amino acid substitution V262L relative to the amino acid sequence of SEQ ID NO: 56.

221. The CBGaS enzyme of any one of embodiments 194-197 and 204-220, wherein the CBGaS has the amino acid substitution N264Y or N264F relative to the amino acid sequence of SEQ ID NO: 56.

222. The CBGaS enzyme of any one of embodiments 194-197 and 204-221, wherein the CBGaS has the amino acid substitution L276T or L276P relative to the amino acid sequence of SEQ ID NO: 56.

223. The CBGaS enzyme of any one of embodiments 194-197 and 204-222, wherein the CBGaS has the amino acid substitution A279C or A279S relative to the amino acid sequence of SEQ ID NO: 56.

224. The CBGaS enzyme of any one of embodiments 194-197 and 204-223, wherein the CBGaS has the amino acid substitution A282P relative to the amino acid sequence of SEQ ID NO: 56.

225. The CBGaS enzyme of any one of embodiments 194-197 and 204-224, wherein the CBGaS has the amino acid substitution N309F relative to the amino acid sequence of SEQ ID NO: 56.

226. The CBGaS enzyme of any one of embodiments 194-197 and 204-225, wherein the CBGaS has the amino acid substitution M311L relative to the amino acid sequence of SEQ ID NO: 56.

227. The CBGaS enzyme of any one of embodiments 194-197 and 204-226, wherein the CBGaS has the amino acid substitution S312L relative to the amino acid sequence of SEQ ID NO: 56.

228. The CBGaS enzyme of any one of embodiments 194-197 and 204-227, wherein the CBGaS has the amino acid substitution Y319L relative to the amino acid sequence of SEQ ID NO: 56.

229. The CBGaS enzyme of any one of embodiments 194-197 and 204-228, wherein the CBGaS has the amino acid substitution I324E or I324K relative to the amino acid sequence of SEQ ID NO: 56.

230. The CBGaS enzyme of any one of embodiments 194-197 and 204-229, wherein the CBGaS has the amino acid substitution L325P or L325A relative to the amino acid sequence of SEQ ID NO: 56.

231. A non-naturally occurring CBGaS enzyme having an amino acid sequence that is at least 90% identical (e.g., at least 91%, at least 92%, at least 93%, at least 94%, at least 95%, at least 96%, at least 97%, at least 98%, at least 99%, or 100% identical) to the amino acid sequence of SEQ ID NO: 63.

232. A non-naturally occurring CBGaS enzyme having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 63, wherein the CBGaS has one or more amino acid substitutions relative to the amino acid sequence of SEQ ID NO: 63 selected from I109T, F119L, S245L, S247Y, M270T, C280L, S295D, V314L, A324F, and S361I.

233. The CBGaS enzyme of any one of embodiments 194-196, 231, and 232, wherein the CBGaS has the amino acid substitution I109T relative to the amino acid sequence of SEQ ID NO: 63.

234. The CBGaS enzyme of any one of embodiments 194-196 and 231-233, wherein the CBGaS has the amino acid substitution F119L relative to the amino acid sequence of SEQ ID NO: 63.

235. The CBGaS enzyme of any one of embodiments 194-196 and 231-234, wherein the CBGaS has the amino acid substitution S245L relative to the amino acid sequence of SEQ ID NO: 63.

236. The CBGaS enzyme of any one of embodiments 194-196 and 231-235, wherein the CBGaS has the amino acid substitution S247Y relative to the amino acid sequence of SEQ ID NO: 63.

237. The CBGaS enzyme of any one of embodiments 194-196 and 231-236, wherein the CBGaS has the amino acid substitution M270T relative to the amino acid sequence of SEQ ID NO: 63.

238. The CBGaS enzyme of any one of embodiments 194-196 and 231-237, wherein the CBGaS has the amino acid substitution C280L relative to the amino acid sequence of SEQ ID NO: 63.

239. The CBGaS enzyme of any one of embodiments 194-196 and 231-238, wherein the CBGaS has the amino acid substitution S295D relative to the amino acid sequence of SEQ ID NO: 63.

240. The CBGaS enzyme of any one of embodiments 194-196 and 231-239, wherein the CBGaS has the amino acid substitution V314L relative to the amino acid sequence of SEQ ID NO: 63.

241. The CBGaS enzyme of any one of embodiments 194-196 and 231-240, wherein the CBGaS has the amino acid substitution A324F relative to the amino acid sequence of SEQ ID NO: 63.

242. The CBGaS enzyme of any one of embodiments 194-196 and 231-241, wherein the CBGaS has the amino acid substitution S361I relative to the amino acid sequence of SEQ ID NO: 63.

243. A CBGaS enzyme having an amino acid sequence that is at least 90% identical (e.g., at least 91%, at least 92%, at least 93%, at least 94%, at least 95%, at least 96%, at least 97%, at least 98%, at least 99%, or 100% identical) to the amino acid sequence of SEQ ID NO: 64.

244. A non-naturally occurring CBGaS enzyme having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 64, wherein the CBGaS has one or more amino acid substitutions relative to the amino acid sequence of SEQ ID NO: 64 selected from M275S, M275T, T276C, T276F, K291H, V292Y, V292H, V292F, G310C, F314N, A331C, A331T, and A347I.

245. The CBGaS enzyme of any one of embodiments 194-196, 243, and 244, wherein the CBGaS has the amino acid substitution M275S or M275T relative to the amino acid sequence of SEQ ID NO: 64.

246. The CBGaS enzyme of any one of embodiments 194-196 and 243-245, wherein the CBGaS has the amino acid substitution T276C or T276F relative to the amino acid sequence of SEQ ID NO: 64.

247. The CBGaS enzyme of any one of embodiments 194-196 and 243-246, wherein the CBGaS has the amino acid substitution K291H relative to the amino acid sequence of SEQ ID NO: 64.

248. The CBGaS enzyme of any one of embodiments 194-196 and 243-247, wherein the CBGaS has the amino acid substitution V292Y, V292H, or V292F relative to the amino acid sequence of SEQ ID NO: 64.

249. The CBGaS enzyme of any one of embodiments 194-196 and 243-248, wherein the CBGaS has the amino acid substitution G310C relative to the amino acid sequence of SEQ ID NO: 64.

250. The CBGaS enzyme of any one of embodiments 194-196 and 243-249, wherein the CBGaS has the amino acid substitution F314N relative to the amino acid sequence of SEQ ID NO: 64.

251. The CBGaS enzyme of any one of embodiments 194-196 and 243-250, wherein the CBGaS has the amino acid substitution A331C or A331T relative to the amino acid sequence of SEQ ID NO: 64.

252. The CBGaS enzyme of any one of embodiments 194-196 and 243-251, wherein the CBGaS has the amino acid substitution A347 I relative to the amino acid sequence of SEQ ID NO: 64.

253. An OAC enzyme having an amino acid sequence that is at least 90% identical (e.g., at least 91%, at least 92%, at least 93%, at least 94%, at least 95%, at least 96%, at least 97%, at least 98%, at least 99%, or 100% identical) to the amino acid sequence of any one of SEQ ID NOs: 45-52.

254. A non-naturally occurring OAC enzyme having an amino acid sequence that is at least 90% identical (e.g., at least 91%, at least 92%, at least 93%, at least 94%, at least 95%, at least 96%, at least 97%, at least 98%, or at least 99% identical) to the amino acid sequence of SEQ ID NO: 44, wherein the OAC has one or more amino acid substitutions relative to the amino acid sequence of SEQ ID NO: 44 selected from A2S, L9I, K12S, E14S, F23L, V28L, T47R, Q48R, K49R, S87H, F88Y, and L92Y.

255. The OAC of embodiment 253 or 254, wherein the OAC has the amino acid substitution A2S relative to the amino acid sequence of SEQ ID NO: 44.

256. The OAC of any one of embodiments 253-255, wherein the OAC has the amino acid substitution L91 relative to the amino acid sequence of SEQ ID NO: 44.

257. The OAC of any one of embodiments 253-256, wherein the OAC has the amino acid substitution K12S relative to the amino acid sequence of SEQ ID NO: 44.

258. The OAC of any one of embodiments 253-257, wherein the OAC has the amino acid substitution E14S relative to the amino acid sequence of SEQ ID NO: 44.

259. The OAC of any one of embodiments 253-258, wherein the OAC has the amino acid substitution F23L relative to the amino acid sequence of SEQ ID NO: 44.

260. The OAC of any one of embodiments 253-259, wherein the OAC has the amino acid substitution V28L relative to the amino acid sequence of SEQ ID NO: 44.

261. The OAC of any one of embodiments 253-260, wherein the OAC has the amino acid substitution T47R relative to the amino acid sequence of SEQ ID NO: 44.

262. The OAC of any one of embodiments 253-261, wherein the OAC has the amino acid substitution Q48R relative to the amino acid sequence of SEQ ID NO: 44.

263. The OAC of any one of embodiments 253-262, wherein the OAC has the amino acid substitution K49R relative to the amino acid sequence of SEQ ID NO: 44.

264. The OAC of any one of embodiments 253-263, wherein the OAC has the amino acid substitution S87H relative to the amino acid sequence of SEQ ID NO: 44.

265. The OAC of any one of embodiments 253-264, wherein the OAC has the amino acid substitution F88Y relative to the amino acid sequence of SEQ ID NO: 44.

266. The OAC of any one of embodiments 253-265, wherein the OAC has the amino acid substitution L92Y relative to the amino acid sequence of SEQ ID NO: 44.

267. A nucleic acid encoding the enzyme of any one of embodiments 194-266.

268. A host cell comprising the nucleic acid of embodiment 267.

269. The host cell of embodiment 268, wherein the host cell is a yeast cell or yeast strain.

270. The host cell of embodiment 269, wherein the yeast cell is S. cerevisiae.

SEQUENCE APPENDIX

SEQ ID NO: 1 - AAE from Pseudonocardia sp. N23
MTAAQAPDPAGVPLVERTVPRMLARSAALDPDRPFVVTRERTWSHTDAHRIVATLAAAFTDRGIGQGSR
VAVMMPTSPRHVWLLLALAHLRAVPVALNPDASGEVLRYFVADSECVLGVVDQERAAAFATAAGPDGPP
AIVLPPGADDLGELGSAGPGPLDPGAASFSDTFVVLYTSGSTGMPKATAVTHAQVITCGAVFTDRLGLGP
ADRLYTCLPLFHINATAYSLSGALVSGASLALGPHFSATTFWDDVADLGATEVNAMGSMVRILQSRPPRP
AERAHRVRTMFVAPLPPDAVELSERFGLDFATCYAQTEWLPSSMTRPGEGYGRPGATGPVLPWTEVRI
VGDDDRPLPAGQTGEIILRPRDPYTTFQGYLGKPQETVDAWRNLWFHTGDLGDIGPDGWLHYRGRRKD
VIRRRGENIPATVVEDLLAGHPDIAEVAAVSVPAHISEEEIFAFVVPGAGAALTTADVEAHAHAVLPRYMVP
SYLALVPDLPRTATNKIAKVELTERARAAVEGTGDPADAPTRTSAADRVVVPAAE
SEQ ID NO: 2 - AAE from Pseudomonas putida
MMVPTLEHELAPNEANHVPLSPLSFLKRAAQVYPQRDAVIYGARRYSYRQLHERSRALASALERVGVQP
GERVAILAPNIPEMLEAHYGVPGAGAVLVCINIRLEGRSIAFILRHCAAKVLICDREFGAVANQALAMLDAP
PLLVGIDDDQAERADLAHDLDYEAFLAQGDPARPLSAPQNEWQSIAINYTSGTTGDPKGVVLHHRGAYLN
ACAGALIFQLGPRSVYLWTLPMFHCNGWSHTWAVTLSGGTHVCLRKVQPDAINAAIAEHAVTHLSAAPV
VMSMLIHAEHASAPPVPVSVITGGAAPPSAVIAAMEARGFNITHAYGMTESYGPSTLCLWQPGVDELPLE
ARAQFMSRQGVAHPLLEEATVLDTDTGRPVPADGLTLGELVVRGNTVMKGYLHNPEATRAALANGWLH
TGDLAVLHLDGYVEIKDRAKDIIISGGENISSLEIEEVLYQHPEVVEAAVVARPDSRWGETPHAFVTLRADA
LASGDDLVRWCRERLAHFKAPRHVSLVDLPKTATGKIQKFVLREWARQQEAQIADAEH
SEQ ID NO: 3 - AAE from Streptomyces sp.ADI96-02
MLSTMQDVPLTVTRILQHGMTIHGKSQVTTWTGEPEPHRRTFAEIGARATRLAHALRDELGIDGDQRVAT
LMWNNAEHVEAYLAVPSMGAVLHTLNLRLPAEQLIWIVNHADDKVVIVNGSLLPLLVPLLPHLPTVEHVVV
SGPGDRSALAGVAPRVHEYEELIADRPTTYDWPELDERQAAAMCYTSGTTGDPKGVVYSHRSVYLHSM
QVNMTESMGLTDKDTTLVVVPQFHVNAWGLPHATFMAGVNMLMPDRFLQPAPLADMIERERPTHAAAV
PTIWQGLLAEVTAHPRDLTSMASVTIGGAACPPSLMEAYDKLGVRLCHAWGMTETSPLGTMANPPAGLS
AEEEWPYRVTQGRFPAGVEARLVGPAGDHLPWDGRSAGELEVRGAWIAGAYYGGADGEHLRPEDKFS
ADGWLKTGDVGVISADGFLTLTDRAKDVIKSGGEWISSVELENALMAHPDVAEAAVVAVPDEKWGERPL
ATVVLKEGAEVGYEALKVFLADSGIAKWQLPERWTVIPAVPKTSVGKFDKKVIRKQYADGELDITQL
SEQ ID NO: 4 - AAE from Erythrobacter citreus LAMA 915
MSRAECRDRLTAPGERFEIETIDIRGVPTRVWKHAPTNMRQVAMAARTHGDRLFAIYEDERVTYEAWFR
AVARMAAELRERGVAKGDRVALAMRNLPEWPVAFFAATTIGAICVPLNAWWTGPELAFGLANSGAKLLV
CDAERWERIAPHRGELPDLEHALVSRSDAPLEGAEQLEDLLGTPKDYAALPSAALPQVDIDPEDEATIFYT
SGTTGQPKGALGTHRNLCTNIMSSAYNGAIAFLRRGEEPPAPVQKVGLTVIPLFHVTACSAGLMGYVVAG
HTMVFMHKWDPVKAFQLIEREKVNLTGGVPTIAWQLLEHPERANYDLSSLEAVAYGGAPAAPELVRKIHE
EFGALPANGWGMTETMATVTGHSSEDYLNRPDSCGPPVAVADLKIVGDDGVTELPVGEVGELWARGP
MVVKGYWNRPEATAETFVDGWVRTGDLARLDEEGWCYIVDRAKDMIIRGGENIYSSEVENVLYDHPAVT
DAALVAIAHPTLGEEPAAVVHLAPGMSATEDELREWVAARLAKFKVPVRIAFVQDTLPRNANGKILKKDLG
AFFA
SEQ ID NO: 5 - AAE from Saccharomyces cerevisiae
MVAQYTVPVGKAANEHETAPRRNYQCREKPLVRPPNTKCSTVYEFVLECFQKNKNSNAMGWRDVKEIH
EESKSVMKKVDGKETSVEKKWMYYELSHYHYNSFDQLTDIMHEIGRGLVKIGLKPNDDDKLHLYAATSHK
WMKMFLGAQSQGIPVVTAYDTLGEKGLIHSLVQTGSKAIFTDNSLLPSLIKPVQAAQDVKYIIHFDSISSED
RRQSGKIYQSAHDAINRIKEVRPDIKTFSFDDILKLGKESCNEIDVHPPGKDDLCCIMYTSGSTGEPKGVVL
KHSNVVAGVGGASLNVLKFVGNTDRVICFLPLAHIFELVFELLSFYWGACIGYATVKTLTSSSVRNCQGDL
QEFKPTIMVGVAAVWETVRKGILNQIDNLPFLTKKIFWTAYNTKLNMQRLHIPGGGALGNLVFKKIRTATG
GQLRYLLNGGSPISRDAQEFITNLICPMLIGYGLTETCASTTILDPANFELGVAGDLTGCVTVKLVDVEELG
YFAKNNQGEVWITGANVTPEYYKNEEETSQALTSDGWFKTGDIGEWEANGHLKIIDRKKNLVKTMNGEYI
ALEKLESVYRSNEYVANICVYADQSKTKPVGIIVPNHAPLTKLAKKLGIMEQKDSSINIENYLEDAKLIKAVY
SDLLKTGKDQGLVGIELLAGIVFFDGEWTPQNGFVTSAQKLKRKDILNAVKDKVDAVYSSS
SEQ ID NO: 6 - AAE from Cannabis sativa
MGKNYKSLDSVVASDFIALGITSEVAETLHGRLAEIVCNYGAATPQTWINIANHILSPDLPFSLHQMLFYGC
YKDFGPAPPAWIPDPEKVKSTNLGALLEKRGKEFLGVKYKDPISSFSHFQEFSVRNPEVYWRTVLMDEM
KISFSKDPECILRRDDINNPGGSEWLPGGYLNSAKNCLNVNSNKKLNDTMIVWRDEGNDDLPLNKLTLDQ
LRKRVWLVGYALEEMGLEKGCAIAIDMPMHVDAVVIYLAIVLAGYVVVSIADSFSAPEISTRLRLSKAKAIFT
QDHIIRGKKRIPLYSRVVEAKSPMAIVIPCSGSNIGAELRDGDISWDYFLERAKEFKNCEFTAREQPVDAYT
NILFSSGTTGEPKAIPWTQATPLKAAADGWSHLDIRKGDVIVWPTNLGWMMGPWLVYASLLNGASIALYN
GSPLVSGFAKFVQDAKVTMLGVVPSIVRSWKSTNCVSGYDWSTIRCFSSSGEASNVDEYLWLMGRANY
KPVIEMCGGTEIGGAFSAGSFLQAQSLSSFSSQCMGCTLYILDKNGYPMPKNKPGIGELALGPVMFGASK
TLLNGNHHDVYFKGMPTLNGEVLRRHGDIFELTSNGYYHAHGRADDTMNIGGIKISSIEIERVCNEVDDRV
FETTAIGVPPLGGGPEQLVIFFVLKDSNDTTIDLNQLRLSFNLGLQKKLNPLFKVTRVVPLSSLPRTATNKIM
RRVLRQQFSHFE
SEQ ID NO: 7 - AAE from Citreicella sp. SE45
MSLAADNVLLVEEGRPATAEHPSAGPVYRCKYAKDGLLDLPTDIDSPWQFFSEAVKKYPNEQMLGQRVT
TDSKVGPYTWITYKEAHDAAIRIGSAIRSRGVDPGHCCGIYGANCPEWIIAMEACMSQGITYVPLYDSLGV
NAVEFIINHAEVSLVFVQEKTVSSILSCQKGCSSNLKTIVSFGEVSSTQKEEAKNQCVSLFSWNEFSLMGN
LDEANLPRKRKTDICTIMYTSGTTGEPKGVILNNAAISVQVLSIDKMLEVTDRSCDTSDVFFSYLPLAHCYD
QVMEIYFLSRGSSVGYWRGDIRYLMDDVQALKPTVFCGVPRVYDKLYAGIMQKISASGLIRKKLFDFAYN
YKLGNMRKGFSQEEASPRLDRLMFDKIKEALGGRAHMLLSGAAPLPRHVEEFLRIIPASNLSQGYGLTES
CGGSFTTLAGVFSMVGTVGVPMPTVEARLVSVPEMGYDAFSADVPRGEICLRGNSMFSGYHKRQDLTD
QVLIDGWFHTGDIGEWQEDGSMKIIDRKKNIFKLSQGEYVAVENLENTYSRCPLIAQIWVYGNSFESFLVG
VVVPDRKAIEDWAKLNYQSPNDFESLCQNLKAQKYFLDELNSTAKQYQLKGFEMLKAIHLEPNPFDIERD
LITPTFKLKRPQLLQHYKGIVDQLYSEAKRSMA
SEQ ID NO: 8 - AAE from Bacillus subtilis (strain 168)
MDNLVLCEANNVPLTPITFLKRASECYPNRTSIIYGQTRFTWPQTYDRCCRLAASLLSLNITRNDVVSILAP
NVPAMYEMHFSVPMTGAVLNPINTRLDAKTIAIILRHAEPKILFVDYEFAPLIQEVLRLIPTYQSQPHPRIILIN
EIDSTTKPFSKELDYEGLIRKGEPTPSSSASMFRVHNEHDPISLNYTSGTTADPKGVVISHQGAYLSALSSII
GWEMGIFPVYLWTLPMFHCNGWTHTWSVAARGGTNVCIRHVTAPEIYKNIELHGVTHMSCVPTVFRFLL
EGSRTDQSPKSSPVQVLTGGSSPPAVLIKKVEQLGFHVMHGYGLTEATGPVLFCEWQDEWNKLPEHQQ
IELQQRQGVRNLTLADVDVKNTKTLESVPRDGKTMGEIVIKGSSLMKGYLKNPKATSEAFKHGWLNTGDI
GVIHPDGYVEIKDRSKDIIISGGENISSIEVEKVLYMYQEVLEAAVVAMPHPLWGETPCAFVVLKKGEEGLV
TSEGDLIKYCRENMPHFMCPKKVVFFQELPKNSNGKILKSKLRDIAKALVVREDDAGSKKVHQRSIEHVSS
RL
SEQ ID NO: 9 - AAE from Bhargavaea cecembensis DSE10
MYTDHGWIMKRADITPDGTALIDVHTGQRWTYRELAGRTAAYMEQFRSAGLRKGERVAVLSHNRIDLFA
VLFACAGRGLIYVPMNWRLSESELRYIVSDSGPSLLLHDHEHAGRAAGLGIPAALLDSVPATSVNLRTEQA
AGRLDDPWMMIYTGGTTGRPKGVVLTFESVNWNAINTIISWNLSARDCTLNYMPLFHTGGLNALSLPILM
AGGTVVIGRKFDPEEAIRALNDYRTTISLFVPTMHQAMLDTDLFWESDFPTVDVFLSGGAPCPQTVYDAY
RKKGVRFREGYGMTEAGPNNFIIDPDTAMRKRGAVGKSMQFNEVRILDAKGRPCRAGEVGELHLRGRH
LFSHYWNNEEATQEALKEGWFSTGDLASRDEDGDYFIVGRKKEMIISGGENIYPQEVEQCLIGHDGVREI
AVIGIADRKWGERVVAFIVAQPGNIPKTEELLKHCAQTLGSYKVPKDFFFVQELPITDIGKIDKKQLAIMAEE
LKKEEMQHPGQSG
SEQ ID NO: 10 - AAE from Saccharomyces cerevisiae
MTEQYSVAVGEAANEHETAPRRNIRVKDQPLIRPINSSASTLYEFALECFTKGGKRDGMAWRDIIDIHETK
KTIVKRVDGKDKPIEKTWLYYELTPYITMTYEEMICVMHDIGRGLIKIGVKPNGENKFHIFASTSHKWMKTF
LGCMSQGIPVVTAYDTLGESGLIHSMVETDSVAIFTDNQLLSKLAVPLKTAKNVKFVIHNEPIDPSDKRQN
GKLYKAAKDAVDKIKEVRPDIKIYSFDEIIEIGKKAKDEVELHFPKPEDPACIMYTSGSTGTPKGVVLTHYNI
VAGIGGVGHNVIGWIGPTDRIIAFLPLAHIFELTFEFEAFYWNGILGYANVKTLTPTSTRNCQGDLMEFKPT
VMVGVAAVWETVRKGILAKINELPGWSQTLFWTVYALKERNIPCSGLLSGLIFKRIREATGGNLRFILNGG
SAISIDAQKFLSNLLCPMLIGYGLTEGVANACVLEPEHFDYGIAGDLVGTITAKLVDVEDLGYFAKNNQGEL
LFKGAPICSEYYKNPEETAAAFTDDGWFRTGDIAEWTPKGQVKIIDRKKNLVKTLNGEYIALEKLESIYRSN
PYVQNICVYADENKVKPVGIVVPNLGHLSKLAIELGIMVPGEDVESYIHEKKLQDAVCKDMLSTAKSQGLN
GIELLCGIVFFEEEWTPENGLVTSAQKLKRRDILAAVKPDVERVYKENT
SEQ ID NO: 11 - AAE from Deltaproteobacteria bacterium ADurb.Bin022
MHKFTLDKPDNLVDWWGESVTRFADRPLFGTKNKEGVYKWATYKEIGNRIDNLRAGLTQLGIGKDDVVG
IIANNRPEWAVIGFATWGCLARYVPMYEAELVQVWKYIINDSGAKVLFVSNPAIYEKIKDFPKDIPTLKHIFII
ESDGDNSMASLEKKGAAKPVAPKSPKAEDVAELIYTSGTTGNPKGVLLMHMNFTSNSHAGLKMYPELYE
NEVVSLTILPWAHVFGQTAELFAIIRLGGRMGLIESTKTIINDIVQIKPTFIIAVPTVFNRIYDGLWNKMNKDG
GLARALFVMGVEAAKKKRILAEKGQSDLMTNFKVAVADKIVFKKIRERMGGRMLGSMTGSAAMNVEISKF
FFDIGIPIYDCYGLTETSPGITMNGSQAYRIGSVGRPIDKVKVVIDSSVVEEGATDGEIIAYGPNVMKGYHN
RPEDTKAALTPDGGFRTGDRGRLDKDGYLFITGRIKEQYKLENGKFCFPVSLEENICLASFVQQAVVYGL
NRPYNVCIVVPDFDVLLDYAKEKGLPTDIKTLVEREDIIHMISEAVTGQLKGKFGGYEIPKKFIILPEAFSLDN
GMLTQTMKLKRKVILDKLNDRIEALYKEDK
SEQ ID NO: 12 - AAE from Alcaligenes xylosoxydans (Achromobacter xylosoxidans)
MYSRIHEPHACTLTDALREWAASRPAAPWLEDSQGIAFTVGQAFTSSQRFASFLHHQLGVQPEERVGVF
MSNSCAMVATTFGIGYLRATAVMLNTELRSSFLRHQLNDCQLATIVVDSALVEHVASLADELPHLRTLVVV
GDAPAAVPERWRQVAWMDSSACAPWEGPAPRPEDIFCIMYTSGTTGPSKGVLMPHCHCALLGLGAIRS
LEITEADKYYICLPLFHANGLFMQLGATVLAGIPAFLKQRFSASTWLADIRRSGATLTNHLGTTAMFVINQP
PTEQDRDHRLRASLSAPNPAQHEAVFRERFGVKDVLSGFGMTEVGIPIWGRIGHAAPNAAGWAHEDRF
EICIADPETDVPVLAGQVGEILVRPKVPFGFMAGYLNVPAKTVEAWRNLWFHTGDAGTRDEQGLITFVDRI
KDCIRRRGENISATEVEVVVGQLPGVHEVAAYAVPAQGAGGEDEVMLALVPSEGAALDMADIVRQASAQ
LPRFAKPRYLRQMDSLPKTATGKIQRAVLRQQGSAGAYDAEAAPAR
SEQ ID NO: 13 - AAE from Novosphingobium sp. MD-1
MQFTQGLERAVQHHPDVTATICRARSQTFAELYERVTGLAGCLASRSLAKGARIAVLALNSDHYLEVYLA
TAWAGGVIVPVNFRWSPAEIAYSLNDAGCVALMVDQHHAALVPTLREQCPGLQHIFLMGGTEESDDLPG
LDALIAAAEPLQNAGAGGDDLLGIFYTGGTTGRPKGVMLSHANLCSSGLSMLAEGVFNEGAVGLHVAPM
FHLADMLLTTCLVLRGCTHVMLPAFSPDAVLDHVARFGVTDTLVVPAMLQAIVDHPAIGNFDTSSLCNILY
GASPASETLLRRTMAAFPDVRLTQGYGMTESAAFICALPWHQHVVDNDGPNRLRAAGRSTFDVHLQIVD
PDDRELPRGEIGEIIVKGPNVMQGYYNMPEATAETLRGGWLHTGDMAWMDEEGYVFIVDRAKDMIISGG
ENIYSAEVENAVASHPAVAANAVIGIPHEQMGEAVHVALVLRPGSELSLEALQAHCRALIAGYKVPRSMEV
RPSLPLSGAGKILKTELREPFWKGRDRAVG
SEQ ID NO: 14 - AAE from Thermus thermophilus (strain HB8/ATCC 27634/DSM 579)
MEGERMNAFPSTMMDEELNLWDFLERAAALFGRKEVVSRLHTGEVHRTTYAEVYQRARRLMGGLRALG
VGVGDRVATLGFNHFRHLEAYFAVPGMGAVLHTANPRLSPKEIAYILNHAEDKVLLFDPNLLPLVEAIRGE
LKTVQHFVVMDEKAPEGYLAYEEALGEEADPVRVPERAACGMAYTTGTTGLPKGVVYSHRALVLHSLAA
SLVDGTALSEKDVVLPVVPMFHVNAWCLPYAATLVGAKQVLPGPRLDPASLVELFDGEGVTFTAGVPTV
WLALADYLESTGHRLKTLRRLVVGGSAAPRSLIARFERMGVEVRQGYGLTETSPVVVQNFVKSHLESLSE
EEKLTLKAKTGLPIPLVRLRVADEEGRPVPKDGKALGEVQLKGPWITGGYYGNEEATRSALTPDGFFRTG
DIAVWDEEGYVEIKDRLKDLIKSGGEWISSVDLENALMGHPKVKEAAVVAIPHPKWQERPLAVVVPRGEK
PTPEELNEHLLKAGFAKWQLPDAYVFAEEIPRTSAGKFLKRALREQYKNYYGGA
SEQ ID NO: 15 - AAE from Bradyrhizobium sp. CI-41S
MDWSQHAIPPMRLEPRFGDRVVPAFVDRPASLWAMIADAVAQNGGGEALVCGDIRISWHEVARRAAKV
AAGFAKLGLNSGDRVAILLGNRIEFVLTMFAAAHAGLVTVLLSTRQQKPEIAYVLNDCGARALVHEATLAE
RIPDAADIPGLAHRIAVSDDAASQFAVLLDHPPAPAPAAVSEEDTAMILYTSGTTGRPKGAMLAHCNIIHSS
MVFASTLRLTQADRSIAAVPLAHVTGAVANITTMVRCAGTLIIMPEFKAAEYLKVAARERVSYTVMVPAMY
NLCLLQPDFDSYDLSSWRIGGFGGAPMPVATIERLDAKIPGLKLANCYGATETTSPSTLMPGELTAAHIDS
VGLPCPGAEIIVMGPDGRELPRGEIGELWIRSASVIKGYWNNPKATAESFTDGFWHSGDLGSVDAENFV
RVFDRQKDMINRGGLKIYSAEVESVLAGHPAVIESAIIAKPCPVLGERVHAVIVTRTEVDAESLRAWCAERL
SDYKVPETMTLTTTPLPRNANGKVVKRQLRETLAAGQAPA
SEQ ID NO: 16 - AAE from Bradyrhizobium sp. CI-41S
MAGPAVLTVADTIARSFLLAVQTRGDRPAIREKKFGIWQPTSWREWLQISKDIAHGLHASGFRPGDVASII
ANAVPEWVYADMGILCAGGVSSGIYPTDSTAQVEYLVNDSRTKIVFVEDEEQLDKVLACRARCPTLEKIVV
FDMEGLSGFSDPMVLSFAEFAALGRNHAHGNAALWDEMTGSRTASDLAILVYTSGTTGPPKGAMHSNR
SVTHQMRHANDLFPSTDSEERLVFLPLCHVAERVGGYYISIALGSVMNFAESPETVPDNLREVQPTAFLA
VPRVWEKFYSGITIALKDATPFQNWMYGRALAIGNRMTECRLEGETPPLSLRLANRAAYWLVFRNIRRML
GLDRCRIALTGAAPISPDLIRWYLALGLDMREVYGQTENCGVATIMPTERIKLGSVGKAAPWGEVMICPK
GEILIKGDFLFMGYLNQPERTAETIDAKGWLHTGDVGTIDNEGYVRITDRMKDIIITSGGKNVTPSEIENQLK
FSPYVSDAVVIGDKRPYLTCLIMIDQENVEKFAQDHDIPFTNYASLCRAREIQDLIQREVEAVNTKFARVETI
KKFYLIERQLTPEDEELTPTMKLKRSFVNKRYAAEIDAMYGARAVA
SEQ ID NO: 17 - AAE from Bacillus subtilis (strain 168)
MNLVSKLEETASEKPDSIACRFKDHMMTYQELNEYIQRFADGLQEAGMEKGDHLALLLGNSPDFIIAFFGA
LKAGIVVVPINPLYTPTEIGYMLTNGDVKAIVGVSQLLPLYESMHESLPKVELVILCQTGEAEPEAADPEVR
MKMTTFAKILRPTSAAKQNQEPVPDDTAVILYTSGTTGKPKGAMLTHQNLYSNANDVAGYLGMDERDNV
VCALPMFHVFCLTVCMNAPLMSGATVLIEPQFSPASVFKLVKQQQATIFAGVPTMYNYLFQHENGKKDDF
SSIRLCISGGASMPVALLTAFEEKFGVTILEGYGLSEASPVTCFNPFDRGRKPGSIGTSILHVENKVVDPLG
RELPAHQVGELIVKGPNVMKGYYKMPMETEHALKDGWLYTGDLARRDEDGYFYIVDRKKDMIIVGGYNV
YPREVEEVLYSHPDVKEAVVIGVPDPQSGEAVKGYVVPKRSGVTEEDIMQHCEKHLAKYKRPAAITFLDDI
PKNATGKMLRRALRDILPQ
SEQ ID NO: 18 - AAE from Azoarcus olearius
METVIRDVGRMFAKPVVNVETRGDGSRILRSGIPLPDTYARCVGEWVEKWGKETPDQLFLAERDAVSGE
WRKITWGETRRRVIGIATWLLGQKLSAERPVVILSDNSIEHALLMLAAMHVGVPVSSISPGNSLMSRDHAK
LKGNIELLRPGVIFADPVEKFAPALAAIRELHDGVVIAGRNSQPTAGTVPFAEIEVAPDEAAVMAAFNAITP
DTIAKFLFTSGSVGVPKAVINTQRMMCSNQLAKELVWPFLKENRPVLVEWLPWSHTFGSNHNLNMILRW
GGTIWIDDGKPTPAGLDKTVKNLKEISPTVYFNVPRAYDMLVPLLREDKQLRETFFARLNLIFYAGAALPH
HLWEGLEDLSEQTTGHKVTMVSSWGSTETAPMCTDCHFEAERPGVIGVPVPGTALKLVPSADKLEVRVK
GPNIFPGYWKQPDITAKSFDEEGYYMIGDAVEFLDERFPEKGLLFDGRVGEDFKLLTGTWVHVGSLRVA
GIDAMKPVAQDIVVTGHDRDEIGFLVFPNIPECRTLCPDLPPDADIIDLLLNPAVRQRVRQGMALMKQIGG
GSSTYPSRALLMAEPPSVEAGEITDKGYINQRMVLNRRADLVEYLYQDVVDKTVITVHSAI
SEQ ID NO: 19 - AAE from Microbacterium oxydans
MVRSTYPDVEIPEVSIHDFLFGDLSEAELDTVALVDGMSGATTTYRQLVGQIDLFAGALAARGVGVGTTV
GVLCPNVPAFATVFHGILRAGATATTINSLYTADEIANQLTDAGATWLVTVSPLLPGAQAAAEKLGFDADH
VIVLDGAEGHPSLPALLGEGRQAPDVSFDPSTHLAVLPYSSGTTGRPKGVMLTHRNLVANVSQCQPVLG
VDASDRVLAVLPFFHIYGMTVLLNFALRQRAGLATMPRFDLPEFLRIIAEHRTSWVFVAPPIAVALAKHPIV
DQYDLSAVKVIFSGAAPLDGTLASAVANRLGCIVTQGYGMTETSPAVNLISEARTEIDRSTIGPLVPNTEAR
LVDPDSGEDVVVPAEGASEPGELWVRGPQVMVGYLNRPDATAEMLDADGWLHTGDVATVTHDGIYRIV
DRLKELIKYKGYQVAPAVLEAVLLEHPAIADAAVIGAFDDDGQEVPKAFVVRQPDADLDADAVMAHVTSH
VAPHEKVRQVEFIDVIPKSSSGKILRKDLRAR
SEQ ID NO: 20 - AAE from Aspergillus niger (strain CBS 513.88/FGSC A1513)
MLFSQQPLHLTRADELRQSPPKGTPYSVALPGTEKPGRSKVYRAWNATEGVLKSLDPQILTAHDIFESTA
NRLPKNHCLGWRPYNPTTKTYGVYQWLDYQTVQKRRAAFGAGLVELHHKHECSRPGQYGIGLWCQNR
PEWQITDLACMSQSLYSVSIYDVLAPDATEYIINHAELACVVTSLPHIPTLLRLKPQLPNLKIIVSLDPLDGGE
EAGHSKRALLESMAAGQDVSIYTMSQVEELGASVDRPCKPPAPSDTITINYTSGTTGPPKGVVLAHENAV
ASASGALINSIQKAGDTIISYLPLAHIYARMSEHAAFWAGARIGYFHGNILELVDDLKLLKPTGFISVPRLYTR
FGNAIRASTVEAPGFRGALSRHIVATKTANLKNPDPSQATGKHALYDRIWAKKVAAAIGLERSRMLASGS
APLDPSLHQFLRIALGVDVVQGYGLTETYAMACVQSLADLTAGHCGGLIPSTEACLMSLPDMEYSVDDKP
YPRGELMLRGANVFREYFKDPEETAKAVTEDGWFRTGDVCKIDEMGRIVIIDRRKNVLKLAQGEYISPERL
EGVYMSEMGYLAQGYVHGDSVQTFLVAIFGVQPDTFAVFASKVLGRTIEATDIEGIRSVLNDPKIRKAVLK
DLNRIAKKHKLAGYERIKNCALMIDPFTIENNLLTPTLKLKRPPTTKKYRQVLDELYAEALAEESAPKAKL
SEQ ID NO: 21 - AAE from Brevibacterium yomogidense
MSWFDERPWLRTLGLTETEAVPLEPSTPLRDLADTVAAHPTTAAWTHYGQSATYAEFDRQTTAFAAYLA
ESGIRPGDAVAVYAQNSPHFPIATYGIWKAGAVVVPLNPMYRDELTHAFADADVKAIVVQKALYLMRVKE
YAADLPLVVLAGDLDWAQDGPDAVFGAYADLPDVPLPDLRTVVDERLDTDFEPLTVRPEDPALIGYTSGT
SGKAKGALHPHSSISSNSRMAARNAGLPQGAGVVSLAPLFHITGFICQMIASTANGSTLVLNHRFDPASFL
DLLRQEKPAFMAGPATVYTAMMASPSFGADAFDSFHSIMSGGAPLPEGLVKRFEEKTGHYIGQGYGLTE
TAAQAVTVPHSLRAPVDPESGNLSTGLPQRDAMVRILDDDGNPVGPREVGEVAISGPMVATEYLGNPQA
TADSLPGGELRTGDVGFMDPDGWVFIVDRKKDMINASGFKVWPREVEDILYMHPAVREGAVVGVPDEY
RGETVVAFVSLQPDSQATAEDIIAHCKEHLASYKAPVEVTIVDELPKTSSGKILRRTVRDEATQARQAQPD
AH
SEQ ID NO: 22 - AAE from Brevibacterium linens
MINNWLAVGLLVVSGILAFNWKRKHPYGQTVEIGEKPENGGRIRRNSACADHLISFLEDDEIYTLYDSLVK
SCKKYGERKCFGERKKDSNGNLGKFEWISYNTYLERCEYIQQGLCELGLKPKSKVGIFSKNRLEWLIVHS
ASFIQSYCVVSFYETLGVESLSYVTEHAEIGLAFCSAETLQKTLDIAKGVKVLKTIICFDSIDKEHYNIAKELG
VTLYTYDEIMKKGKEANGKHKHTPPTPDTLSTIMYTSGTTGPPKGVMITHKNLTSVVCAVSDFIKVYDTDV
HYSYLPYAHVLERVVILAAFHFGAAIGIFSGDISNILVEVKLLSPTLFIGVPRVFERIKTNVFKEISKKPALLRT
LFNGAYNLKYLSIQHGFKLPIIEKVLDLVFFSKIKQALGGKVRVILSGSAPLSFDTEVFLRVVMCCCVLQGY
GASEGCGGDACKRLDDESVGTIGPPFASNEIKLVDVPELGYDSNGEVQTGEVCLRGPSISSGYYKDEEK
TREEFKDGWFHTGDIGRWNRDGSLSIVDRKKNIFKLSQGEYVAVEKIETIVVKSEYVEQVCIYGDSQKSCV
IAIIHPHPESCSEWAGSKKTDKDIKEICKNQDFIKVVLDDIIKNCKKSGLHGFEIPKAIHLTPEAFSDQNNLLT
PSFKLKRHEIKKYFEDEIKKLYSKLD
SEQ ID NO: 23 - AAE from Nocardioides simplex (Arthrobacter simplex)
MSFRYYRDLHPTFADRTEWALPTVLRHHAAERPDAVWLDCPEEGRTWTFAETLTAAERVGRSLLAAGA
EPGDRVVLVAQNSSAFVRTWLGTAVAGLVEVPVNTAYEHDFLAHQVSTVEATLAVVDDVYAARFVAIAEA
AKSIRKFWVIDTGSRDQALATLRDAGWEAAPFEELDEAATAPEVVDATLALPDVRPQDLASVLFTSGTTG
PSKGVAMPHAQMYFFADECVSLVRLTPDDAWMSVTPLFHGNAQFMAAYPTLVAGARFVTRSRFSASRW
VDQLRESRVTVTNFIGVMMDFIWKQDRRDDDADNPLRVVFAAPTAATLVGPMSERYGIEAFVEVFGLTET
SAPIISPYGVDRPAGAAGLAADEWFDVRLVDPETDEEVGVGEIGELVVRPKVPFICSMGYFNMPDKTVEA
WRNLWFHTGDALRRDEDGWFYFVDRFKDALRRRGENISSYEIETSILAHPAVVECAVIAVPASSEAGEDE
VMAYVITGGDAPVPTPAELWAHCDGRIPSFAVPRYLRFVDEMPKTPSQRVQKAKLRALGVTPDTHDREA
SEQ ID NO: 24 - AAE from Pseudomonas putida (Arthrobacter siderocapsulatus)
MNLGKIITRSARYWPDHTAVADSQTRLTYAQLERRSNRLASGLGALGVATGEHVAILAANRVELVEAEVA
LYKAAMVKVPINARLSLDEVVRVLEDSCSVALITDATFAQALAERRAALPMLRQVIALEGEGGDLGYAALL
ERGSEAPCSLDPADDALAVLHYTSGSSGVLKAAMLSFGNRKALVRKSIASPTRRSGPDDVMAHVGPITH
ASGMQIMPLLAVGACNLLLDRYDDRLLLEAIERERVTRLFLVPAMINRLVNYPDVERFDLSSLKLVMYGAA
PMAPALVKKAIELFGPILVQGYGAGETCSLVTVLTEQDHLIEDGNYQRLASCGRCYFETDLRVVNEAFEDV
APGEIGEIVVKGPDIMQGYWRAPALTAEVMRDGYYLTGDLATVDAQGYVFIVDRKKEMIISGGFNVYPSE
VEQVIYGFPEVFEAAVVGVPDEQWGEAVRAVVVLKPGAQLDAAELIERCGRALAGFKKPRGVDFVTELP
KNPNGKVVRRLVREAYWQHSDRRI
SEQ ID NO: 25 - TKS from Dendrobium catenatum
MPSLESIRKAPRANGFASILAIGRANPENFIEQSTYPDFFFRITNSEHLVDLKKKFQRICDKTAIRKRHFVW
NEEFITTNPCLHTFMDKSLDVRQEVAIREIPKLGAKAAAKAIQEWGQPKSRITHLIFCTTSGMDLPGADYQL
TQILGLNPNVERVMLYQQGCFAGGTTLRLAKCLAESRKGARVLVVCAETTTVLFRGPSEEHQEDLVTQAL
FADGASALIVGADPDEAAHERASFVIVSTSQVLLPDSAGAIGGHVSEGGLLATLHRDVPKIVSKNVEKCLE
EAFTPFGITDWNSIFWVPHPGGRAILDLVEERVGLKPEKLLVSRHVLAEYGNMSSVCVHFALDEMRKRSA
IEGKATTGEGLEWGVVFGFGPGLTVETVVLRSVPL
SEQ ID NO: 26 - TKS from Cannabis sativa
MNHLRAEGPASVLAIGTANPENILLQDEFPDYYFRVTKSEHMTQLKEKFRKICDKSMIRKRNCFLNEEHLK
QNPRLVEHEMQTLDARQDMLVVEVPKLGKDACAKAIKEWGQPKSKITHLIFTSASTTDMPGADYHCAKLL
GLSPSVKRVMMYQLGCYGGGTVLRIAKDIAENNKGARVLAVCCDIMACLFRGPSESDLELLVGQAIFGDG
AAAVIVGAEPDESVGERPIFELVSTGQTILPNSEGTIGGHIREAGLIFDLHKDVPMLISNNIEKCLIEAFTPIGI
SDWNSIFWITHPGGKAILDKVEEKLHLKSDKFVDSRHVLSEHGNMSSSTVLFVMDELRKRSLEEGKSTTG
DGFEWGVLFGFGPGLTVERVVVRSVPIKY
SEQ ID NO: 27 - TKS from Arachis hypogaea
MVSVSGIRKVQRAEGPATVLAIGTANPPNCIDQSTYADYYFRVTNSEHMTDLKKKFQRICERTQIKNRHM
YLTEEILKENPNMCAYKAPSLDAREDMMIREVPRVGKEAATKAIKEWGQPMSKITHLIFCTTSGVALPGVD
YELIVLLGLDPCVKRYMMYHQGCFAGGTVLRLAKDLAENNKDARVLIVCSENTAVTFRGPSETDMDSLVG
QALFADGAAAIIIGSDPVPEVEKPIFELVSTDQKLVPGSHGAIGGLLREVGLTFYLNKSVPDIISQNINDALNK
AFDPLGISDYNSIFWIAHPGGRAILDQVEQKVNLKPEKMKATRDVLSNYGNMSSACVFFIMDLMRKRSLE
EGLKTTGEGLDWGVLFGFGPGLTIETVVLRSVAI
SEQ ID NO: 28 - TKS from Dictyostelium discoideum AX4
MNNSNVKSSPSIVKEEIVTLDKDQQPLLLKEHQHIIISPDIRINKPKRESLIRTPILNKFNQITESIITPSTPSLS
QSDVLKTPPIKSLNNTKNSSLINTPPIQSVQQHQKQQQKVQVIQQQQQPLSRLSYKSNNNSFVLGIGISVP
GEPISQQSLKDSISNDFSDKAETNEKVKRIFEQSQIKTRHLVRDYTKPENSIKFRHLETITDVNNQFKKVVP
DLAQQACLRALKDWGGDKGDITHIVSVTSTGIIIPDVNFKLIDLLGLNKDVERVSLNLMGCLAGLSSLRTAA
SLAKASPRNRILVVCTEVCSLHFSNTDGGDQMVASSIFADGSAAYIIGCNPRIEETPLYEVMCSINRSFPNT
ENAMVWDLEKEGWNLGLDASIPIVIGSGIEAFVDTLLDKAKLQTSTAISAKDCEFLIHTGGKSILMNIENSLG
IDPKQTKNTWDVYHAYGNMSSASVIFVMDHARKSKSLPTYSISLAFGPGLAFEGCFLKNVV
SEQ ID NO: 29 - TKS from Spinacia oleracea
MASVDISEIHNVERAKGQANVLAIGTANPPNVMYQADYPDFYFRLTNSEHMTDLKAKFKRICEKTTIKKRY
MHISEDILKEKPDLCDYNASSLDIRQVILAKEVPKVGKDAAMKAIEEWGQAMSKITHLIFCTTSGVDIPGAD
YQLTMLLGLNPSVKRYMLCQQGCHAGGTVLRLAKDLAENNYGSRVLVVCSENTTVCFRGPTETHPDSM
VAQALFADGAGAVIVGAYPDESLNERPIFQIVSTAQTILPNSQGAIEGHLRQIGLAIQLLPNVPDLISNNIDKC
LVEAFNPIGINDWNSIFWIAHPGGPAILGQVESKLGLQESKLTTTWHVLREFGNMSSACVFFIMDETRKRS
LKEGKTTTGDGFDWGVLFGFGPGLTVETVVLRSFPLNQ
SEQ ID NO: 30 - TKS from Chenopodium quinoa
MASVQEIRNAQRADGPATILAIGTANPPNEMYQAEYPDFYFRVTESEHMTDLKKKFKRMCERSMIKKRY
MHVTEELLKENPHMCDYNASSLNTRQDILATEVPKLGKEAAIKAIKEWGQPRSKITHVIFCTTSGVDMPGA
DYQLTKLLGLRPSVKRFMLYQQGCYAGGTVLRLAKDIAENNRGARVLVVCAEITVICFRGPTETHLDSMIG
QALFGDGAGAVIVGADVDESIERPIFQLVWAAQTILPDSEGAIDGHLREVGLAFHLLKDVPGLISKNIEKAL
VEAFKPIGIDDWNSIFWVAHPGGPAILDQVESKLELKQDKLRDTRHVLSEFGNMSSACVLFILDEMRNRSL
KEGKTTTGEGLDWGVLFGFGPGLTVETVMLHSVPITN
SEQ ID NO: 31 - TKS from Cannabis sativa
MASISVDQIRKAQRANGPATVLAIGTANPPTSFYQADYPDFYFRVTKNQHMTELKDKFKRICEKTTIKKRH
LYLTEDRLNQHPNLLEYMAPSLNTRQDMLVVEIPKLGKEAAMKAIKEWGQPKSRITHLIFCSTNGVDMPG
ADYECAKLLGLSSSVKRVMLYQQGCHAGGSVLRIAKDLAENNKGARILTINSEITIGIFHSPDETYFDGMV
GQALFGDGASATIVGADPDKEIGERPVFEMVSAAQEFIPNSDGAVDGHLTEAGLVYHIHKDVPGLISKNIE
KSLVEALNPIGISDWNSLFWIVHPGGPAILNAVEAKLHLKKEKMADTRHVLSEYGNMSSVSIFFIMDKLRKR
SLEEGKSTTGDGFEWGVLFGFGPGLTVETIVLHSLAN
SEQ ID NO: 32 - TKS from Plumbago indica
MAPAVQSQSHGGAYRSNGERSKGPATVLAIATAVPPNVYYQDEYADFFFRVTNSEHKTAIKEKFNRVCG
TSMIKKRHMYFTEKMLNQNKNMCTWDDKSLNARQDMVIPAVPELGKEAALKAIEEWGKPLSNITHLIFCT
TAGNDAPGADFRLTQLLGLNPSVNRYMIYQQGCFAGATALRIAKDLAENNKGARVLIVCCEIFAFAFRGPH
EDHMDSLICQLLFGDGAAAVIVGGDPDETENALFELEWANSTIIPQSEEAITLRMREEGLMIGLSKEIPRLL
GEQIEDILVEAFTPLGITDWSSLFWIAHPGGKAILEALEKKIGVEGKLWASWHVLKEYGNLTSACVLFAMD
EMRKRSIKEGKATTGDGHEYGVLFGVGPGLTVETVVLKSVPLN
SEQ ID NO: 33 - TKS from Ziziphus jujuba
MVTVDEIREAQRAKGPATIMAIGTATPPNAIDQSTFTDYYFRITNSDHKTDLKKKFKTICDKSMIKKRYLYLT
EEHLKQNPNMSEYMAPSLDVRQEIVIAEVPKLGKEAANKAIKEWGQPKSKITHLVFSTISGVDAPGADYQL
TKLLGLNPSVKRIMVYQQGCFAGGTSLRLAKDLAENNKGARVLVVCTEISAINFRGPSETYFDSNVGQILF
GDGASAVVVGSDPLVGVEKPLFELVSASQTIIPDSEGNIEGHICEVGLTIRLSKKVPSLISNNIEKSLVEAFN
PLGISDWNSIFWIAHPGGPAILDQIELKLGLKPEKLRASRHVLSEYGNMSSATVLFILDEMRKKSIEDGLKT
PGEGLEWGVLFGFGPGLTVETVVLHSVTA
SEQ ID NO: 34 - TKS from Anoectochilus roxburghii
MPSLESIRKAPRADGLASILAIGRANPDNFMEQSSFPDFFFRITGSDHLVDLKKKFQRICDRTAIRKRHFV
WNEEFIKANPCFSTFMDNSLNVRQEVAIREIPKLGAEAATKAIKEWGQPKSRITHLIFCTTSGMDLPGADY
QLTRILGLNPNVERVMLYQQGCFAGGTTLRLAKCLAESRKGARVLVVCAETTTVLFRAPSEEHQEDLVTQ
ALFADGASAVIVGADPDEEAHEKASFVIFSTSQVLLPDSEGAIGGHVSEGGLLATLHRDVPQLVSKNVGK
CLEEAFTPLGISDWNSIFWVPHPGGRAILDQIEERVGLKPEKLTTSRHVLAEYGNMSSVCVHFVLDEMRK
KSSKEGKATTGEGLEWGVLFGFGPGLTVETVVLRSVPL
SEQ ID NO: 35 - TKS from Cymbidium hybrid cultivar
MPSLESVKKSNRADGFASILAIGRANPENFIEQSTYPDFFFRVTNSEHLVNLKKKFQRICDKTAIRKRHFV
WNEELLNANPCLGTFMDNSLNVRQEFAIREIPKLGAEAATKAIQEWGQPKSRITHLIFCTTSGMDLPGADY
QLTQILGLNPNIERVMLYQQGCFAGGTTLRLAKCLAESRKGARVLVVCAETTAVLFRAPSEEHQDDLVTQ
ALFADGASALIVGADPDETAHERASFVIVSTSQVLLPDSAGAIGGHVSEGGLIATLHRDVPQIVSKNVGKCL
EEAFTPLGISDWNSIFWVPHPGGRAILDQVEERVGLKPEKLIVSRHVLAEYGNMSSVCVHFALDEMRKRS
KKEGKATTGEGLDWGVLFGFGPGLTVETVVLHSVPI
SEQ ID NO: 36 - TKS from Phalaenopsis equestris
MPSLDSIKKAPRADGFASILAIGRANPDNIIEQSAYPDFYFRVTNSEHLVDLKKKFQRICEKTAIRKRHFVW
NEEFLTSNPCFSTFMDKSLNVRQEVAIREIPKLGAKAATKAIEDWGQPKSRITHLIFCTTSGMDLPGADYQ
LTQILGLNPNVERVMLYQQGCFAGGTTLRLAKCLAESRKGARVLVVCAETTTVLFRAPSEEHQDDLVTQA
LFADGASAVIVGADPDEAADERASFVIVSTSQVLLPDSAGAIGGHVSEGGLLATLHRDVPQIVSKNVGKCL
EEAFTPFGISDWNSIFWVPHPGGRAILDQVEERVGLKPEKLSVSRHVLAEYGNMSSVCVHFALDEMRKR
SANEGKATTGEGLEWGVLFGFGPGLTVETVVLRSVPL
SEQ ID NO: 37 - TKS from Dendrobium catenatum
MPSLESIRKAPRANGFASILAIGRANPENFIEQSTYPDFFFRITNSEHLVDLKKKFQRICDKTAIRKRHFVW
NEEFITTNPCLHTFMDKSLDVRQEVAIREIPKLGAKAAAKAIQEWGQPKSRITHLIFCTTSGMDLPGADYQL
TQILGLNPNVERVMLYQQGCFAGGTTLRLAKCLAESRKGARVLVVCAETTTVLFRGPSEEHQDDLVTQAL
FADGASALIVGADPDEAAHERASFVIVSTSQVLLPDSAGAIGGHVSEGGLLATLHRDVPKIVSKNVEKCLE
EAFTPFGITDWNTIFWVPHPGGRAILDQVEERMGLKPEKLLVSRHVLAEYGNMSSVCVHFALDEMRKRS
AIEGKATTGEGLEWGVLFGFGPGLTVETVVLRSVHL
SEQ ID NO: 38 - TKS from Oncidium hybrid cultivar
MPSLESTKKAPRSHGFASILAIGRANPENFVEQNAYPDLFFRATNSKHLVNLKKKFQRICDKTAIRKRHFA
WNEEFITANPCLQTFMDNSLNVRQEFAITYIPKLGAEAATKAIQEWGQPKSRITHLIFCTTSGMDLPGADY
QLTQILGLNPNVERVMLYQQGCFAGGTTLRLAKCLAESRKGARVLVVCAETTAVLFRAPSEEHQDDLVT
QALFADGASALIVGADPDEAANERASFIIVSTSQVLLPDSAGAIGGHVSEGGLLATLHRDVPQIVSKNVGK
CLEEAFTPLGISDWNSIFWVPHPGGRAILDLVEERVGLKPEKLLVSRHVLAEYGNMSSVCVHFALDEMRR
RSAKEGKATTGEGLDWGVLFGFGPGLTVETVVLHSVPI
SEQ ID NO: 39 - TKS from Apostasia shenzhenica
MPGVEAVAQNISPARSDGLAAILAIGRANPPNIVEQSSFADLYFRLHNSEHLVDLKKKLQRICDRTAIRKRH
FVWDEELLMANPCLRTVTEPSLNARQKVAITEIPKLGAAAATNAIAEWGRPKSDITHLIFCTTSGMDLPGA
DYQLIRLLGLNDNIQRIMLYQQGCFAGGTVLRLAKVLAESRRSARVLIVCAETTTVLVRSPSVENQDDLVT
QALFADGASALIVGADPNAGEKPVFSVFSTSQVLLPDSDGAIGGHVGENGLTATLHRDVPAVISKNVGKC
LEEAFTPLGISDWNSIFWAAHPGGRAILDQVEERVGLKPEKMWASRHVLAEYGNMSSVSVHFALDEIRR
RSAKEGKATTGDGFEWGVLFGFGPGLTVETVVLRSAPISA
SEQ ID NO: 40 - TKS from Paphiopedilum hangianum
MPGLENRKKVEALIRAEGLATIMAIGRANPPNAMEQSTFPDFYFRVTNSEHLVGLKKKFQRICEKTAIRRR
HFVWNEEILNANPCLRTHMEPSLNVRQKIAVAEIPKLGAEAASRAIEEWGQPKSRITHLIFCTTSGMDLPG
ADYKLTRILGLNPNVQRVMLYQQGCFAGGTVLRLAKCFAESRKGARVLVVCSETTTVLVRAPSEDYQDD
LVTQALFADGASALIVGADPDEEAKERPIFTIVSTTQVILPDSDGAIGGHLGEGGLTATLHRDVPLIISKNVS
KCLEEAFAPLGISDWNSIFWAPHPGGRAILDQVEERVGLKPEKLWASRHVLAEYGNMSSVCVHFVLDEIR
KRSAKESKATTGEGFDWGVLFGFGPGLTVETVILRSVPLN
SEQ ID NO: 41 - TKS from Apostasia shenzhenica
MPGLQIISKASSRAADGLAAILAIGRANPPNSMDQSSYPEFYFRVMDSDHLVDLKKKFQRICERTAIRKRH
FVWNEELLRDNPCLRTFMDSSLNVRQKVAVAEIPKLGAAAAERAIEEWGQPRSGITHLIFCTTSGMDLPG
ADYQLTKILGLNADVQRVMLYQQGCFAGGTVLRLAKVLAESRKGARVLVVCAETTTVLIRAPSVEHQDDL
VTQALFADGASALIVGADPVEEVNERPLFSIISASQVILPDSDGAIGGHLGEGGLTATLHRDVPLIISKNVSK
CLEDAFSPLGISDWNSIFWAPHPGGRAILDQVEERVGLKPEKMWASRHVLAEYGNMSSVCVHFVLDEM
RKRSAKEGKPTTGEGLEWGVLFGFGPGLTVETVVLRSHPIN
SEQ ID NO: 42 - TKS from Phalaenopsis equestris
MPNMESIKKEDGLATIMAIGRALPPNSIDQNSFPDFYFRVHNSEHLMDLKNKFRRICERTAIRKRHFVWNE
EVLKQNPCLRTFMEPSLNTRQEIVCSEIPKLGAEAARNAIREWGQPERSITHLIFCTTSGMNLPGADFEAA
QILGLNHSVERVMLYQQGCFAGGTVLRLAKCLAESRRGARVLVICAESTTSLVRSPSREHQYDLIAQALF
ADGASALIIGTEPNAEAGERPIFSIFSTAQVTLPDSGDAIRGYLKEGGLIATLAKDVPLIISENIERCLQEAFG
PLGISDWNSIFWAPHPGGRAILDGIEDKLGLKPEKLWAARHVLAEYGNMSSVCVHYILDEMRRRDVKNGK
APTGDGPEWGVLFGFGPGLTVETVVLRRLFL
SEQ ID NO: 43 - TKS from Bromheadia finlaysoniana
MASQVSPPSINMAPKADGFASILAIGRANPKNFIEQSTFPDFFFRVTNTEHMVDLKKKFQRICDKTSIRKRH
FIWNEELLTANPSLCTFMGNSLNLRHEVAVREIPKLGAEAATKAIQEWGQPKSFITHLVFCTTSGMDLPGA
DYQLTQILGLNLDIERVMLHQQGCFLGGTTLRLAKYLAESRKGARVLVVCAETTTEFFRAPSEEHQEDLVT
QSLFGDGASALIVGADPHEGARERASFILVSSSQVLLANSAHAITGHVSEGGIKATLHRDVPQIISNNLGKC
LEEAFTPLGISDWNSIFWVLHPGGRAILDQVEEKMGLEPEKLLISRHVLLEYGNMSSVCVHFALDEMRKR
SSNEGKATTGEGLEWGVLFGFGPGLTIETVVLRSVSIS
SEQ ID NO: 44 - OAC from Cannabis sativa
MAVKHLIVLKFKDEITEAQKEEFFKTYVNLVNIIPAMKDVYWGKDVTQKNKEEGYTHIVEVTFESVETIQDYI
IHPAHVGFGDVYRSFWEKLLIFDYTPRK
SEQ ID NO: 45 - OAC (synthetic)
MAVKHLIVIKFSDSITEAQKEEFFKTYLNLVNIIPAMKDVYWGKDVTRRNKEEGYTHIVEVTFESVETIQDYII
HPAHVGFGDVYRHYWEKLLIFDYTPRK
SEQ ID NO: 46 - OAC (synthetic)
MSVKHLIVIKFSDEITEAQKEELFKTYVNLVNIIPAMKDVYWGKDVRQRNKEEGYTHIVEVTFESVETIQDYI
IHPAHVGFGDVYRHYWEKLLIFDYTPRK
SEQ ID NO: 47 - OAC (synthetic)
MSVKHLIVIKFSDEITEAQKEELFKTYVNLVNIIPAMKDVYWGKDVTRRNKEEGYTHIVEVTFESVETIQDYII
HPAHVGFGDVYRHYWEKLLIFDYTPRK
SEQ ID NO: 48 - OAC (synthetic)
MSVKHLIVIKFKDSITEAQKEELFKTYVNLVNIIPAMKDVYWGKDVTRRNKEEGYTHIVEVTFESVETIQDYII
HPAHVGFGDVYRHYWEKLLIFDYTPRK
SEQ ID NO: 49 - OAC (synthetic)
MSVKHLIVIKFKDSITEAQKEELFKTYLNLVNIIPAMKDVYWGKDVTRRNKEEGYTHIVEVTFESVETIQDYII
HPAHVGFGDVYRSYWEKLLIFDYTPRK
SEQ ID NO: 50 - OAC (synthetic)
MAVKHLIVIKFSDSITEAQKEELFKTYVNLVNIIPAMKDVYWGKDVTRRNKEEGYTHIVEVTFESVETIQDYII
HPAHVGFGDVYRHYWEKLLIFDYTPRK
SEQ ID NO: 51 - OAC (synthetic)
MSVKHLIVIKFSDSITEAQKEEFFKTYVNLVNIIPAMKDVYWGKDVTRRNKEEGYTHIVEVTFESVETIQDYII
HPAHVGFGDVYRHYWEKLLIFDYTPRK
SEQ ID NO: 52 - OAC (synthetic)
MSVKHLIVIKFKDSITEAQKEELFKTYVNLVNIIPAMKDVYWGKDVRRRNKEEGYTHIVEVTFESVETIQDYII
HPAHVGFGDVYRSYWEKLLIFDYTPRK
SEQ ID NO: 53 - CBGaS from Cannabis sativa
MSIIIFMGLSLVCTFSFQTNYHTLLNPHNKNPKNSLLSYQHPKTPIIKSSYDNFPSKYCLTKNFHLLGLNSHN
RISSQSRSIRAGSDQIEGSPHHESDNSIATKILNFGHTCWKLQRPYVVKGMISIACGLFGRELFNNRHLFS
WGLMWKAFFALVPILSFNFFAAIMNQIYDVDIDRINKPDLPLVSGEMSIETAWILSIIVALTGLIVTIKLKSAPL
FVFIYIFGIFAGFAYSVPPIRWKQYPFTNFLITISSHVGLAFTSYSATTSALGLPFVWRPAFSFIIAFMTVMGM
TIAFAKDISDIEGDAKYGVSTVATKLGARNMTFVVSGVLLLNYLVSISIGIIWPQVFKSNIMILSHAILAFCLIF
QTRELALANYASAPSRQFFEFIWLLYYAEYFVYVFI
SEQ ID NO: 54 - CBGaS from Cannabis sativa
MAGSDQIEGSPHHESDNSIATKILNFGHTCWKLQRPYVVKGMISIACGLFGRELFNNRHLFSWGLMWKA
FFALVPILSFNFFAAIMNQIYDVDIDRINKPDLPLVSGEMSIETAWILSIIVALTGLIVTIKLKSAPLFVFIYIFGIF
AGFAYSVPPIRWKQYPFTNFLITISSHVGLAFTSYSATTSALGLPFVWRPAFSFIIAFMTVMGMTIAFAKDIS
DIEGDAKYGVSTVATKLGARNMTFVVSGVLLLNYLVSISIGIIWPQVFKSNIMILSHAILAFCLIFQTRELALA
NYASAPSRQFFEFIWLLYYAEYFVYVFI
SEQ ID NO: 55 - CBGaS from Stachybotrys bisbyi
MPATRTPIHPEAAAYKNPRYQSGPLSVIPKSFVPYCELMRLELPHGNFLGYFPHLVGLLYGSSASPARLP
ANEVAFQAVLYIGWTFFMRGAGCAWNDVVDQDFDRKTTRCRVRPVARGAVSTTSANIFGFAMVALAFA
CISPLPAECQRLGLMTTVLSIIYPFCKRVTNFAQVILGMTLAINFILAAYGAGLPAIEAPYTVPTICVTTAITLL
VVFYDVVYARQDTADDLKSGVKGMAVLFRNYVEILLTSITLVIAGLIATTGVLVDNGPYFFVFSVAGLLAALL
AMIGGIRYRIFHTWNSYSGWFYALAIFNLLGGYLIEYLDQVPMLNKA
SEQ ID NO: 56 - CBGaS from Stachybotrys chartarum IBT 40288
MSAKVSPMAYTNPRYETGPLSLIPKPIVPYFELMRFELPHGYYLGYFPHLVGIMYGASAGPERLPARDLVF
QALLYVGWTFAMRGAGCAWNDNIDQDFDRKTERCRTRPIARGAVSTTAGHVFAVAGVALAFLCLSPLPT
ECHQLGVLVTVLSVIYPFCKRFTNFAQVILGMTLAANFILAAYGAGLPALEQPYTRPTMSATLAITLLVVFYD
VVYARQDTADDLKSGVKGMAVLFRNHIEVLLAVLTCTIGGLLAATGVSVGNGPYYFLFSVAGLTVALLAMI
GGIRYRIFHTWNGYSGWFYVLAIINLMSGYFIEYLDNAPILARGS
SEQ ID NO: 57 - CBGaS from Stachybotrys chlorohalonata (strain IBT 40285)
MSPKVSSMPYTNPRYESGPLSLIPKSIVPYFELMRFELPHGYYLGYFPHLVGIMYGASAGPERLPARDLVF
QALLYVGWTFAMRGAGCAWNDNIDQDFDRKTERCRTRPIARGAVSTTAGHIFAVAGVALAFLCLSPLPTE
CHQLGVLVTVLSVIYPFCKRFTNFAQVILGMTLAANFILAAYGAGLPALEQPYTRPTMFATLAITLLVVFYDV
VYARQDTADDLKSGVKGMAVLFRNHIEVLLAVLTCTIGGLLAATGVSVGNGPYYFLFSVAGLTVALLAMIG
GIRYRIFHTWNGYSGWFYVLAIINLMSGYFIEYLDNAPILARGS
SEQ ID NO: 58 - CBGaS from Stachybotrys chartarum (strain CBS 109288/IBT 7711)
MSAKVSPMAYTNPRYERGPLSLIPKPIVPYFELMRFELPHGYYLGYFPHLVGIMYGASAGPERLPARDLV
FQALLYVGWTFAMRGAGCAWNDNIDQDFDRKTERCRTRPIARGAVSTTAGHVFAVAGVALAFLCLSPLP
TECHQLGVLVTVLSVIYPFCKRFTNFAQVILGMTLAANFILAAYGAGLPALEQPYTRPTMSATLAITLLVVFY
DVVYARQDTADDLKSGVKGMAVLFRNHIEVLLAVLTCTIGGLLAATGVSVGNGPYYFLFSVAGLTVALLAM
IGGIRYRIFHTWNGYSGWFYVLAIINLMSGYFIEYLDNAPILARGS
SEQ ID NO: 59 - Parent for Chimeragenesis from Cannabis sativa
MAATTNQTEPPESDNHSVATKILNFGKACWKLQRPYTIIAFTSCACGLFGKELLHNTNLISWSLMFKAFFF
LVAILCIASFTTTINQIYDLHIDRINKPDLPLASGEISVNTAWIMSIIVALFGLIITIKMKGGPLYIFGYCFGIFGGI
VYSVPPFRWKQNPSTAFLLNFLAHIITNFTFYYASRAALGLPFELRPSFTFLLAFMKSMGSALALIKDASDV
EGDTKFGISTLASKYGSRNLTLFCSGIVLLSYVAAILAGIIWPQAFNSNVMLLSHAILAFWLILQTRDFALTNY
DPEAGRRFYEFMWKLYYAEYLVYVFI
SEQ ID NO: 60 - Parent for Chimeragenesis from Cannabis sativa
MTDTANQTEPPESNTKYSVVTKILSFGHTCWKLQRPYTFIGVISCACGLFGRELFHNTNLLSWSLMLKAFS
SLMVILSVNLCTNIINQITDLDIDRINKPDLPLASGEMSIETAWIMSIIVALTGLILTIKLNCGPLFISLYCVSILV
GALYSVPPFRWKQNPNTAFSSYFMGLVIVNFTCYYASRAAFGLPFEMSPPFTFILAFVKSMGSALFLCKD
VSDIEGDSKHGISTLATRYGAKNITFLCSGIVLLTYVSAILAAIIWPQAFKSNVMLLSHATLAFWLIFQTREFA
LTNYNPEAGRKFYEFMWKLHYAEYLVYVFI
SEQ ID NO: 61 - Parent for Chimeragenesis from Humulus lupulus
MDQRGNSIRASAQIEDRPPESGNLSALTNVKDFVSVCWEYVRPYTAKGVIICSSCLFGRELLENPNLFSW
PLIFRALLGMLAILGSCFYTAGINQIFDMDIDRINKPDLPLVSGRISVESAWLLTLSPAIIGFILILKLNSGPLLT
SLYCLAILSGTIYSVPPFRWKKNPITAFLCILMIHAGLNFSVYYASRAALGLAFVWSPSFSFITAFITFMTLTL
ASSKDLSDINGDRKFGVETFATKLGAKNITLLGTGLLLLNYVAAISTAIIWPKAFKSNIMLLSHAILAFSLFFQ
ARELDRTNYTPEACKSFYEFIWILFSAEYVVYLFI
SEQ ID NO: 62 - Parent for Chimeragenesis from Humulus lupulus
MPNSLTAWSHQSEFPSTIVTKGSNFGHASWKFVRPIPFVAVSIICTSLFGAELLKNPNLFSWQLMFDAFQ
GLVVILLYHIYINGLNQIYDLESDRINKPDLPLAAEEMSVKSAWFLTIFSAVASLLLMIKLKCGLFLTCMYCCY
LVIGAMYSVPPFRWKMNTFTSTLWNFSEIGIGINFLINYASRATLGLPFQWRPPFTFIIGFVSTLSIILSILKDV
PDVEGDKKVGMSTLPVIFGARTIVLVGSGFFLLNYVAAIGVAIMWPQAFKGYIMIPAHAIFASALIFKTWLLD
KANYAKEASDSYYHFLWFLMIAEYILYPFIST
SEQ ID NO: 63 - CBGaS (synthetic)
MAGSDQIEGSPHHESDNSIATKILNFGHTCWKLQRPYVVKGMISIACGLFGRELFNNRHLFSWGLMWKA
FFALVPILSFNFFAAIMNQIYDVDIDRINKPDLPLVSGEMSIETAWILSIIVALTGLIVTIKLKSAPLFVFIYIFGIF
AGFAYSVPPIRWKQYPFTNFLITISSHVGLAFTSYSATTSALGLPFVWRPAFSFIIAFMTVMGMTIAFCKDV
SDIEGDSKHGISTLATRYGAKNITFLCSGIVLLTYVSAILAAIIWPQVFKSNIMILSHAILAFCLIFQTRELALAN
YASAPSRQFFEFIWLLYYAEYFVYVFI
SEQ ID NO: 64 - CBGaS (synthetic)
MAGSDQIEGSPHHESDNSIATKILNFGHTCWKLQRPYVVKGMISIACGLFGRELFNNRHLFSWGLMWKA
FFALVPILSFNFFAAIMNQIYDVDIDRINKPDLPLVSGEMSIETAWILSIIVALTGLIVTIKLKSAPLFVFIYIFGIF
AGFAYSVPPIRWKQYPFTNFLITISSHVGLAFTSYSATTSALGLPFVWRPAFSFIIAFMTVMGMTIAFLKDVP
DVEGDKKVGMSTLPVIFGARTIVLVGSGFFLLNYVAAIGVAIMWPQAFKGYIMIPAHAIFASALIFKTRELAL
ANYASAPSRQFFEFIWLLYYAEYFVYVFI
SEQ ID NO: 65 - PDC from Zymomonas mobilis
MSYTVGTYLAERLVQIGLKHHFAVAGDYNLVLLDNLLLNKNMEQVYCCNELNCGFSAEGYARAKGAAAA
VVTYSVGALSAFDAIGGAYAENLPVILISGAPNNNDHAAGHVLHHALGKTDYHYQLEMAKNITAAAEAIYTP
EEAPAKIDHVIKTALREKKPVYLEIACNIASMPCAAPGPASALFNDEASDEASLNAAVEETLKFIANRDKVA
VLVGSKLRAAGAEEAAVKFADALGGAVATMAAAKSFFPEENPHYIGTSWGEVSYPGVEKTMKEADAVIAL
APVFNDYSTTGWTDIPDPKKLVLAEPRSVVVNGIRFPSVHLKDYLTRLAQKVSKKTGALDFFKSLNAGELK
KAAPADPSAPLVNAEIARQVEALLTPNTTVIAETGDSWFNAQRMKLPNGARVEYEMQWGHIGWSVPAAF
GYAVGAPERRNILMVGDGSFQLTAQEVAQMVRLKLPVIIFLINNYGYTIEVMIHDGPYNNIKNWDYAGLME
VFNGNGGYDSGAGKGLKAKTGGELAEAIKVALANTDGPTLIECFIGREDCTEELVKWGKRVAAANSRKPV
NKLL
SEQ ID NO: 66 - ACS1 from Saccharomyces cerevisiae
MSPSAVQSSKLEEQSSEIDKLKAKMSQSASTAQQKKEHEYEHLTSVKIVPQRPISDRLQPAIATHYSPHLD
GLQDYQRLHKESIEDPAKFFGSKATQFLNWSKPFDKVFIPDSKTGRPSFQNNAWFLNGQLNACYNCVDR
HALKTPNKKAIIFEGDEPGQGYSITYKELLEEVCQVAQVLTYSMGVRKGDTVAVYMPMVPEAIITLLAISRI
GAIHSVVFAGFSSNSLRDRINDGDSKVVITTDESNRGGKVIETKRIVDDALRETPGVRHVLVYRKTNNPSV
AFHAPRDLDWATEKKKYKTYYPCTPVDSEDPLFLLYTSGSTGAPKGVQHSTAGYLLGALLTMRYTFDTH
QEDVFFTAGDIGWITGHTYVVYGPLLYGCATLVFEGTPAYPNYSRYWDIIDEHKVTQFYVAPTALRLLKRA
GDSYIENHSLKSLRCLGSVGEPIAAEVWEWYSEKIGKNEIPIVDTYWQTESGSHLVTPLAGGVTPMKPGS
ASFPFFGIDAVVLDPNTGEELNTSHAEGVLAVKAAWPSFARTIWKNHDRYLDTYLNPYPGYYFTGDGAAK
DKDGYIWILGRVDDVVNVSGHRLSTAEIEAAIIEDPIVAECAVVGFNDDLTGQAVAAFVVLKNKSNWSTAT
DDELQDIKKHLVFTVRKDIGPFAAPKLIILVDDLPKTRSGKIMRRILRKILAGESDQLGDVSTLSNPGIVRHLI
DSVKL
SEQ ID NO: 67 - ALD6 from Saccharomyces cerevisiae
MTKLHFDTAEPVKITLPNGLTYEQPTGLFINNKFMKAQDGKTYPVEDPSTENTVCEVSSATTEDVEYAIEC
ADRAFHDTEWATQDPRERGRLLSKLADELESQIDLVSSIEALDNGKTLALARGDVTIAINCLRDAAAYADK
VNGRTINTGDGYMNFTTLEPIGVCGQIIPWNFPIMMLAWKIAPALAMGNVCILKPAAVTPLNALYFASLCKK
VGIPAGVVNIVPGPGRTVGAALTNDPRIRKLAFTGSTEVGKSVAVDSSESNLKKITLELGGKSAHLVFDDA
NIKKTLPNLVNGIFKNAGQICSSGSRIYVQEGIYDELLAAFKAYLETEIKVGNPFDKANFQGAITNRQQFDTI
MNYIDIGKKEGAKILTGGEKVGDKGYFIRPTVFYDVNEDMRIVKEEIFGPVVTVAKFKTLEEGVEMANSSE
FGLGSGIETESLSTGLKVAKMLKAGTVWINTYNDFDSRVPFGGVKQSGYGREMGEEVYHAYTEVKAVRI
KL
SEQ ID NO: 68 - ERG10 from Saccharomyces cerevisiae
MSQNVYIVSTARTPIGSFQGSLSSKTAVELGAVALKGALAKVPELDASKDFDEIIFGNVLSANLGQAPARQ
VALAAGLSNHIVASTVNKVCASAMKAIILGAQSIKCGNADVVVAGGCESMTNAPYYMPAARAGAKFGQTV
LVDGVERDGLNDAYDGLAMGVHAEKCARDWDITREQQDNFAIESYQKSQKSQKEGKFDNEIVPVTIKGF
RGKPDTQVTKDEEPARLHVEKLRSARTVFQKENGTVTAANASPINDGAAAVILVSEKVLKEKNLKPLAIIKG
WGEAAHQPADFTWAPSLAVPKALKHAGIEDINSVDYFEFNEAFSVVGLVNTKILKLDPSKVNVYGGAVAL
GHPLGCSGARVVVTLLSILQQEGGKIGVAAICNGGGGASSIVIEKI
SEQ ID NO: 69 - ERG13 from Saccharomyces cerevisiae
MKLSTKLCWCGIKGRLRPQKQQQLHNTNLQMTELKKQKTAEQKTRPQNVGIKGIQIYIPTQCVNQSELEK
FDGVSQGKYTIGLGQTNMSFVNDREDIYSMSLTVLSKLIKSYNIDTNKIGRLEVGTETLIDKSKSVKSVLMQ
LFGENTDVEGIDTLNACYGGTNALFNSLNWIESNAWDGRDAIVVCGDIAIYDKGAARPTGGAGTVAMWIG
PDAPIVFDSVRASYMEHAYDFYKPDFTSEYPYVDGHFSLTCYVKALDQVYKSYSKKAISKGLVSDPAGSD
ALNVLKYFDYNVFHVPTCKLVTKSYGRLLYNDFRANPQLFPEVDAELATRDYDESLTDKNIEKTFVNVAKP
FHKERVAQSLIVPTNTGNMYTASVYAAFASLLNYVGSDDLQGKRVGLFSYGSGLAASLYSCKIVGDVQHII
KELDITNKLAKRITETPKDYEAAIELRENAHLKKNFKPQGSIEHLQSGVYYLTNIDDKFRRSYDVKK
SEQ ID NO: 70 - HMGR-t from Saccharomyces cerevisiae
MAADQLVKTEVTKKSFTAPVQKASTPVLTNKTVISGSKVKSLSSAQSSSSGPSSSSEEDDSRDIESLDKKI
RPLEELEALLSSGNTKQLKNKEVAALVIHGKLPLYALEKKLGDTTRAVAVRRKALSILAEAPVLASDRLPYK
NYDYDRVFGACCENVIGYMPLPVGVIGPLVIDGTSYHIPMATTEGCLVASAMRGCKAINAGGGATTVLTK
DGMTRGPVVRFPTLKRSGACKIWLDSEEGQNAIKKAFNSTSRFARLQHIQTCLAGDLLFMRFRTTTGDA
MGMNMISKGVEYSLKQMVEEYGWEDMEVVSVSGNYCTDKKPAAINWIEGRGKSVVAEATIPGDVVRKV
LKSDVSALVELNIAKNLVGSAMAGSVGGFNAHAANLVTAVFLALGQDPAQNVESSNCITLMKEVDGDLRI
SVSMPSIEVGTIGGGTVLEPQGAMLDLLGVRGPHATAPGTNARQLARIVACAVLAGELSLCAALAAGHLV
QSHMTHNRKPAEPTKPNNLDATDINRLKDGSVTCIKS
SEQ ID NO: 71 - ERG12 from Saccharomyces cerevisiae
MSLPFLTSAPGKVIIFGEHSAVYNKPAVAASVSALRTYLLISESSAPDTIELDFPDISFNHKWSINDFNAITED
QVNSQKLAKAQQATDGLSQELVSLLDPLLAQLSESFHYHAAFCFLYMFVCLCPHAKNIKFSLKSTLPIGAG
LGSSASISVSLALAMAYLGGLIGSNDLEKLSENDKHIVNQWAFIGEKCIHGTPSGIDNAVATYGNALLFEKD
SHNGTINTNNFKFLDDFPAIPMILTYTRIPRSTKDLVARVRVLVTEKFPEVMKPILDAMGECALQGLEIMTKL
SKCKGTDDEAVETNNELYEQLLELIRINHGLLVSIGVSHPGLELIKNLSDDLRIGSTKLTGAGGGGCSLTLL
RRDITQEQIDSFKKKLQDDFSYETFETDLGGTGCCLLSAKNLNKDLKIKSLVFQLFENKTTTKQQIDDLLLP
GNTNLPWTS
SEQ ID NO: 72 - ERG8 from Saccharomyces cerevisiae
MSELRAFSAPGKALLAGGYLVLDPKYEAFVVGLSARMHAVAHPYGSLQESDKFEVRVKSKQFKDGEWL
YHISPKTGFIPVSIGGSKNPFIEKVIANVFSYFKPNMDDYCNRNLFVIDIFSDDAYHSQEDSVTEHRGNRRL
SFHSHRIEEVPKTGLGSSAGLVTVLTTALASFFVSDLENNVDKYREVIHNLSQVAHCQAQGKIGSGFDVAA
AAYGSIRYRRFPPALISNLPDIGSATYGSKLAHLVNEEDWNITIKSNHLPSGLTLWMGDIKNGSETVKLVQK
VKNWYDSHMPESLKIYTELDHANSRFMDGLSKLDRLHETHDDYSDQIFESLERNDCTCQKYPEITEVRDA
VATIRRSFRKITKESGADIEPPVQTSLLDDCQTLKGVLTCLIPGAGGYDAIAVIAKQDVDLRAQTADDKRFS
KVQWLDVTQADWGVRKEKDPETYLDK
SEQ ID NO: 73 - MVD1 from Saccharomyces cerevisiae
MTVYTASVTAPVNIATLKYWGKRDTKLNLPTNSSISVTLSQDDLRTLTSAATAPEFERDTLWLNGEPHSID
NERTQNCLRDLRQLRKEMESKDASLPTLSQWKLHIVSENNFPTAAGLASSAAGFAALVSAIAKLYQLPQS
TSEISRIARKGSGSACRSLFGGYVAWEMGKAEDGHDSMAVQIADSSDWPQMKACVLVVSDIKKDVSSTQ
GMQLTVATSELFKERIEHVVPKRFEVMRKAIVEKDFATFAKETMMDSNSFHATCLDSFPPIFYMNDTSKRII
SWCHTINQFYGETIVAYTFDAGPNAVLYYLAENESKLFAFIYKLFGSVPGWDKKFTTEQLEAFNHQFESSN
FTARELDLELQKDVARVILTQVGSGPQETNESLIDAKTGLPKE
SEQ ID NO: 74 - IDI1 from Saccharomyces cerevisiae
MTADNNSMPHGAVSSYAKLVQNQTPEDILEEFPEIIPLQQRPNTRSSETSNDESGETCFSGHDEEQIKLM
NENCIVLDWDDNAIGAGTKKVCHLMENIEKGLLHRAFSVFIFNEQGELLLQQRATEKITFPDLWTNTCCSH
PLCIDDELGLKGKLDDKIKGAITAAVRKLDHELGIPEDETKTRGKFHFLNRIHYMAPSNEPWGEHEIDYILFY
KINAKENLTVNPNVNEVRDFKWVSPNDLKTMFADPSYKFTPWFKIICENYLFNWWEQLDDLSEVENDRQI
HRML
SEQ ID NO: 75 - GPPS from Streptomyces aculeolatus
MTTEVTSFTGAGPHPAASVRRITDDLLQRVEDKLASFLTAERDRYAAMDERALAAVDALTDLVTSGGKRV
RPTFCITGYLAAGGDAGDPGIVAAAAGLEMLHVSALIHDDILDNSAQRRGKPTIHTLYGDLHDSHGWRGE
SRRFGEGIGILIGNLALVYSQELVCQAPPAVLAEWHRLCSEVNIGQCLDVCAAAEFSADPELSRLVALIKS
GRYTIHRPLVMGANAASRPDLAAAYVEYGEAVGEAFQLRDDLLDAFGDSTETGKPTGLDFTQHKMTLLL
GWAMQRDTHIRTLMTEPGHTPEEVRRRLEDTEVPKDVERHIADLVEQGRAAIADAPIDPQWRQELADMA
VRAAYRTN

1-270. (canceled)

271. A non-naturally occurring CBGaS enzyme:
(a) having an amino acid sequence that is at least 90% identical to the amino acid sequence of SEQ ID NO: 55, SEQ ID NO:56, SEQ ID NO:57, or SEQ ID NO:58;
(b) having an amino acid sequence that is at least 90% identical to the amino acid sequence of SEQ ID NO: 63, optionally wherein the CBGaS has one or more amino acid substitutions relative to the amino acid sequence of SEQ ID NO: 63 selected from I109T, F119L, S245L, S247Y, M270T, C280L, S295D, V314L, A324F, and S361I; or
(c) having an amino acid sequence that is at least 90% identical to the amino acid sequence of SEQ ID NO; 64, optionally wherein the CBGaS has one or more amino acid substitutions relative to the amino acid sequence of SEQ ID NO: 64 selected from M275S, M275T, T276C, T276F, K291H, V292Y, V292H, V292F, G310C, F314N, A331C, A331T, and A347I.

272. A nucleic acid encoding the enzyme of claim 271.

273. A host cell comprising the nucleic acid of claim 272.

274. A host cell comprising one or more heterologous nucleic acids that each, independently, encode
(a) an acyl activating enzyme (AAE) having an amino acid sequence that is at least 90% identical to the amino acid sequence of any one of SEQ ID NOS: 1-5 and 7-24, and/or
(b) a tetraketide synthase (TKS) having an amino acid sequence that is at least 90% identical to the amino acid sequence of any one of SEQ ID NOS: 25 and 27-43, and/or
(c) a cannabigerolic acid synthase (CBGaS) having an amino acid sequence that is at least 90% identical to the amino acid sequence of any one of SEQ ID NOS: 55-58, 63, and 64, and/or
(d) an olivetolic acid cyclase (OAC) having an amino acid sequence that is at least 90% identical to the amino acid sequence of any one of SEQ ID NOS: 45-52.

275. The host cell of claim 274, wherein the host cell comprises a heterologous nucleic acid that encodes an AAE having an amino acid sequence that is at least 90% identical to the amino acid sequence of any one of SEQ ID NOS: 1-4.

276. The host cell of claim 274, wherein the host cell comprises a heterologous nucleic acid that encodes a TKS having an amino acid sequence that is at least 90% identical to the amino acid sequence of any one of SEQ ID NOS: 25 and 34-39.

277. The host cell of claim 274, wherein the CBGaS has one or more amino acid substitutions relative to the amino acid sequence of:
(a) SEQ ID NO: 55, wherein the one or more amino acid substitutions are selected from M88I, V133I, S141Y, Y319L, and L324F;
(b) SEQ ID NO: 56, wherein the one or more amino acid substitutions are selected from P7K, P7T, T11T, H49C, M83V, A89A, N93V, A131G, V149F, A176V, R196F, T202A, V242L, T248A, C249F, A257Y, A257F, V262L, N264Y, N264F, L276T, L276P, A279C, A279S, A282P, N309F, M311L, S312L, Y319L, I324E, I324K, L325P, and L325A;
(c) SEQ ID NO: 63, wherein the one or more amino acid substitutions are selected from I109T, F119L, S245L, S247Y, M270T, C280L, S295D, V314L, A324F, and S361I; or
(d) SEQ ID NO: 64, wherein the one or more amino acid substitutions are selected from M275S, M275T, T276C, T276F, K291H, V292Y, V292H, V292F, G310C, F314N, A331C, A331T, and A347I.

278. The host cell of claim 274, wherein the OAC has one or more amino acid substitutions relative to the amino acid sequence of SEQ ID NO: 44, wherein the one or more amino acid substitutions are selected from A2S, L9I, K12S, E14S, F23L, V28L, T47R, Q48R, K49R, S87H, F88Y, and L92Y.

279. The host cell of claim 274, wherein the host cell further comprises one or more heterologous nucleic acids that each, independently, encode an enzyme of the mevalonate biosynthetic pathway, optionally wherein the enzyme of the mevalonate biosynthetic pathway is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase, optionally wherein:
(a) the acetyl-CoA thiolase has an amino acid sequence that is at least 90% identical to the amino acid sequence of SEQ ID NO: 68;
(b) the HMG-COA synthase has an amino acid sequence that is at least 90% identical to the amino acid sequence of SEQ ID NO: 69;
(c) the HMG-CoA reductase has an amino acid sequence that is at least 90% identical to the amino acid sequence of SEQ ID NO: 70;
(d) the mevalonate kinase has an amino acid sequence that is at least 90% identical to the amino acid sequence of SEQ ID NO: 71;
(e) the phosphomevalonate kinase has an amino acid sequence that is at least 90% identical to the amino acid sequence of SEQ ID NO: 72; or
(f) the mevalonate pyrophosphate decarboxylase has an amino acid sequence that is at least 90% identical to the amino acid sequence of SEQ ID NO: 73; or
(g) the IPP:DMAPP isomerase has an amino acid sequence that is at least 90% identical to the amino acid sequence of SEQ ID NO: 74.

280. The host cell of claim 274, wherein the host cell further comprises a heterologous nucleic acid that encodes a geranyl pyrophosphate (GPP) synthase, optionally wherein the GPP synthase has an amino acid sequence that is at least 90% identical to the amino acid sequence of SEQ ID NO: 75.

281. The host cell of claim 274, wherein the host cell further comprises one or more heterologous nucleic acids that each, independently, encode an acetyl-CoA synthase, an aldehyde dehydrogenase, and/or a pyruvate decarboxylase, optionally wherein:
(a) the acetyl-CoA synthase has an amino acid sequence that is at least 90% identical to the amino acid sequence of SEQ ID NO: 66;
(b) the aldehyde dehydrogenase has an amino acid sequence that is at least 90% identical to the amino acid sequence of SEQ ID NO: 67; or
(c) the pyruvate decarboxylase has an amino acid sequence that is at least 90% identical to the amino acid sequence of SEQ ID NO: 65.

282. The host cell of claim 274, wherein expression of one or more of the heterologous nucleic acids is regulated by an exogenous agent.

283. A mixture comprising the host cell of claim 274 and a culture medium.

284. A fermentation composition comprising a mixture of claim 283.

285. A method of genetically modifying a host cell, the method comprising introducing into the host cell one or more heterologous nucleic acids that each, independently, encode
(a) an AAE having an amino acid sequence that is at least 90% identical to the amino acid sequence of any one of SEQ ID NOS: 1-5 and 7-24, and/or
(b) a TKS having an amino acid sequence that is at least 90% identical to the amino acid sequence of any one of SEQ ID NOS: 25 and 27-43, and/or
(c) a CBGaS having an amino acid sequence that is at least 90% identical to the amino acid sequence of any one of SEQ ID NOS: 55-58, 63, and 64, and/or
(d) an OAC having an amino acid sequence that is at least 90% identical to the amino acid sequence of any one of SEQ ID NO: 45-52.


SEQUENCE LISTING (75 sequences)

SEQ ID NO: 1 - protein, 541 aa, Pseudonocardia sp.
  source 1..541: note = N23
MTAAQAPDPAGVPLVERTVPRMLARSAALDPDRPFVVTRERTWSHTDAHRIVATLAAAFTDRGIGQGSRVAVMMPTSPRHVWLLLALAHLRAVPVALNPDASGEVLRYFVADSECVLGVVDQERAAAFATAAGPDGPPAIVLPPGADDLGELGSAGPGPLDPGAASFSDTFVVLYTSGSTGMPKATAVTHAQVITCGAVFTDRLGLGPADRLYTCLPLFHINATAYSLSGALVSGASLALGPHFSATTFWDDVADLGATEVNAMGSMVRILQSRPPRPAERAHRVRTMFVAPLPPDAVELSERFGLDFATCYAQTEWLPSSMTRPGEGYGRPGATGPVLPWTEVRIVGDDDRPLPAGQTGEIILRPRDPYTTFQGYLGKPQETVDAWRNLWFHTGDLGDIGPDGWLHYRGRRKDVIRRRGENIPATVVEDLLAGHPDIAEVAAVSVPAHISEEEIFAFVVPGAGAALTTADVEAHAHAVLPRYMVPSYLALVPDLPRTATNKIAKVELTERARAAVEGTGDPADAPTRTSAADRVVVPAAE

SEQ ID NO: 2 - protein, 548 aa, Pseudomonas putida
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 3 - protein, 549 aa, Streptomyces sp.
  source 1..549: note = ADI96-02
MLSTMQDVPLTVTRILQHGMTIHGKSQVTTWTGEPEPHRRTFAEIGARATRLAHALRDELGIDGDQRVATLMWNNAEHVEAYLAVPSMGAVLHTLNLRLPAEQLIWIVNHADDKVVIVNGSLLPLLVPLLPHLPTVEHVVVSGPGDRSALAGVAPRVHEYEELIADRPTTYDWPELDERQAAAMCYTSGTTGDPKGVVYSHRSVYLHSMQVNMTESMGLTDKDTTLVVVPQFHVNAWGLPHATFMAGVNMLMPDRFLQPAPLADMIERERPTHAAAVPTIWQGLLAEVTAHPRDLTSMASVTIGGAACPPSLMEAYDKLGVRLCHAWGMTETSPLGTMANPPAGLSAEEEWPYRVTQGRFPAGVEARLVGPAGDHLPWDGRSAGELEVRGAWIAGAYYGGADGEHLRPEDKFSADGWLKTGDVGVISADGFLTLTDRAKDVIKSGGEWISSVELENALMAHPDVAEAAVVAVPDEKWGERPLATVVLKEGAEVGYEALKVFLADSGIAKWQLPERWTVIPAVPKTSVGKFDKKVIRKQYADGELDITQL

SEQ ID NO: 4 - protein, 560 aa, Erythrobacter citreus
  source 1..560: note = LAMA 915
MSRAECRDRLTAPGERFEIETIDIRGVPTRVWKHAPTNMRQVAMAARTHGDRLFAIYEDERVTYEAWFRAVARMAAELRERGVAKGDRVALAMRNLPEWPVAFFAATTIGAICVPLNAWWTGPELAFGLANSGAKLLVCDAERWERIAPHRGELPDLEHALVSRSDAPLEGAEQLEDLLGTPKDYAALPSAALPQVDIDPEDEATIFYTSGTTGQPKGALGTHRNLCTNIMSSAYNGAIAFLRRGEEPPAPVQKVGLTVIPLFHVTACSAGLMGYVVAGHTMVFMHKWDPVKAFQLIEREKVNLTGGVPTIAWQLLEHPERANYDLSSLEAVAYGGAPAAPELVRKIHEEFGALPANGWGMTETMATVTGHSSEDYLNRPDSCGPPVAVADLKIVGDDGVTELPVGEVGELWARGPMVVKGYWNRPEATAETFVDGWVRTGDLARLDEEGWCYIVDRAKDMIIRGGENIYSSEVENVLYDHPAVTDAALVAIAHPTLGEEPAAVVHLAPGMSATEDELREWVAARLAKFKVPVRIAFVQDTLPRNANGKILKKDLGAFFA

SEQ ID NO: 5 - protein, 700 aa, Saccharomyces cerevisiae
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 6 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 7 - protein, 665 aa, Citreicella sp.
  source 1..665: note = SE45
MSLAADNVLLVEEGRPATAEHPSAGPVYRCKYAKDGLLDLPTDIDSPWQFFSEAVKKYPNEQMLGQRVTTDSKVGPYTWITYKEAHDAAIRIGSAIRSRGVDPGHCCGIYGANCPEWIIAMEACMSQGITYVPLYDSLGVNAVEFIINHAEVSLVFVQEKTVSSILSCQKGCSSNLKTIVSFGEVSSTQKEEAKNQCVSLFSWNEFSLMGNLDEANLPRKRKTDICTIMYTSGTTGEPKGVILNNAAISVQVLSIDKMLEVTDRSCDTSDVFFSYLPLAHCYDQVMEIYFLSRGSSVGYWRGDIRYLMDDVQALKPTVFCGVPRVYDKLYAGIMQKISASGLIRKKLFDFAYNYKLGNMRKGFSQEEASPRLDRLMFDKIKEALGGRAHMLLSGAAPLPRHVEEFLRIIPASNLSQGYGLTESCGGSFTTLAGVFSMVGTVGVPMPTVEARLVSVPEMGYDAFSADVPRGEICLRGNSMFSGYHKRQDLTDQVLIDGWFHTGDIGEWQEDGSMKIIDRKKNIFKLSQGEYVAVENLENTYSRCPLIAQIWVYGNSFESFLVGVVVPDRKAIEDWAKLNYQSPNDFESLCQNLKAQKYFLDELNSTAKQYQLKGFEMLKAIHLEPNPFDIERDLITPTFKLKRPQLLQHYKGIVDQLYSEAKRSMA

SEQ ID NO: 8 - protein, 572 aa, Bacillus subtilis
  source 1..572: note = (strain 168)
MDNLVLCEANNVPLTPITFLKRASECYPNRTSIIYGQTRFTWPQTYDRCCRLAASLLSLNITRNDVVSILAPNVPAMYEMHFSVPMTGAVLNPINTRLDAKTIAIILRHAEPKILFVDYEFAPLIQEVLRLIPTYQSQPHPRIILINEIDSTTKPFSKELDYEGLIRKGEPTPSSSASMFRVHNEHDPISLNYTSGTTADPKGVVISHQGAYLSALSSIIGWEMGIFPVYLWTLPMFHCNGWTHTWSVAARGGTNVCIRHVTAPEIYKNIELHGVTHMSCVPTVFRFLLEGSRTDQSPKSSPVQVLTGGSSPPAVLIKKVEQLGFHVMHGYGLTEATGPVLFCEWQDEWNKLPEHQQIELQQRQGVRNLTLADVDVKNTKTLESVPRDGKTMGEIVIKGSSLMKGYLKNPKATSEAFKHGWLNTGDIGVIHPDGYVEIKDRSKDIIISGGENISSIEVEKVLYMYQEVLEAAVVAMPHPLWGETPCAFVVLKKGEEGLVTSEGDLIKYCRENMPHFMCPKKVVFFQELPKNSNGKILKSKLRDIAKALVVREDDAGSKKVHQRSIEHVSSRL

SEQ ID NO: 9 - protein, 504 aa, Bhargavaea cecembensis
  source 1..504: note = DSE10
MYTDHGWIMKRADITPDGTALIDVHTGQRWTYRELAGRTAAYMEQFRSAGLRKGERVAVLSHNRIDLFAVLFACAGRGLIYVPMNWRLSESELRYIVSDSGPSLLLHDHEHAGRAAGLGIPAALLDSVPATSVNLRTEQAAGRLDDPWMMIYTGGTTGRPKGVVLTFESVNWNAINTIISWNLSARDCTLNYMPLFHTGGLNALSLPILMAGGTVVIGRKFDPEEAIRALNDYRTTISLFVPTMHQAMLDTDLFWESDFPTVDVFLSGGAPCPQTVYDAYRKKGVRFREGYGMTEAGPNNFIIDPDTAMRKRGAVGKSMQFNEVRILDAKGRPCRAGEVGELHLRGRHLFSHYWNNEEATQEALKEGWFSTGDLASRDEDGDYFIVGRKKEMIISGGENIYPQEVEQCLIGHDGVREIAVIGIADRKWGERVVAFIVAQPGNIPKTEELLKHCAQTLGSYKVPKDFFFVQELPITDIGKIDKKQLAIMAEELKKEEMQHPGQSG

SEQ ID NO: 10 - protein, 694 aa, Saccharomyces cerevisiae
MTEQYSVAVGEAANEHETAPRRNIRVKDQPLIRPINSSASTLYEFALECFTKGGKRDGMAWRDIIDIHETKKTIVKRVDGKDKPIEKTWLYYELTPYITMTYEEMICVMHDIGRGLIKIGVKPNGENKFHIFASTSHKWMKTFLGCMSQGIPVVTAYDTLGESGLIHSMVETDSVAIFTDNQLLSKLAVPLKTAKNVKFVIHNEPIDPSDKRQNGKLYKAAKDAVDKIKEVRPDIKIYSFDEIIEIGKKAKDEVELHFPKPEDPACIMYTSGSTGTPKGVVLTHYNIVAGIGGVGHNVIGWIGPTDRIIAFLPLAHIFELTFEFEAFYWNGILGYANVKTLTPTSTRNCQGDLMEFKPTVMVGVAAVWETVRKGILAKINELPGWSQTLFWTVYALKERNIPCSGLLSGLIFKRIREATGGNLRFILNGGSAISIDAQKFLSNLLCPMLIGYGLTEGVANACVLEPEHFDYGIAGDLVGTITAKLVDVEDLGYFAKNNQGELLFKGAPICSEYYKNPEETAAAFTDDGWFRTGDIAEWTPKGQVKIIDRKKNLVKTLNGEYIALEKLESIYRSNPYVQNICVYADENKVKPVGIVVPNLGHLSKLAIELGIMVPGEDVESYIHEKKLQDAVCKDMLSTAKSQGLNGIELLCGIVFFEEEWTPENGLVTSAQKLKRRDILAAVKPDVERVYKENT

SEQ ID NO: 11 - protein, 602 aa, unidentified
  source 1..602: note = Deltaproteobacteria bacterium ADurb.Bin022
MHKFTLDKPDNLVDWWGESVTRFADRPLFGTKNKEGVYKWATYKEIGNRIDNLRAGLTQLGIGKDDVVGIIANNRPEWAVIGFATWGCLARYVPMYEAELVQVWKYIINDSGAKVLFVSNPAIYEKIKDFPKDIPTLKHIFIIESDGDNSMASLEKKGAAKPVAPKSPKAEDVAELIYTSGTTGNPKGVLLMHMNFTSNSHAGLKMYPELYENEVVSLTILPWAHVFGQTAELFAIIRLGGRMGLIESTKTIINDIVQIKPTFIIAVPTVFNRIYDGLWNKMNKDGGLARALFVMGVEAAKKKRILAEKGQSDLMTNFKVAVADKIVFKKIRERMGGRMLGSMTGSAAMNVEISKFFFDIGIPIYDCYGLTETSPGITMNGSQAYRIGSVGRPIDKVKVVIDSSVVEEGATDGEIIAYGPNVMKGYHNRPEDTKAALTPDGGFRTGDRGRLDKDGYLFITGRIKEQYKLENGKFCFPVSLEENICLASFVQQAVVYGLNRPYNVCIVVPDFDVLLDYAKEKGLPTDIKTLVEREDIIHMISEAVTGQLKGKFGGYEIPKKFIILPEAFSLDNGMLTQTMKLKRKVILDKLNDRIEALYKEDK

SEQ ID NO: 12 - protein, 534 aa, Alcaligenes xylosoxydans
  source 1..534: note = (Achromobacter xylosoxidans)
MYSRIHEPHACTLTDALREWAASRPAAPWLEDSQGIAFTVGQAFTSSQRFASFLHHQLGVQPEERVGVFMSNSCAMVATTFGIGYLRATAVMLNTELRSSFLRHQLNDCQLATIVVDSALVEHVASLADELPHLRTLVVVGDAPAAVPERWRQVAWMDSSACAPWEGPAPRPEDIFCIMYTSGTTGPSKGVLMPHCHCALLGLGAIRSLEITEADKYYICLPLFHANGLFMQLGATVLAGIPAFLKQRFSASTWLADIRRSGATLTNHLGTTAMFVINQPPTEQDRDHRLRASLSAPNPAQHEAVFRERFGVKDVLSGFGMTEVGIPIWGRIGHAAPNAAGWAHEDRFEICIADPETDVPVLAGQVGEILVRPKVPFGFMAGYLNVPAKTVEAWRNLWFHTGDAGTRDEQGLITFVDRIKDCIRRRGENISATEVEVVVGQLPGVHEVAAYAVPAQGAGGEDEVMLALVPSEGAALDMADIVRQASAQLPRFAKPRYLRQMDSLPKTATGKIQRAVLRQQGSAGAYDAEAAPAR

SEQ ID NO: 13 - protein, 518 aa, Novosphingobium sp.
  source 1..518: note = MD-1
MQFTQGLERAVQHHPDVTATICRARSQTFAELYERVTGLAGCLASRSLAKGARIAVLALNSDHYLEVYLATAWAGGVIVPVNFRWSPAEIAYSLNDAGCVALMVDQHHAALVPTLREQCPGLQHIFLMGGTEESDDLPGLDALIAAAEPLQNAGAGGDDLLGIFYTGGTTGRPKGVMLSHANLCSSGLSMLAEGVFNEGAVGLHVAPMFHLADMLLTTCLVLRGCTHVMLPAFSPDAVLDHVARFGVTDTLVVPAMLQAIVDHPAIGNFDTSSLCNILYGASPASETLLRRTMAAFPDVRLTQGYGMTESAAFICALPWHQHVVDNDGPNRLRAAGRSTFDVHLQIVDPDDRELPRGEIGEIIVKGPNVMQGYYNMPEATAETLRGGWLHTGDMAWMDEEGYVFIVDRAKDMIISGGENIYSAEVENAVASHPAVAANAVIGIPHEQMGEAVHVALVLRPGSELSLEALQAHCRALIAGYKVPRSMEVRPSLPLSGAGKILKTELREPFWKGRDRAVG

SEQ ID NO: 14 - protein, 541 aa, Thermus thermophilus
  source 1..541: note = (strain HB8 / ATCC 27634 / DSM 579)
MEGERMNAFPSTMMDEELNLWDFLERAAALFGRKEVVSRLHTGEVHRTTYAEVYQRARRLMGGLRALGVGVGDRVATLGFNHFRHLEAYFAVPGMGAVLHTANPRLSPKEIAYILNHAEDKVLLFDPNLLPLVEAIRGELKTVQHFVVMDEKAPEGYLAYEEALGEEADPVRVPERAACGMAYTTGTTGLPKGVVYSHRALVLHSLAASLVDGTALSEKDVVLPVVPMFHVNAWCLPYAATLVGAKQVLPGPRLDPASLVELFDGEGVTFTAGVPTVWLALADYLESTGHRLKTLRRLVVGGSAAPRSLIARFERMGVEVRQGYGLTETSPVVVQNFVKSHLESLSEEEKLTLKAKTGLPIPLVRLRVADEEGRPVPKDGKALGEVQLKGPWITGGYYGNEEATRSALTPDGFFRTGDIAVWDEEGYVEIKDRLKDLIKSGGEWISSVDLENALMGHPKVKEAAVVAIPHPKWQERPLAVVVPRGEKPTPEELNEHLLKAGFAKWQLPDAYVFAEEIPRTSAGKFLKRALREQYKNYYGGA

SEQ ID NO: 15 - protein, 534 aa, Bradyrhizobium sp.
  source 1..534: note = CI-41S
MDWSQHAIPPMRLEPRFGDRVVPAFVDRPASLWAMIADAVAQNGGGEALVCGDIRISWHEVARRAAKVAAGFAKLGLNSGDRVAILLGNRIEFVLTMFAAAHAGLVTVLLSTRQQKPEIAYVLNDCGARALVHEATLAERIPDAADIPGLAHRIAVSDDAASQFAVLLDHPPAPAPAAVSEEDTAMILYTSGTTGRPKGAMLAHCNIIHSSMVFASTLRLTQADRSIAAVPLAHVTGAVANITTMVRCAGTLIIMPEFKAAEYLKVAARERVSYTVMVPAMYNLCLLQPDFDSYDLSSWRIGGFGGAPMPVATIERLDAKIPGLKLANCYGATETTSPSTLMPGELTAAHIDSVGLPCPGAEIIVMGPDGRELPRGEIGELWIRSASVIKGYWNNPKATAESFTDGFWHSGDLGSVDAENFVRVFDRQKDMINRGGLKIYSAEVESVLAGHPAVIESAIIAKPCPVLGERVHAVIVTRTEVDAESLRAWCAERLSDYKVPETMTLTTTPLPRNANGKVVKRQLRETLAAGQAPA

SEQ ID NO: 16 - protein, 612 aa, Bradyrhizobium sp.
  source 1..612: note = CI-41S
MAGPAVLTVADTIARSFLLAVQTRGDRPAIREKKFGIWQPTSWREWLQISKDIAHGLHASGFRPGDVASIIANAVPEWVYADMGILCAGGVSSGIYPTDSTAQVEYLVNDSRTKIVFVEDEEQLDKVLACRARCPTLEKIVVFDMEGLSGFSDPMVLSFAEFAALGRNHAHGNAALWDEMTGSRTASDLAILVYTSGTTGPPKGAMHSNRSVTHQMRHANDLFPSTDSEERLVFLPLCHVAERVGGYYISIALGSVMNFAESPETVPDNLREVQPTAFLAVPRVWEKFYSGITIALKDATPFQNWMYGRALAIGNRMTECRLEGETPPLSLRLANRAAYWLVFRNIRRMLGLDRCRIALTGAAPISPDLIRWYLALGLDMREVYGQTENCGVATIMPTERIKLGSVGKAAPWGEVMICPKGEILIKGDFLFMGYLNQPERTAETIDAKGWLHTGDVGTIDNEGYVRITDRMKDIIITSGGKNVTPSEIENQLKFSPYVSDAVVIGDKRPYLTCLIMIDQENVEKFAQDHDIPFTNYASLCRAREIQDLIQREVEAVNTKFARVETIKKFYLIERQLTPEDEELTPTMKLKRSFVNKRYAAEIDAMYGARAVA

SEQ ID NO: 17 - protein, 513 aa, Bacillus subtilis
  source 1..513: note = (strain 168)
MNLVSKLEETASEKPDSIACRFKDHMMTYQELNEYIQRFADGLQEAGMEKGDHLALLLGNSPDFIIAFFGALKAGIVVVPINPLYTPTEIGYMLTNGDVKAIVGVSQLLPLYESMHESLPKVELVILCQTGEAEPEAADPEVRMKMTTFAKILRPTSAAKQNQEPVPDDTAVILYTSGTTGKPKGAMLTHQNLYSNANDVAGYLGMDERDNVVCALPMFHVFCLTVCMNAPLMSGATVLIEPQFSPASVFKLVKQQQATIFAGVPTMYNYLFQHENGKKDDFSSIRLCISGGASMPVALLTAFEEKFGVTILEGYGLSEASPVTCFNPFDRGRKPGSIGTSILHVENKVVDPLGRELPAHQVGELIVKGPNVMKGYYKMPMETEHALKDGWLYTGDLARRDEDGYFYIVDRKKDMIIVGGYNVYPREVEEVLYSHPDVKEAVVIGVPDPQSGEAVKGYVVPKRSGVTEEDIMQHCEKHLAKYKRPAAITFLDDIPKNATGKMLRRALRDILPQ

SEQ ID NO: 18 - protein, 622 aa, Azoarcus olearius
METVIRDVGRMFAKPVVNVETRGDGSRILRSGIPLPDTYARCVGEWVEKWGKETPDQLFLAERDAVSGEWRKITWGETRRRVIGIATWLLGQKLSAERPVVILSDNSIEHALLMLAAMHVGVPVSSISPGNSLMSRDHAKLKGNIELLRPGVIFADPVEKFAPALAAIRELHDGVVIAGRNSQPTAGTVPFAEIEVAPDEAAVMAAFNAITPDTIAKFLFTSGSVGVPKAVINTQRMMCSNQLAKELVWPFLKENRPVLVEWLPWSHTFGSNHNLNMILRWGGTIWIDDGKPTPAGLDKTVKNLKEISPTVYFNVPRAYDMLVPLLREDKQLRETFFARLNLIFYAGAALPHHLWEGLEDLSEQTTGHKVTMVSSWGSTETAPMCTDCHFEAERPGVIGVPVPGTALKLVPSADKLEVRVKGPNIFPGYWKQPDITAKSFDEEGYYMIGDAVEFLDERFPEKGLLFDGRVGEDFKLLTGTWVHVGSLRVAGIDAMKPVAQDIVVTGHDRDEIGFLVFPNIPECRTLCPDLPPDADIIDLLLNPAVRQRVRQGMALMKQIGGGSSTYPSRALLMAEPPSVEAGEITDKGYINQRMVLNRRADLVEYLYQDVVDKTVITVHSAI

SEQ ID NO: 19 - protein, 524 aa, Microbacterium oxydans
MVRSTYPDVEIPEVSIHDFLFGDLSEAELDTVALVDGMSGATTTYRQLVGQIDLFAGALAARGVGVGTTVGVLCPNVPAFATVFHGILRAGATATTINSLYTADEIANQLTDAGATWLVTVSPLLPGAQAAAEKLGFDADHVIVLDGAEGHPSLPALLGEGRQAPDVSFDPSTHLAVLPYSSGTTGRPKGVMLTHRNLVANVSQCQPVLGVDASDRVLAVLPFFHIYGMTVLLNFALRQRAGLATMPRFDLPEFLRIIAEHRTSWVFVAPPIAVALAKHPIVDQYDLSAVKVIFSGAAPLDGTLASAVANRLGCIVTQGYGMTETSPAVNLISEARTEIDRSTIGPLVPNTEARLVDPDSGEDVVVPAEGASEPGELWVRGPQVMVGYLNRPDATAEMLDADGWLHTGDVATVTHDGIYRIVDRLKELIKYKGYQVAPAVLEAVLLEHPAIADAAVIGAFDDDGQEVPKAFVVRQPDADLDADAVMAHVTSHVAPHEKVRQVEFIDVIPKSSSGKILRKDLRAR

SEQ ID NO: 20 - protein, 708 aa, Aspergillus niger
  source 1..708: note = (strain CBS 513.88 / FGSC A1513)
MLFSQQPLHLTRADELRQSPPKGTPYSVALPGTEKPGRSKVYRAWNATEGVLKSLDPQILTAHDIFESTANRLPKNHCLGWRPYNPTTKTYGVYQWLDYQTVQKRRAAFGAGLVELHHKHECSRPGQYGIGLWCQNRPEWQITDLACMSQSLYSVSIYDVLAPDATEYIINHAELACVVTSLPHIPTLLRLKPQLPNLKIIVSLDPLDGGEEAGHSKRALLESMAAGQDVSIYTMSQVEELGASVDRPCKPPAPSDTITINYTSGTTGPPKGVVLAHENAVASASGALINSIQKAGDTIISYLPLAHIYARMSEHAAFWAGARIGYFHGNILELVDDLKLLKPTGFISVPRLYTRFGNAIRASTVEAPGFRGALSRHIVATKTANLKNPDPSQATGKHALYDRIWAKKVAAAIGLERSRMLASGSAPLDPSLHQFLRIALGVDVVQGYGLTETYAMACVQSLADLTAGHCGGLIPSTEACLMSLPDMEYSVDDKPYPRGELMLRGANVFREYFKDPEETAKAVTEDGWFRTGDVCKIDEMGRIVIIDRRKNVLKLAQGEYISPERLEGVYMSEMGYLAQGYVHGDSVQTFLVAIFGVQPDTFAVFASKVLGRTIEATDIEGIRSVLNDPKIRKAVLKDLNRIAKKHKLAGYERIKNCALMIDPFTIENNLLTPTLKLKRPPTTKKYRQVLDELYAEALAEESAPKAKL

SEQ ID NO: 21 - protein, 559 aa, Brevibacterium yomogidense
MSWFDERPWLRTLGLTETEAVPLEPSTPLRDLADTVAAHPTTAAWTHYGQSATYAEFDRQTTAFAAYLAESGIRPGDAVAVYAQNSPHFPIATYGIWKAGAVVVPLNPMYRDELTHAFADADVKAIVVQKALYLMRVKEYAADLPLVVLAGDLDWAQDGPDAVFGAYADLPDVPLPDLRTVVDERLDTDFEPLTVRPEDPALIGYTSGTSGKAKGALHPHSSISSNSRMAARNAGLPQGAGVVSLAPLFHITGFICQMIASTANGSTLVLNHRFDPASFLDLLRQEKPAFMAGPATVYTAMMASPSFGADAFDSFHSIMSGGAPLPEGLVKRFEEKTGHYIGQGYGLTETAAQAVTVPHSLRAPVDPESGNLSTGLPQRDAMVRILDDDGNPVGPREVGEVAISGPMVATEYLGNPQATADSLPGGELRTGDVGFMDPDGWVFIVDRKKDMINASGFKVWPREVEDILYMHPAVREGAVVGVPDEYRGETVVAFVSLQPDSQATAEDIIAHCKEHLASYKAPVEVTIVDELPKTSSGKILRRTVRDEATQARQAQPDAH

SEQ ID NO: 22 - protein, 673 aa, Brevibacterium linens
MINNWLAVGLLVVSGILAFNWKRKHPYGQTVEIGEKPENGGRIRRNSACADHLISFLEDDEIYTLYDSLVKSCKKYGERKCFGERKKDSNGNLGKFEWISYNTYLERCEYIQQGLCELGLKPKSKVGIFSKNRLEWLIVHSASFIQSYCVVSFYETLGVESLSYVTEHAEIGLAFCSAETLQKTLDIAKGVKVLKTIICFDSIDKEHYNIAKELGVTLYTYDEIMKKGKEANGKHKHTPPTPDTLSTIMYTSGTTGPPKGVMITHKNLTSVVCAVSDFIKVYDTDVHYSYLPYAHVLERVVILAAFHFGAAIGIFSGDISNILVEVKLLSPTLFIGVPRVFERIKTNVFKEISKKPALLRTLFNGAYNLKYLSIQHGFKLPIIEKVLDLVFFSKIKQALGGKVRVILSGSAPLSFDTEVFLRVVMCCCVLQGYGASEGCGGDACKRLDDESVGTIGPPFASNEIKLVDVPELGYDSNGEVQTGEVCLRGPSISSGYYKDEEKTREEFKDGWFHTGDIGRWNRDGSLSIVDRKKNIFKLSQGEYVAVEKIETIVVKSEYVEQVCIYGDSQKSCVIAIIHPHPESCSEWAGSKKTDKDIKEICKNQDFIKVVLDDIIKNCKKSGLHGFEIPKAIHLTPEAFSDQNNLLTPSFKLKRHEIKKYFEDEIKKLYSKLD

SEQ ID NO: 23 - protein, 555 aa, Nocardioides simplex
  source 1..555: note = (Arthrobacter simplex)
MSFRYYRDLHPTFADRTEWALPTVLRHHAAERPDAVWLDCPEEGRTWTFAETLTAAERVGRSLLAAGAEPGDRVVLVAQNSSAFVRTWLGTAVAGLVEVPVNTAYEHDFLAHQVSTVEATLAVVDDVYAARFVAIAEAAKSIRKFWVIDTGSRDQALATLRDAGWEAAPFEELDEAATAPEVVDATLALPDVRPQDLASVLFTSGTTGPSKGVAMPHAQMYFFADECVSLVRLTPDDAWMSVTPLFHGNAQFMAAYPTLVAGARFVTRSRFSASRWVDQLRESRVTVTNFIGVMMDFIWKQDRRDDDADNPLRVVFAAPTAATLVGPMSERYGIEAFVEVFGLTETSAPIISPYGVDRPAGAAGLAADEWFDVRLVDPETDEEVGVGEIGELVVRPKVPFICSMGYFNMPDKTVEAWRNLWFHTGDALRRDEDGWFYFVDRFKDALRRRGENISSYEIETSILAHPAVVECAVIAVPASSEAGEDEVMAYVITGGDAPVPTPAELWAHCDGRIPSFAVPRYLRFVDEMPKTPSQRVQKAKLRALGVTPDTHDREA

SEQ ID NO: 24 - protein, 515 aa, Pseudomonas putida
  source 1..515: note = (Arthrobacter siderocapsulatus)
MNLGKIITRSARYWPDHTAVADSQTRLTYAQLERRSNRLASGLGALGVATGEHVAILAANRVELVEAEVALYKAAMVKVPINARLSLDEVVRVLEDSCSVALITDATFAQALAERRAALPMLRQVIALEGEGGDLGYAALLERGSEAPCSLDPADDALAVLHYTSGSSGVLKAAMLSFGNRKALVRKSIASPTRRSGPDDVMAHVGPITHASGMQIMPLLAVGACNLLLDRYDDRLLLEAIERERVTRLFLVPAMINRLVNYPDVERFDLSSLKLVMYGAAPMAPALVKKAIELFGPILVQGYGAGETCSLVTVLTEQDHLIEDGNYQRLASCGRCYFETDLRVVNEAFEDVAPGEIGEIVVKGPDIMQGYWRAPALTAEVMRDGYYLTGDLATVDAQGYVFIVDRKKEMIISGGFNVYPSEVEQVIYGFPEVFEAAVVGVPDEQWGEAVRAVVVLKPGAQLDAAELIERCGRALAGFKKPRGVDFVTELPKNPNGKVVRRLVREAYWQHSDRRI

SEQ ID NO: 25 - protein, 390 aa, Dendrobium catenatum
MPSLESIRKAPRANGFASILAIGRANPENFIEQSTYPDFFFRITNSEHLVDLKKKFQRICDKTAIRKRHFVWNEEFITTNPCLHTFMDKSLDVRQEVAIREIPKLGAKAAAKAIQEWGQPKSRITHLIFCTTSGMDLPGADYQLTQILGLNPNVERVMLYQQGCFAGGTTLRLAKCLAESRKGARVLVVCAETTTVLFRGPSEEHQEDLVTQALFADGASALIVGADPDEAAHERASFVIVSTSQVLLPDSAGAIGGHVSEGGLLATLHRDVPKIVSKNVEKCLEEAFTPFGITDWNSIFWVPHPGGRAILDLVEERVGLKPEKLLVSRHVLAEYGNMSSVCVHFALDEMRKRSAIEGKATTGEGLEWGVVFGFGPGLTVETVVLRSVPL

SEQ ID NO: 26 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 27 - protein, 389 aa, Arachis hypogaea
MVSVSGIRKVQRAEGPATVLAIGTANPPNCIDQSTYADYYFRVTNSEHMTDLKKKFQRICERTQIKNRHMYLTEEILKENPNMCAYKAPSLDAREDMMIREVPRVGKEAATKAIKEWGQPMSKITHLIFCTTSGVALPGVDYELIVLLGLDPCVKRYMMYHQGCFAGGTVLRLAKDLAENNKDARVLIVCSENTAVTFRGPSETDMDSLVGQALFADGAAAIIIGSDPVPEVEKPIFELVSTDQKLVPGSHGAIGGLLREVGLTFYLNKSVPDIISQNINDALNKAFDPLGISDYNSIFWIAHPGGRAILDQVEQKVNLKPEKMKATRDVLSNYGNMSSACVFFIMDLMRKRSLEEGLKTTGEGLDWGVLFGFGPGLTIETVVLRSVAI

SEQ ID NO: 28 - protein, 497 aa, Dictyostelium discoideum
  source 1..497: note = AX4
MNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 29 - protein, 394 aa, Spinacia oleracea
MASVDISEIHNVERAKGQANVLAIGTANPPNVMYQADYPDFYFRLTNSEHMTDLKAKFKRICEKTTIKKRYMHISEDILKEKPDLCDYNASSLDIRQVILAKEVPKVGKDAAMKAIEEWGQAMSKITHLIFCTTSGVDIPGADYQLTMLLGLNPSVKRYMLCQQGCHAGGTVLRLAKDLAENNYGSRVLVVCSENTTVCFRGPTETHPDSMVAQALFADGAGAVIVGAYPDESLNERPIFQIVSTAQTILPNSQGAIEGHLRQIGLAIQLLPNVPDLISNNIDKCLVEAFNPIGINDWNSIFWIAHPGGPAILGQVESKLGLQESKLTTTWHVLREFGNMSSACVFFIMDETRKRSLKEGKTTTGDGFDWGVLFGFGPGLTVETVVLRSFPLNQ

SEQ ID NO: 30 - protein, 391 aa, Chenopodium quinoa
MASVQEIRNAQRADGPATILAIGTANPPNEMYQAEYPDFYFRVTESEHMTDLKKKFKRMCERSMIKKRYMHVTEELLKENPHMCDYNASSLNTRQDILATEVPKLGKEAAIKAIKEWGQPRSKITHVIFCTTSGVDMPGADYQLTKLLGLRPSVKRFMLYQQGCYAGGTVLRLAKDIAENNRGARVLVVCAEITVICFRGPTETHLDSMIGQALFGDGAGAVIVGADVDESIERPIFQLVWAAQTILPDSEGAIDGHLREVGLAFHLLKDVPGLISKNIEKALVEAFKPIGIDDWNSIFWVAHPGGPAILDQVESKLELKQDKLRDTRHVLSEFGNMSSACVLFILDEMRNRSLKEGKTTTGEGLDWGVLFGFGPGLTVETVMLHSVPITN

SEQ ID NO: 31 - protein, 392 aa, Cannabis sativa
MASISVDQIRKAQRANGPATVLAIGTANPPTSFYQADYPDFYFRVTKNQHMTELKDKFKRICEKTTIKKRHLYLTEDRLNQHPNLLEYMAPSLNTRQDMLVVEIPKLGKEAAMKAIKEWGQPKSRITHLIFCSTNGVDMPGADYECAKLLGLSSSVKRVMLYQQGCHAGGSVLRIAKDLAENNKGARILTINSEITIGIFHSPDETYFDGMVGQALFGDGASATIVGADPDKEIGERPVFEMVSAAQEFIPNSDGAVDGHLTEAGLVYHIHKDVPGLISKNIEKSLVEALNPIGISDWNSLFWIVHPGGPAILNAVEAKLHLKKEKMADTRHVLSEYGNMSSVSIFFIMDKLRKRSLEEGKSTTGDGFEWGVLFGFGPGLTVETIVLHSLAN

SEQ ID NO: 32 - protein, 396 aa, Plumbago indica
MAPAVQSQSHGGAYRSNGERSKGPATVLAIATAVPPNVYYQDEYADFFFRVTNSEHKTAIKEKFNRVCGTSMIKKRHMYFTEKMLNQNKNMCTWDDKSLNARQDMVIPAVPELGKEAALKAIEEWGKPLSNITHLIFCTTAGNDAPGADFRLTQLLGLNPSVNRYMIYQQGCFAGATALRIAKDLAENNKGARVLIVCCEIFAFAFRGPHEDHMDSLICQLLFGDGAAAVIVGGDPDETENALFELEWANSTIIPQSEEAITLRMREEGLMIGLSKEIPRLLGEQIEDILVEAFTPLGITDWSSLFWIAHPGGKAILEALEKKIGVEGKLWASWHVLKEYGNLTSACVLFAMDEMRKRSIKEGKATTGDGHEYGVLFGVGPGLTVETVVLKSVPLN

SEQ ID NO: 33 - protein, 389 aa, Ziziphus jujuba
MVTVDEIREAQRAKGPATIMAIGTATPPNAIDQSTFTDYYFRITNSDHKTDLKKKFKTICDKSMIKKRYLYLTEEHLKQNPNMSEYMAPSLDVRQEIVIAEVPKLGKEAANKAIKEWGQPKSKITHLVFSTISGVDAPGADYQLTKLLGLNPSVKRIMVYQQGCFAGGTSLRLAKDLAENNKGARVLVVCTEISAINFRGPSETYFDSNVGQILFGDGASAVVVGSDPLVGVEKPLFELVSASQTIIPDSEGNIEGHICEVGLTIRLSKKVPSLISNNIEKSLVEAFNPLGISDWNSIFWIAHPGGPAILDQIELKLGLKPEKLRASRHVLSEYGNMSSATVLFILDEMRKKSIEDGLKTPGEGLEWGVLFGFGPGLTVETVVLHSVTA

SEQ ID NO: 34 - protein, 390 aa, Anoectochilus roxburghii
MPSLESIRKAPRADGLASILAIGRANPDNFMEQSSFPDFFFRITGSDHLVDLKKKFQRICDRTAIRKRHFVWNEEFIKANPCFSTFMDNSLNVRQEVAIREIPKLGAEAATKAIKEWGQPKSRITHLIFCTTSGMDLPGADYQLTRILGLNPNVERVMLYQQGCFAGGTTLRLAKCLAESRKGARVLVVCAETTTVLFRAPSEEHQEDLVTQALFADGASAVIVGADPDEEAHEKASFVIFSTSQVLLPDSEGAIGGHVSEGGLLATLHRDVPQLVSKNVGKCLEEAFTPLGISDWNSIFWVPHPGGRAILDQIEERVGLKPEKLTTSRHVLAEYGNMSSVCVHFVLDEMRKKSSKEGKATTGEGLEWGVLFGFGPGLTVETVVLRSVPL

SEQ ID NO: 35 - protein, 390 aa, Cymbidium hybrid cultivar
MPSLESVKKSNRADGFASILAIGRANPENFIEQSTYPDFFFRVTNSEHLVNLKKKFQRICDKTAIRKRHFVWNEELLNANPCLGTFMDNSLNVRQEFAIREIPKLGAEAATKAIQEWGQPKSRITHLIFCTTSGMDLPGADYQLTQILGLNPNIERVMLYQQGCFAGGTTLRLAKCLAESRKGARVLVVCAETTAVLFRAPSEEHQDDLVTQALFADGASALIVGADPDETAHERASFVIVSTSQVLLPDSAGAIGGHVSEGGLIATLHRDVPQIVSKNVGKCLEEAFTPLGISDWNSIFWVPHPGGRAILDQVEERVGLKPEKLIVSRHVLAEYGNMSSVCVHFALDEMRKRSKKEGKATTGEGLDWGVLFGFGPGLTVETVVLHSVPI

SEQ ID NO: 36 - protein, 390 aa, Phalaenopsis equestris
MPSLDSIKKAPRADGFASILAIGRANPDNIIEQSAYPDFYFRVTNSEHLVDLKKKFQRICEKTAIRKRHFVWNEEFLTSNPCFSTFMDKSLNVRQEVAIREIPKLGAKAATKAIEDWGQPKSRITHLIFCTTSGMDLPGADYQLTQILGLNPNVERVMLYQQGCFAGGTTLRLAKCLAESRKGARVLVVCAETTTVLFRAPSEEHQDDLVTQALFADGASAVIVGADPDEAADERASFVIVSTSQVLLPDSAGAIGGHVSEGGLLATLHRDVPQIVSKNVGKCLEEAFTPFGISDWNSIFWVPHPGGRAILDQVEERVGLKPEKLSVSRHVLAEYGNMSSVCVHFALDEMRKRSANEGKATTGEGLEWGVLFGFGPGLTVETVVLRSVPL

SEQ ID NO: 37 - protein, 390 aa, Dendrobium catenatum
MPSLESIRKAPRANGFASILAIGRANPENFIEQSTYPDFFFRITNSEHLVDLKKKFQRICDKTAIRKRHFVWNEEFITTNPCLHTFMDKSLDVRQEVAIREIPKLGAKAAAKAIQEWGQPKSRITHLIFCTTSGMDLPGADYQLTQILGLNPNVERVMLYQQGCFAGGTTLRLAKCLAESRKGARVLVVCAETTTVLFRGPSEEHQDDLVTQALFADGASALIVGADPDEAAHERASFVIVSTSQVLLPDSAGAIGGHVSEGGLLATLHRDVPKIVSKNVEKCLEEAFTPFGITDWNTIFWVPHPGGRAILDQVEERMGLKPEKLLVSRHVLAEYGNMSSVCVHFALDEMRKRSAIEGKATTGEGLEWGVLFGFGPGLTVETVVLRSVHL

SEQ ID NO: 38 - protein, 390 aa, Oncidium hybrid cultivar
MPSLESTKKAPRSHGFASILAIGRANPENFVEQNAYPDLFFRATNSKHLVNLKKKFQRICDKTAIRKRHFAWNEEFITANPCLQTFMDNSLNVRQEFAITYIPKLGAEAATKAIQEWGQPKSRITHLIFCTTSGMDLPGADYQLTQILGLNPNVERVMLYQQGCFAGGTTLRLAKCLAESRKGARVLVVCAETTAVLFRAPSEEHQDDLVTQALFADGASALIVGADPDEAANERASFIIVSTSQVLLPDSAGAIGGHVSEGGLLATLHRDVPQIVSKNVGKCLEEAFTPLGISDWNSIFWVPHPGGRAILDLVEERVGLKPEKLLVSRHVLAEYGNMSSVCVHFALDEMRRRSAKEGKATTGEGLDWGVLFGFGPGLTVETVVLHSVPI

SEQ ID NO: 39 - protein, 393 aa, Apostasia shenzhenica
MPGVEAVAQNISPARSDGLAAILAIGRANPPNIVEQSSFADLYFRLHNSEHLVDLKKKLQRICDRTAIRKRHFVWDEELLMANPCLRTVTEPSLNARQKVAITEIPKLGAAAATNAIAEWGRPKSDITHLIFCTTSGMDLPGADYQLIRLLGLNDNIQRIMLYQQGCFAGGTVLRLAKVLAESRRSARVLIVCAETTTVLVRSPSVENQDDLVTQALFADGASALIVGADPNAGEKPVFSVFSTSQVLLPDSDGAIGGHVGENGLTATLHRDVPAVISKNVGKCLEEAFTPLGISDWNSIFWAAHPGGRAILDQVEERVGLKPEKMWASRHVLAEYGNMSSVSVHFALDEIRRRSAKEGKATTGDGFEWGVLFGFGPGLTVETVVLRSAPISA

SEQ ID NO: 40 - protein, 394 aa, Paphiopedilum hangianum
MPGLENRKKVEALIRAEGLATIMAIGRANPPNAMEQSTFPDFYFRVTNSEHLVGLKKKFQRICEKTAIRRRHFVWNEEILNANPCLRTHMEPSLNVRQKIAVAEIPKLGAEAASRAIEEWGQPKSRITHLIFCTTSGMDLPGADYKLTRILGLNPNVQRVMLYQQGCFAGGTVLRLAKCFAESRKGARVLVVCSETTTVLVRAPSEDYQDDLVTQALFADGASALIVGADPDEEAKERPIFTIVSTTQVILPDSDGAIGGHLGEGGLTATLHRDVPLIISKNVSKCLEEAFAPLGISDWNSIFWAPHPGGRAILDQVEERVGLKPEKLWASRHVLAEYGNMSSVCVHFVLDEIRKRSAKESKATTGEGFDWGVLFGFGPGLTVETVILRSVPLN

SEQ ID NO: 41 - protein, 393 aa, Apostasia shenzhenica
MPGLQIISKASSRAADGLAAILAIGRANPPNSMDQSSYPEFYFRVMDSDHLVDLKKKFQRICERTAIRKRHFVWNEELLRDNPCLRTFMDSSLNVRQKVAVAEIPKLGAAAAERAIEEWGQPRSGITHLIFCTTSGMDLPGADYQLTKILGLNADVQRVMLYQQGCFAGGTVLRLAKVLAESRKGARVLVVCAETTTVLIRAPSVEHQDDLVTQALFADGASALIVGADPVEEVNERPLFSIISASQVILPDSDGAIGGHLGEGGLTATLHRDVPLIISKNVSKCLEDAFSPLGISDWNSIFWAPHPGGRAILDQVEERVGLKPEKMWASRHVLAEYGNMSSVCVHFVLDEMRKRSAKEGKPTTGEGLEWGVLFGFGPGLTVETVVLRSHPIN

SEQ ID NO: 42 - protein, 387 aa, Phalaenopsis equestris
MPNMESIKKEDGLATIMAIGRALPPNSIDQNSFPDFYFRVHNSEHLMDLKNKFRRICERTAIRKRHFVWNEEVLKQNPCLRTFMEPSLNTRQEIVCSEIPKLGAEAARNAIREWGQPERSITHLIFCTTSGMNLPGADFEAAQILGLNHSVERVMLYQQGCFAGGTVLRLAKCLAESRRGARVLVICAESTTSLVRSPSREHQYDLIAQALFADGASALIIGTEPNAEAGERPIFSIFSTAQVTLPDSGDAIRGYLKEGGLIATLAKDVPLIISENIERCLQEAFGPLGISDWNSIFWAPHPGGRAILDGIEDKLGLKPEKLWAARHVLAEYGNMSSVCVHYILDEMRRRDVKNGKAPTGDGPEWGVLFGFGPGLTVETVVLRRLFL

SEQ ID NO: 43 - protein, 394 aa, Bromheadia finlaysoniana
MASQVSPPSINMAPKADGFASILAIGRANPKNFIEQSTFPDFFFRVTNTEHMVDLKKKFQRICDKTSIRKRHFIWNEELLTANPSLCTFMGNSLNLRHEVAVREIPKLGAEAATKAIQEWGQPKSFITHLVFCTTSGMDLPGADYQLTQILGLNLDIERVMLHQQGCFLGGTTLRLAKYLAESRKGARVLVVCAETTTEFFRAPSEEHQEDLVTQSLFGDGASALIVGADPHEGARERASFILVSSSQVLLANSAHAITGHVSEGGIKATLHRDVPQIISNNLGKCLEEAFTPLGISDWNSIFWVLHPGGRAILDQVEEKMGLEPEKLLISRHVLLEYGNMSSVCVHFALDEMRKRSSNEGKATTGEGLEWGVLFGFGPGLTIETVVLRSVSIS

SEQ ID NO: 44 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 45 - protein, 101 aa, synthetic construct
MAVKHLIVIKFSDSITEAQKEEFFKTYLNLVNIIPAMKDVYWGKDVTRRNKEEGYTHIVEVTFESVETIQDYIIHPAHVGFGDVYRHYWEKLLIFDYTPRK

SEQ ID NO: 46 - protein, 101 aa, synthetic construct
MSVKHLIVIKFSDEITEAQKEELFKTYVNLVNIIPAMKDVYWGKDVRQRNKEEGYTHIVEVTFESVETIQDYIIHPAHVGFGDVYRHYWEKLLIFDYTPRK

SEQ ID NO: 47 - protein, 101 aa, synthetic construct
MSVKHLIVIKFSDEITEAQKEELFKTYVNLVNIIPAMKDVYWGKDVTRRNKEEGYTHIVEVTFESVETIQDYIIHPAHVGFGDVYRHYWEKLLIFDYTPRK

SEQ ID NO: 48 - protein, 101 aa, synthetic construct
MSVKHLIVIKFKDSITEAQKEELFKTYVNLVNIIPAMKDVYWGKDVTRRNKEEGYTHIVEVTFESVETIQDYIIHPAHVGFGDVYRHYWEKLLIFDYTPRK

SEQ ID NO: 49 - protein, 101 aa, synthetic construct
MSVKHLIVIKFKDSITEAQKEELFKTYLNLVNIIPAMKDVYWGKDVTRRNKEEGYTHIVEVTFESVETIQDYIIHPAHVGFGDVYRSYWEKLLIFDYTPRK

SEQ ID NO: 50 - protein, 101 aa, synthetic construct
MAVKHLIVIKFSDSITEAQKEELFKTYVNLVNIIPAMKDVYWGKDVTRRNKEEGYTHIVEVTFESVETIQDYIIHPAHVGFGDVYRHYWEKLLIFDYTPRK

SEQ ID NO: 51 - protein, 101 aa, synthetic construct
MSVKHLIVIKFSDSITEAQKEEFFKTYVNLVNIIPAMKDVYWGKDVTRRNKEEGYTHIVEVTFESVETIQDYIIHPAHVGFGDVYRHYWEKLLIFDYTPRK

SEQ ID NO: 52 - protein, 101 aa, synthetic construct
MSVKHLIVIKFKDSITEAQKEELFKTYVNLVNIIPAMKDVYWGKDVRRRNKEEGYTHIVEVTFESVETIQDYIIHPAHVGFGDVYRSYWEKLLIFDYTPRK

SEQ ID NO: 53 - protein, 404 aa, Cannabis sativa
MSIIIFMGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 54 - protein, 322 aa, Cannabis sativa
MAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 55 - protein, 333 aa, Stachybotrys bisbyi
MPATRTPIHPEAAAYKNPRYQSGPLSVIPKSFVPYCELMRLELPHGNFLGYFPHLVGLLYGSSASPARLPANEVAFQAVLYIGWTFFMRGAGCAWNDVVDQDFDRKTTRCRVRPVARGAVSTTSANIFGFAMVALAFACISPLPAECQRLGLMTTVLSIIYPFCKRVTNFAQVILGMTLAINFILAAYGAGLPAIEAPYTVPTICVTTAITLLVVFYDVVYARQDTADDLKSGVKGMAVLFRNYVEILLTSITLVIAGLIATTGVLVDNGPYFFVFSVAGLLAALLAMIGGIRYRIFHTWNSYSGWFYALAIFNLLGGYLIEYLDQVPMLNKA

SEQ ID NO: 56 - protein, 329 aa, Stachybotrys chartarum
  source 1..329: note = IBT 40288
MSAKVSPMAYTNPRYETGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLVTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 57 - protein, 329 aa, Stachybotrys chlorohalonata
  source 1..329: note = (strain IBT 40285)
MSPKVSSMPYTNPRYESGPLSLIPKSIVPYFELMRFELPHGYYLGYFPHLVGIMYGASAGPERLPARDLVFQALLYVGWTFAMRGAGCAWNDNIDQDFDRKTERCRTRPIARGAVSTTAGHIFAVAGVALAFLCLSPLPTECHQLGVLVTVLSVIYPFCKRFTNFAQVILGMTLAANFILAAYGAGLPALEQPYTRPTMFATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 58 - protein, 329 aa, Stachybotrys chartarum
  source 1..329: note = (strain CBS 109288 / IBT 7711)
MSAKVSPMAYTNPRYERGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLVTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 59 - protein, 321 aa, Cannabis sativa
MAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 60 - protein, 322 aa, Cannabis sativa
MTDTANQTEPPESNTKYSVVTKILSFGHTCWKLQRPYTFIGVISCACGLFGRELFHNTNLLSWSLMLKAFSSLMVILSVNLCTNIINQITDLDIDRINKPDLPLASGEMSIETAWIMSIIVALTGLILTIKLNCGPLFISLYCVSILVGALYSVPPFRWKQNPNTAFSSYFMGLVIVNFTCYYASRAAFGLPFEMSPPFTFILAFVKSMGSALFLCKDVSDIEGDSKHGISTLATRYGAKNITFLCSGIVLLTYVSAILAAIIWPQAFKSNVMLLSHATLAFWLIFQTREFALTNYNPEAGRKFYEFMWKLHYAEYLVYVFI

SEQ ID NO: 61 - protein, 329 aa, Humulus lupulus
MDQRGNSIRASAQIEDRPPESGNLSALTNVKDFVSVCWEYVRPYTAKGVIICSSCLFGRELLENPNLFSWPLIFRALLGMLAILGSCFYTAGINQIFDMDIDRINKPDLPLVSGRISVESAWLLTLSPAIIGFILILKLNSGPLLTSLYCLAILSGTIYSVPPFRWKKNPITAFLCILMIHAGLNFSVYYASRAALGLAFVWSPSFSFITAFITFMTLTLASSKDLSDINGDRKFGVETFATKLGAKNITLLGTGLLLLNYVAAISTAIIWPKAFKSNIMLLSHAILAFSLFFQARELDRTNYTPEACKSFYEFIWILFSAEYVVYLFI

SEQ ID NO: 62 - protein, 324 aa, Humulus lupulus
MPNSLTAWSHQSEFPSTIVTKGSNFGHASWKFVRPIPFVAVSIICTSLFGAELLKNPNLFSWQLMFDAFQGLVVILLYHIYINGLNQIYDLESDRINKPDLPLAAEEMSVKSAWFLTIFSAVASLLLMIKLKCGLFLTCMYCCYLVIGAMYSVPPFRWKMNTFTSTLWNFSEIGIGINFLINYASRATLGLPFQWRPPFTFIIGFVSTLSIILSILKDVPDVEGDKKVGMSTLPVIFGARTIVLVGSGFFLLNYVAAIGVAIMWPQAFKGYIMIPAHAIFASALIFKTWLLDKANYAKEASDSYYHFLWFLMIAEYILYPFIST

SEQ ID NO: 63 - protein, 322 aa, synthetic construct
MAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFCKDVSDIEGDSKHGISTLATRYGAKNITFLCSGIVLLTYVSAILAAIIWPQVFKSNIMILSHAILAFCLIFQTRELALANYASAPSRQFFEFIWLLYYAEYFVYVFI

SEQ ID NO: 64 - protein, 322 aa, synthetic construct
MAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFLKDVPDVEGDKKVGMSTLPVIFGARTIVLVGSGFFLLNYVAAIGVAIMWPQAFKGYIMIPAHAIFASALIFKTRELALANYASAPSRQFFEFIWLLYYAEYFVYVFI

SEQ ID NO: 65 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 66 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDSKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSNWSTATDDELQDIKKHLVFTVRKDIGPFAAPKLIILVDDLPKTRSGKIMRRILRKILAGESDQLGDVSTLSNPGIVRHLIDSVKL

SEQ ID NO: 67 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 68 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 69 - protein, 491 aa, Saccharomyces cerevisiae
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 70 - protein, 527 aa, Saccharomyces cerevisiae
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 71 - protein, 443 aa, Saccharomyces cerevisiae
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 72 - protein, 451 aa, Saccharomyces cerevisiae
MSELRAFSAPGKALLAGGYLVLDPKYEAFVVGLSARMHAVAHPYGSLQESDKFEVRVKSKQFKDGEWLYHISPKTGFIPVSIGGSKNPFIEKVIANVFSYFKPNMDDYCNRNLFVIDIFSDDAYHSQEDSVTEHRGNRRLSFHSHRIEEVPKTGLGSSAGLVTVLTTALASFFVSDLENNVDKYREVIHNLSQVAHCQAQGKIGSGFDVAAAAYGSIRYRRFPPALISNLPDIGSATYGSKLAHLVNEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVIAKQDVDLRAQTADDKRFSKVQWLDVTQADWGVRKEKDPETYLDK

SEQ ID NO: 73 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 74 - protein, 288 aa, Saccharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 75 - protein, 357 aa, Streptomyces aculeolatus
MTTEVTSFTGAGPHPAASVRRITDDLLQRVEDKLASFLTAERDRYAAMDERALAAVDALTDLVTSGGKRVRPTFCITGYLAAGGDAGDPGIVAAAAGLEMLHVSALIHDDILDNSAQRRGKPTIHTLYGDLHDSHGWRGESRRFGEGIGILIGNLALVYSQELVCQAPPAVLAEWHRLCSEVNIGQCLDVCAAAEFSADPELSRLVALIKSGRYTIHRPLVMGANAASRPDLAAAYVEYGEAVGEAFQLRDDLLDAFGDSTETGKPTGLDFTQHKMTLLLGWAMQRDTHIRTLMTEPGHTPEEVRRRLEDTEVPKDVERHIADLVEQGRAAIADAPIDPQWRQELADMAVRAAYRTN